(19) United States
(12) Patent Application Publication
(10) Pub. No.: US 2025/0303095 A1
(43) Pub. Date: Oct. 2, 2025

(54) ASSEMBLY APPARATUS

(71) Applicant: ResMed Asia Pte. Ltd., Singapore (SG)

(72) Inventors: Han Cheng LIN, Singapore (SG); Sipu CHEN, Singapore (SG); Robin YEW, Singapore (SG); Alex Jun Chen CHIA, Singapore (SG)

(21) Appl. No.: 19/091,505

(22) Filed: Mar. 26, 2025

(30) Foreign Application Priority Data
Mar. 28, 2024 (AU) 2024900838
May 3, 2024 (AU) 2024901287
Nov. 21, 2024 (AU) 2024903830

(51) Int. Cl. A61M 16/06

(52) U.S. Cl. CPC A61M 16/0616; A61M 16/0688; A61M 2202/0007; A61M 2202/02; A61M 2205/0216; A61M 2207/00; A61M 2209/088

(57) ABSTRACT

Forms of the present technology relate to an assembly apparatus for applying an adhesive layer to a patient interface for use in delivering breathable gas to a patient. The assembly apparatus, which may be portable, may comprise a first assembly component comprising a first receiving region configured to receive at least a portion of the patient interface, and a second assembly component comprising a second receiving region configured to receive at least a portion of the adhesive layer. The first assembly component may be configured to engage with the second assembly component to position the first receiving region adjacent to the second receiving region to apply the adhesive layer to a seal-forming structure of the patient interface to form an assembled patient interface. The adhesive layer may be configured to adhere the assembled patient interface to the patient's face.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use. The present technology relates to seal-forming structures for patient interfaces which form a seal with a patient's airways through adhesive surfaces. The present technology also relates to patient interfaces with mechanisms for promoting closure of the mouth during use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

A range of therapies have been used to treat or ameliorate such conditions. Furthermore, otherwise healthy individuals may take advantage of such therapies to prevent respiratory disorders from arising. However, these have a number of shortcomings.

One of the major issues in respiratory therapy is adherence, which is also referred to as compliance. Usually, a patient may be required to don a patient interface for prolonged periods as part of the respiratory therapy. Bulky and/or obtrusive patient interfaces often lead to patients discontinuing the respiratory therapy due to discomfort, inconvenience or interference with sleep. In particular, it is difficult to ensure that infants and children do not remove patient interface during respiratory therapy.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and/or data management.

### 1.2.3.1 Patient Interface

A patient interface may be used to interface respiratory equipment to its wearer, for example by providing a flow of air to an entrance to the airways. The flow of air may be provided to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure.

Conventionally, mask systems are used as patient interfaces to convey the flow of air. These mask systems typically include a plenum chamber which is secured against the patient's face through headgear. The plenum chamber, with the patient's face, encloses a volume of space, which may accommodate the facial features of the patient such as their nose and/or mouth. Often, the plenum chamber may be made of a rigid material. These aspects of the design of some conventional patient interfaces can make sleeping while wearing the patient interface on inconvenient, uncomfortable and potentially claustrophobic for the patient.

Mask systems other than those typically used for respiratory therapy may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. As mentioned earlier, this discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy.

It is often recommended that a patient regularly wash their mask, if a mask is required to be cleaned, or if it is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and/or a nasal bridge region of a face. These different types of patient interfaces may be known by a variety of names by their manufacturer including nasal cushions, nasal pillows, and nasal puffs.

In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These patient interfaces may be referred in the art as oral cushions, oro-nasal cushions or full face cushions.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a molded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. A seal formed by an adhesive is usually highly effective with little or no leak for typical therapy pressures (e.g. up to 20 cmH₂O).

The adhesive on a seal-forming structure may lose its adhesiveness with repeated use and over time. Therefore, there may be a need to refresh the adhesive on the seal-forming structure. Due to its adhesive nature, refreshing the adhesive may be a fiddly job that is difficult to perform effectively and doing so may create residue on a user's hands. The adhesive may also tend to cause the seal-forming structure to undesirably stick to itself, another part of the patient interface or another object. It may be difficult to unstick the seal-forming structure and, after becoming unstuck, the effectiveness of the adhesive may be reduced.

A range of patient interface seal-forming structure technologies are disclosed in the following patent applications, assigned to ResMed Pty Ltd: WO 1998/004,310; WO 2006/074,513; WO 2010/135,785. Examples of patient interfaces including seal-forming structures which use an adhesive to achieve a seal are disclosed in PCT Publication No. WO 2023/015340, the contents of which are herein incorporated by reference.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. Examples of patient interfaces which use an adhesive to position and stabilise a seal-forming structure with the face are disclosed in PCT Publication No. WO 2023/015340, the contents of which are herein incorporated by reference. One advantage of the use of adhesives to position and stabilise the seal-forming structure on the patient's face is that it avoids the need for headgear (discussed below), which can be uncomfortable, claustrophobic and adds manufacturing cost and complexity. However, as mentioned before, the use of adhesives, as is known in the art, has some disadvantages.

Another technique is the use of one or more straps and/or stabilising harnesses. Many such harnesses suffer from being one or more of ill-fitting, bulky, uncomfortable and awkward to use. They tend to be less air-tight than adhesive-based seal forming structures. Moreover, straps and/or stabilising harnesses tend to leave markings on the face when used overnight.

### 1.2.3.1.3 Pressurised Air Conduit

In one type of treatment system, a flow of pressurised air is provided to a patient interface through a conduit in an air circuit that fluidly connects to the patient interface so that, when the patient interface is positioned on the patient's face during use, the conduit extends out of the patient interface forwards away from the patient's face. This may sometimes be referred to as a “tube down” configuration.

Conduits connecting to an interface at the front of a patient's face may sometimes be vulnerable to becoming tangled up in bed clothes.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus, RPT devices may also act as flow therapy devices. Examples of RPT devices include a CPAP device and a ventilator.

1.2.3.3 Air circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

An aspect of certain forms of the present technology is to provide methods and/or apparatus that improve the compliance of patients with respiratory therapy.

An aspect of one form of the present technology is a patient interface comprising a seal-forming structure which is configured to form a seal with a region of the patient's face surrounding an entrance to the patient's airways.

Another aspect of one form of the present technology is a patient interface comprising a seal-forming structure having an opening such that a flow of breathable gas is delivered to at least an entrance to the patient's nares.

Another aspect of one form of the present technology is a patient interface comprising a seal-forming structure which further comprises at least one adhesive surface configured in use to adhere to the region of the patient's face surrounding the entrance to the patient's airways to form the seal.

An aspect of one form of the present technology is a patient interface comprising a plenum chamber pressurisable to a therapeutic pressure of at least 6 cmH₂O above ambient air pressure, said plenum chamber including a plenum chamber inlet port configured to receive a flow of breathable gas at the therapeutic pressure for breathing by the patient.

In one form of the present technology, the seal-forming structure is configured to maintain said therapeutic pressure in the plenum chamber throughout the patient's respiratory cycle in use.

Another aspect of one form of the present technology is a patient interface that has a perimeter shape which is complementary to that of an intended wearer. In one form, the seal-forming structure is configured to have a perimeter shape which is complementary to the region of the patient's face surrounding the entrance to the patient's airways to form the seal. The regions to which the seal-forming structure is to be adhered to may be referred to as the target sealing regions. In one form, the seal-forming structure is configured such that the region of the patient's face comprises regions of the patient's face adjacent to, or surrounding, the nares.

According to one aspect of the technology there is provided a patient interface for use in delivering breathable gas to a patient. The patient interface may comprise a plenum chamber pressurisable to a therapeutic pressure of at least 6 cmH₂O above ambient air pressure. The plenum chamber may include a plenum chamber inlet port configured to receive a flow of breathable gas at the therapeutic pressure for breathing by the patient. The patient interface may further comprise a seal-forming structure provided to the plenum chamber. The seal-forming structure may be configured to form a seal with a region of the patient's face surrounding an entrance to the patient's nares. The seal-forming structure may have an opening therein such that the flow of breathable gas is delivered to the entrance to the patient's nares. The seal-forming structure may be configured to maintain said therapeutic pressure in the plenum chamber throughout the patient's respiratory cycle in use. The seal-forming structure may comprise at least one adhesive surface configured in use to adhere to a region of the patient's face to form the seal. The patient interface may further comprise a vent structure to allow a continuous flow of gases exhaled by the patient from an interior of the plenum chamber to ambient. The vent structure may be configured to maintain the therapeutic pressure in the plenum chamber in use.

In certain forms, the seal-forming structure may comprise at least one adhesive surface configured in use to adhere to a region of the patient's face to form the seal.

According to one aspect of the technology there is provided a patient interface for use in delivering breathable gas to a patient. The patient interface may comprise a plenum chamber pressurisable to a therapeutic pressure of at least 6 cmH₂O above ambient air pressure. The plenum chamber may include a plenum chamber inlet port configured to receive a flow of breathable gas at the therapeutic pressure for breathing by the patient. The patient interface may further comprise a seal-forming structure. The seal-forming structure may be configured to form a seal with a region of the patient's face surrounding an entrance to the patient's airways. The seal-forming structure may have an opening therein such that the flow of breathable gas is delivered to at least an entrance to the patient's nares. The seal-forming structure may be configured to maintain said therapeutic pressure in the plenum chamber throughout the patient's respiratory cycle in use. The patient interface may further comprise a vent structure to allow a continuous flow of gases exhaled by the patient from an interior of the plenum chamber to ambient. The vent structure may be configured to maintain the therapeutic pressure in the plenum chamber in use. The seal-forming structure may comprise at least one patient-facing adhesive surface configured in use to adhere to a region of the patient's face to form the seal. The seal-forming structure may further comprise at least one non-patient-facing adhesive surface formed around the opening and configured to adhere to a patient-facing side of the plenum chamber. The seal-forming structure may further comprise a non-patient-facing removable layer positioned over the non-patient-facing adhesive surface and configured to be removed prior to adhering the plenum chamber to the seal-forming structure.

In certain forms, the seal-forming structure may further comprise a patient-facing removable layer positioned over the patient-facing adhesive surface and configured to be removed prior to adhering the seal-forming structure to the patient's face.

In certain forms, the non-patient-facing removable layer may be formed with a hole therein. The hole may substantially align with the opening when the non-patient-facing removable layer is positioned over the non-patient-facing adhesive surface.

In certain forms, the non-patient-facing removable layer may comprise a tab configured to be grasped by a user (e.g. a patient) for removing the non-patient-facing removable layer from the seal-forming structure.

An aspect of certain forms of the present technology is an assembly apparatus for assembling a patient interface for use in delivering breathable gas to a patient. The assembly apparatus may be configured to bring two portions of the patient interface into engaging contact. The two portions may be engaged together by means of adhesive. In some forms, the assembly apparatus may be portable. In other forms, the assembly apparatus may be non-portable.

According to one aspect of the technology there is provided a portable assembly apparatus for assembling a patient interface for use in delivering breathable gas to a patient. The portable assembly apparatus may comprise a first assembly component which may comprise a first receiving region which may be configured in use to receive a first portion of the patient interface. The portable assembly apparatus may further comprise a second assembly component which may comprise a second receiving region which may be configured in use to receive a second portion of the patient interface. The first assembly component may be configured to engage with the second assembly component to position the first receiving region adjacent to the second receiving region in a position suitable to adhere the first and second portions together to form an assembled patient interface.

In certain forms, the first receiving region may comprise a substantially continuous surface against which a substantial part of the first portion of the patient interface may substantially abut when the first portion is received by the first receiving region.

In certain forms, the first receiving region may have a shape that may substantially correspond to a natural shape of a non-patient-facing surface of a seal-forming structure of the patient interface to substantially maintain the shape of the seal-forming structure when forming the assembled patient interface.

In certain forms, the first receiving region may be substantially convex when viewed in cross-section from a side direction.

In certain forms, the first receiving region may comprise one or more concave regions.

In certain forms, the second receiving region may comprise a substantially continuous surface against which a substantial part of a patient-facing surface of a seal-forming structure of the patient interface may substantially abut when the first and second portions are adhered together.

In certain forms, the second receiving region may have a shape that may be substantially complementary to a shape of the first receiving region such that the first and second receiving regions may be capable of being placed in a meshed configuration.

In certain forms, the second receiving region may have a shape that may substantially correspond to a natural shape of a patient-facing surface of a seal-forming structure of the patient interface which may substantially maintain the shape of the seal-forming structure when forming the assembled patient interface.

In certain forms, the second receiving region may be substantially concave when viewed in cross-section from a side direction.

In certain forms, the second receiving region may comprise one or more convex regions.

In certain forms, the first receiving region and/or the second receiving region may be formed, at least in part, from a resiliently deformable material.

In certain forms, the first assembly component may comprise a first retaining structure which may be configured to maintain the first portion in a substantially fixed position with respect to the first assembly component.

In certain forms, the first retaining structure may comprise a recess which may be configured to substantially receive the first portion.

In certain forms, the first retaining structure may comprise a cavity which may be configured to receive a plenum chamber of the patient interface.

In certain forms, the second assembly component may comprise a second retaining structure which may be configured to maintain the second portion in a substantially fixed position with respect to the second assembly component.

In certain forms, the second retaining structure may comprise a first slot and/or a second slot. The first slot may be configured to receive a first tab of the second portion or a removable layer connected thereto in use. The second slot may be configured to receive a second tab of the second portion or a removable layer connected thereto in use.

In certain forms, the first assembly component and the second assembly component may be movable relative to each other such that the portable assembly apparatus may have a stable configuration. In the stable configuration, the first portion, when received by the first receiving region, and the second portion, when received by the second receiving region, may face each other with a gap separating them.

In certain forms, the first assembly component may comprise an outer member and an inner member which may be configured to move relative to the outer member to bring the first receiving region into a position adjacent to the second receiving region to adhere the first and second portions together. The inner member may comprise the first receiving region.

In certain forms, the portable assembly apparatus may further comprise an elastic member which may be configured to return the outer member and the inner member to an original configuration when a force causing the inner member to move relative to the outer member is removed. The original configuration may be the stable configuration.

In certain forms, the first assembly component may be hingedly attached to the second assembly component.

In certain forms, the first assembly component may be detached from the second assembly component.

In certain forms, the second assembly component may comprise a first assembly part comprising the second receiving region and a second assembly part. In a configuration in which the first assembly part and the second assembly part are connected together, the first assembly part and the second assembly part together may form a cavity suitable for storing one or more patient interface portions.

According to one aspect of the technology there is provided a method for assembling a patient interface for use in delivering breathable gas to a patient. The method may comprise a step of providing a first portion of the patient interface to a first receiving region of a first assembly component of a portable assembly apparatus. The method may further comprise a step of providing a second portion of the patient interface to a second receiving region of a second assembly component of the portable assembly apparatus. The method may further comprise a step of engaging the first assembly component with the second assembly component to position the first receiving region adjacent to the second receiving region in a position suitable to adhere the first and second portions together to form an assembled patient interface.

In certain forms, the step of engaging the first assembly component with the second assembly component may further comprise a sub-step of placing the first receiving region and the second receiving region in a meshed configuration.

In certain forms, the method may further comprise a step of providing the first portion to a first retaining structure of the first assembly component to maintain the first portion in a substantially fixed position with respect to the first assembly component.

In certain forms, the step of providing the first portion to the first retaining structure may further comprise a sub-step of providing the first portion substantially within a recess of the first retaining structure.

In certain forms, the step of providing the first portion to the first retaining structure may further comprise providing a plenum chamber of the patient interface to a cavity of the first retaining structure.

In certain forms, the method may further comprise a step of providing the second portion to a second retaining structure of the second assembly component to maintain the second portion in a substantially fixed position with respect to the second assembly component.

In certain forms, the step of providing the second portion to the second retaining structure may comprise inserting a first tab of the second portion, or a removable layer connected thereon, into a first slot of the second retaining structure. In certain forms, the step of providing the second portion to the second retaining structure may further comprise a inserting a second tab of the second portion, or a removable layer connected thereto, into a second slot of the second retaining structure.

In certain forms, prior to adhering the first and second portions together, the method may further comprise a step of moving the first assembly component and the second assembly component into a stable configuration. In the stable configuration, the first portion, when received by the first receiving region, and the second portion, when received by second receiving region, may face each other with a gap separating them.

In certain forms, the step of moving the first assembly component and the second assembly component into the stable configuration may comprise rotating the first assembly component relative to the second assembly component.

In certain forms, after the step of moving the first assembly component and the second assembly component into the stable configuration, the method may further comprise a step of moving an inner member of the first assembly component relative to an outer member of the first assembly component to bring the first assembly component into engagement with the second assembly component. The inner member may comprise the first receiving region.

In certain forms, after adhering the first and second portions together, the method may comprise a step of removing a removable layer from the second portion to expose an adhesive surface of the second portion for adhering the assembled patient interface to the patient's face.

An aspect of certain forms of the present technology is a medical device that is easy to use, e.g. by a person who does not have medical training, by a person who has limited dexterity, vision or by a person with limited experience in using this type of medical device.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

FIG. 1 shows a system including a patient 1000 wearing a patient interface 3000, receiving a supply of air at positive pressure from an RPT device 4000. The patient is sleeping lying on their side.

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideo trapezius.

FIG. 2L shows an anterolateral view of a nose.

FIG. 3 shows a patient interface which is configured to adhere to an alar rim region of a patient's face in accordance with one form of the present technology.

FIG. 4A shows a perspective view of a patient interface which is adhered to a patient's face in accordance with one form of the present technology.

FIG. 4B shows a side view of the patient interface of FIG. 4A.

FIG. 5 a perspective view of a patient interface which is adhered to an alar crease region of a patient's face in accordance with one form of the present technology.

FIG. 6 is a front view illustration of a patient interface according to another form of the technology.

FIG. 7 is a perspective view illustration of the patient interface shown in

FIG. 20 with another component.

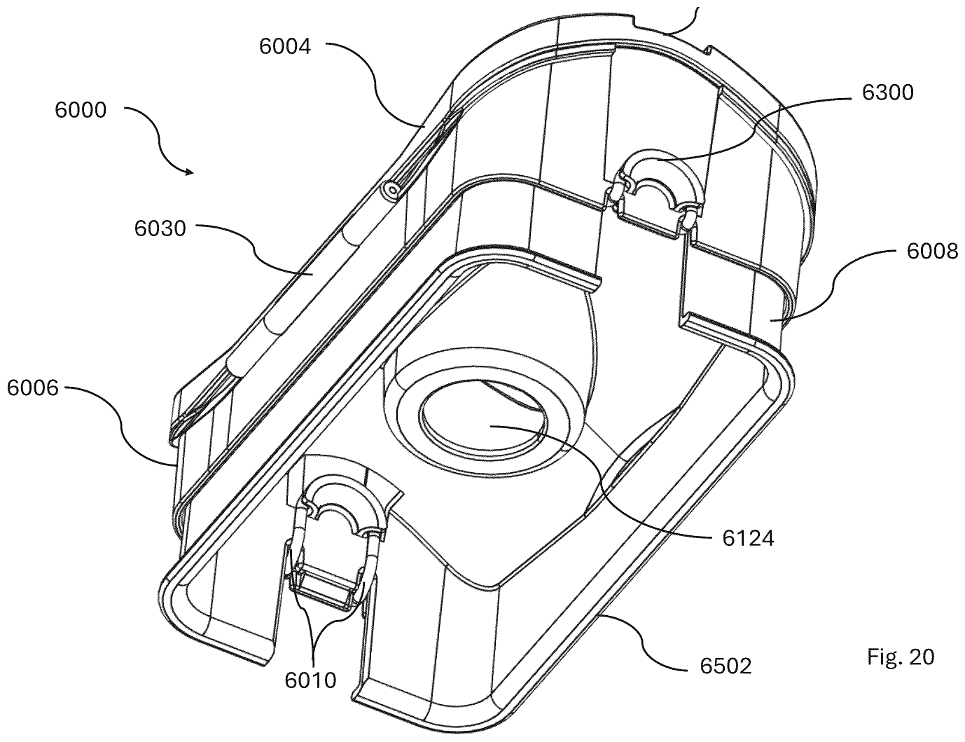

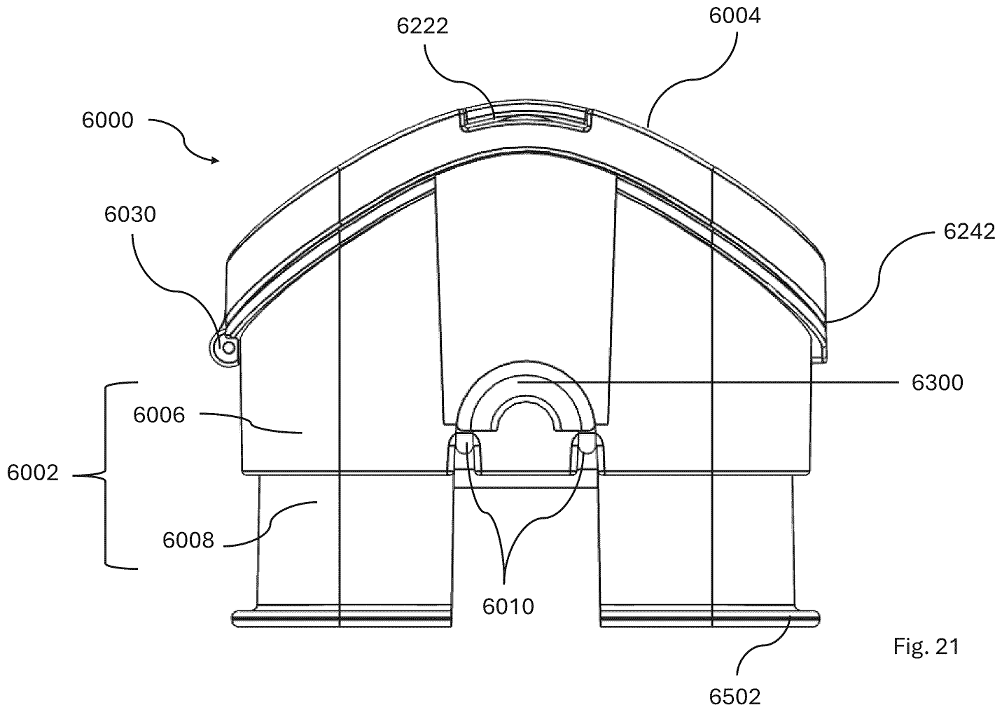

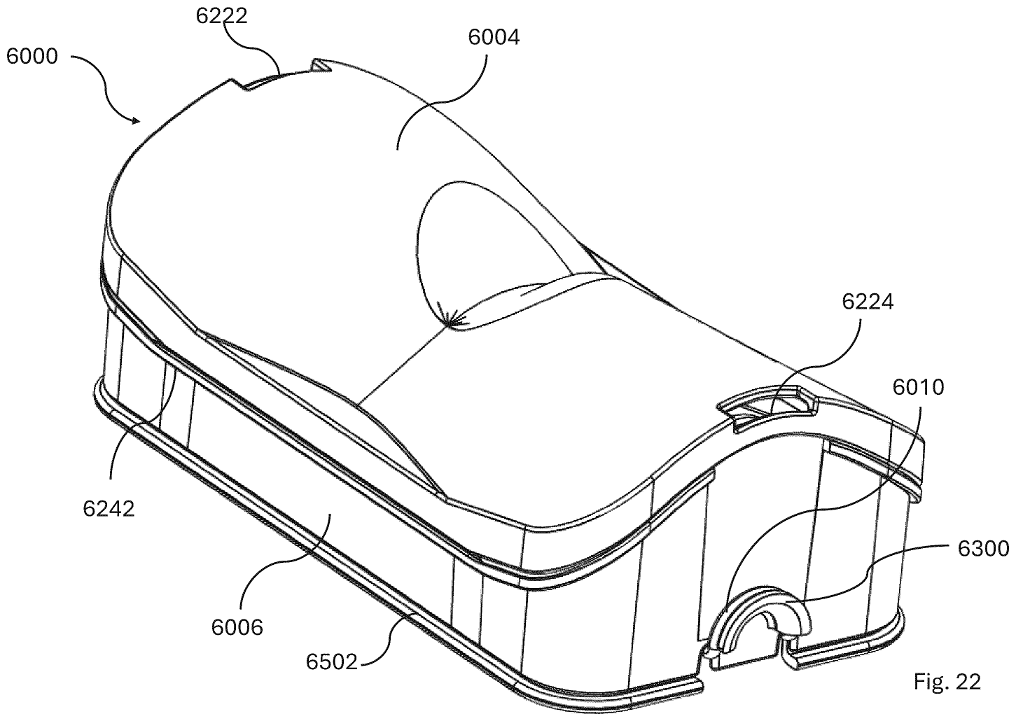

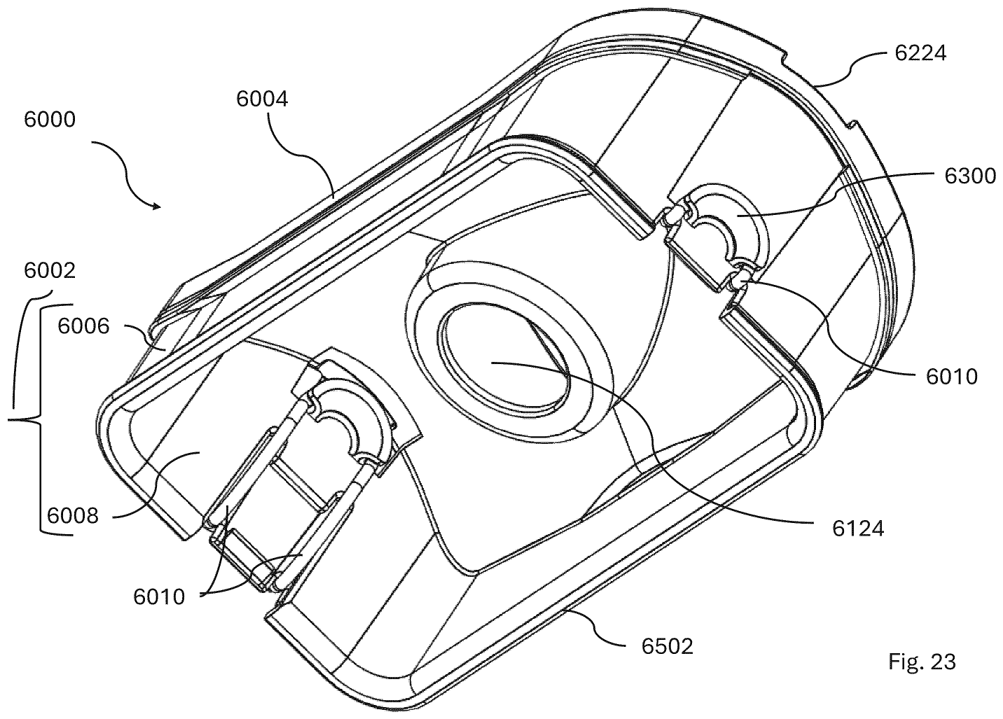

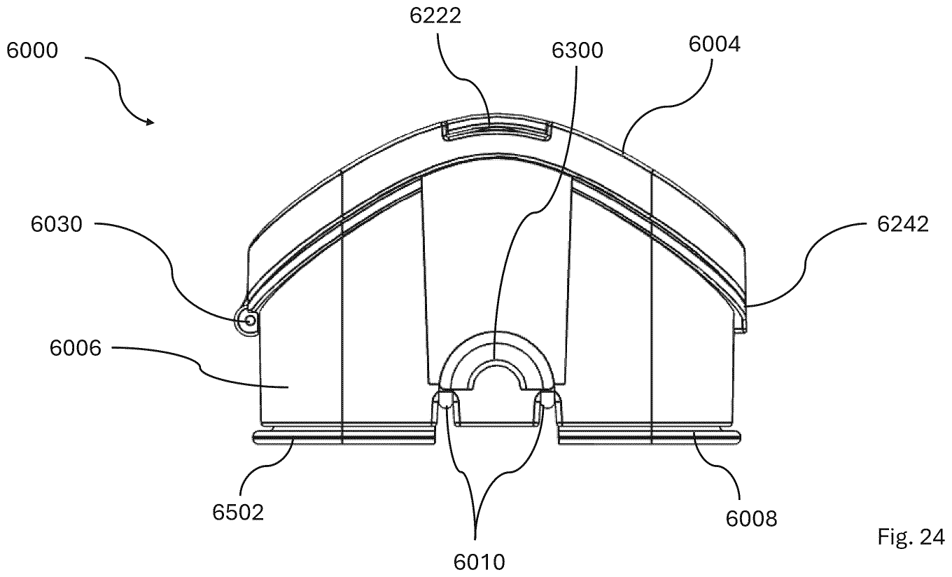

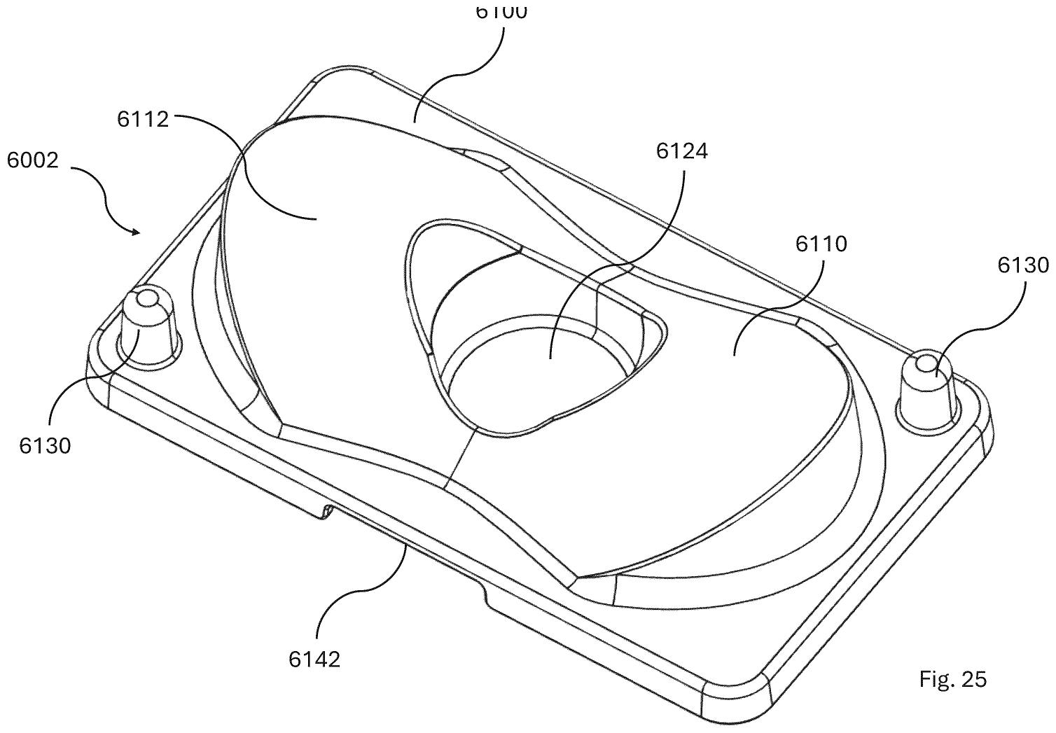

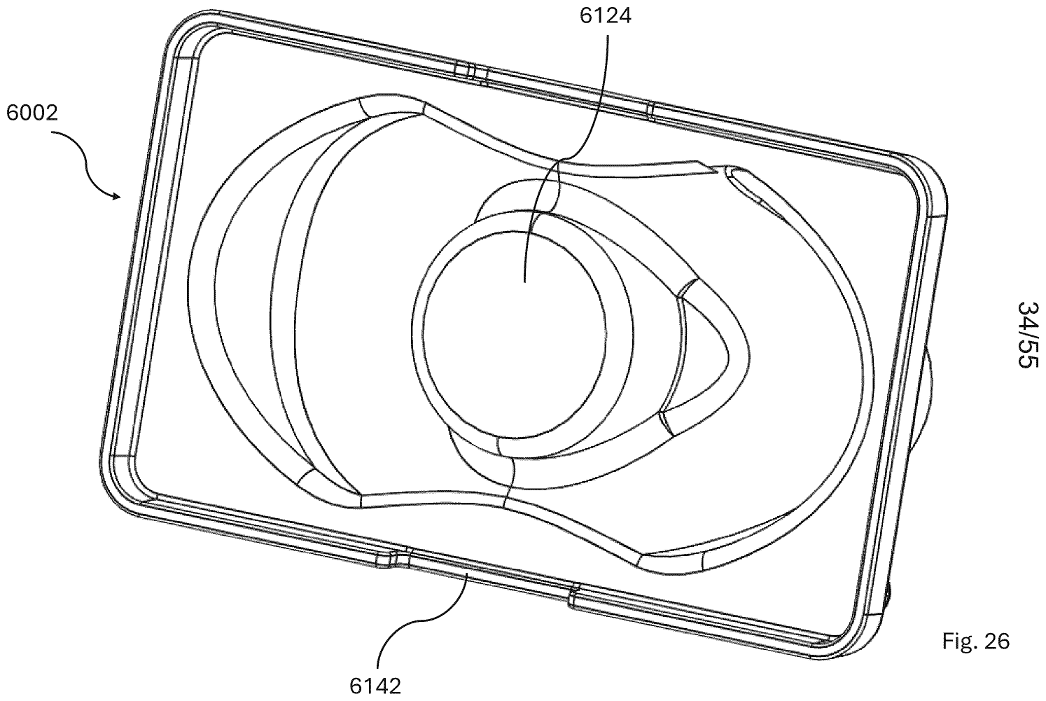

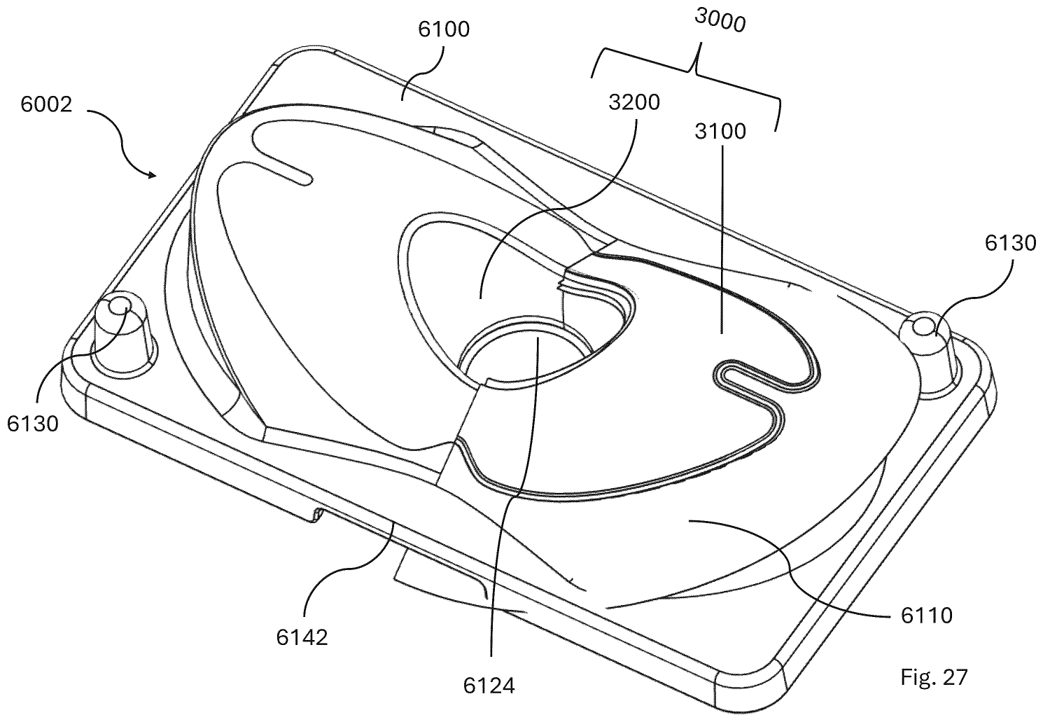

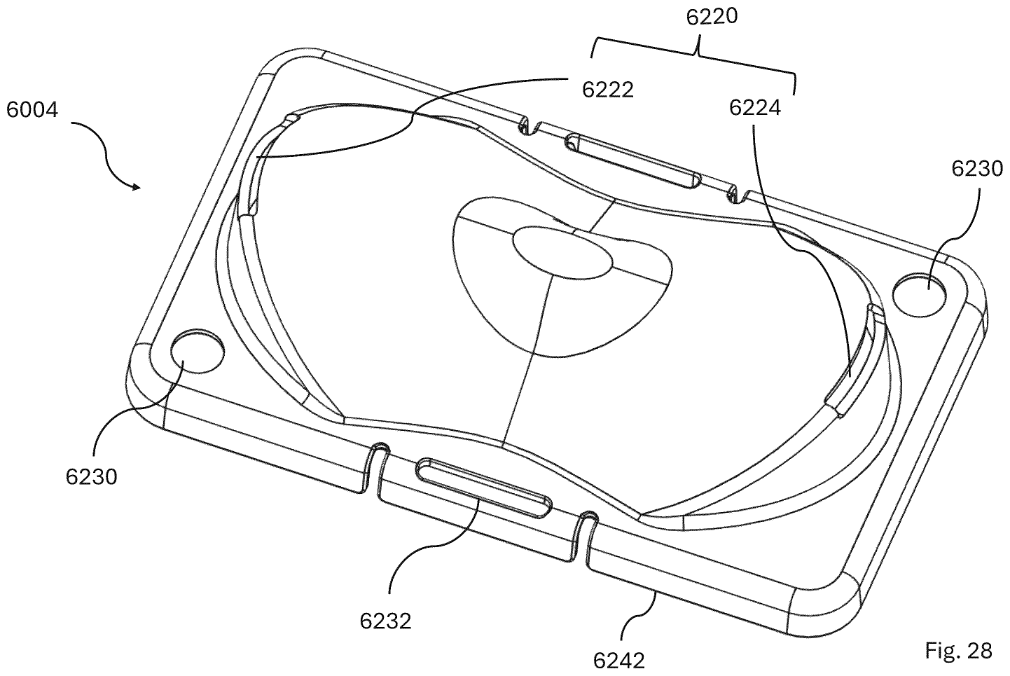

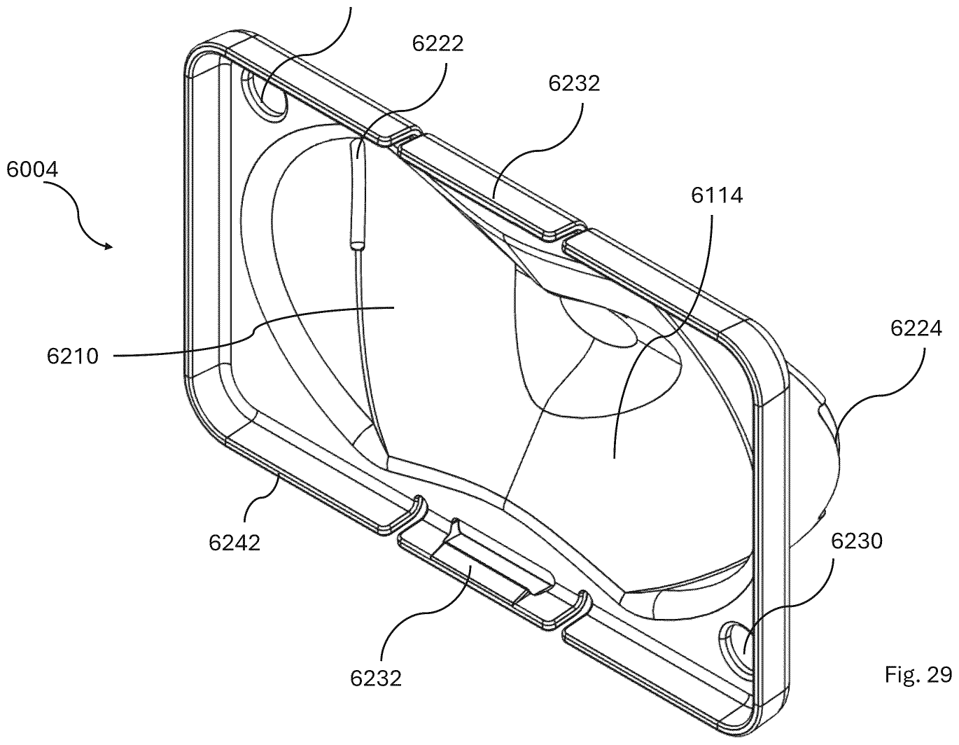

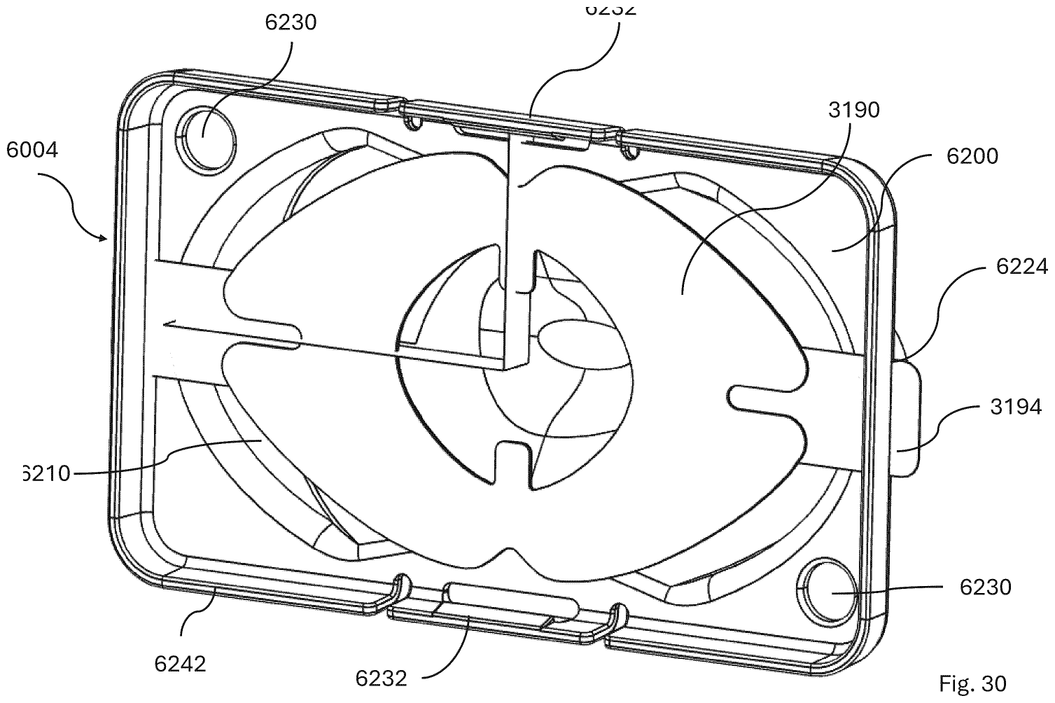

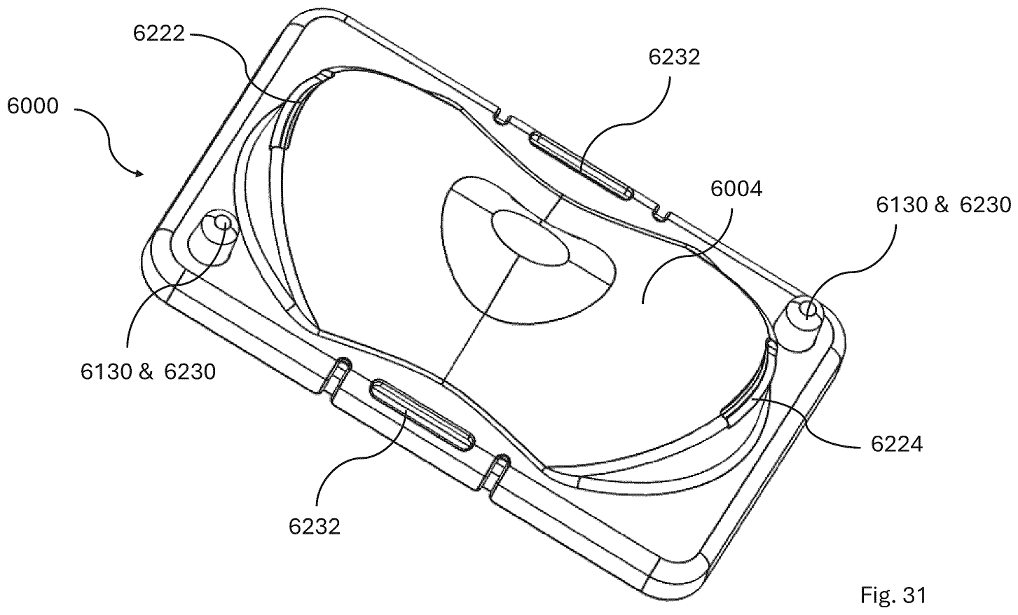

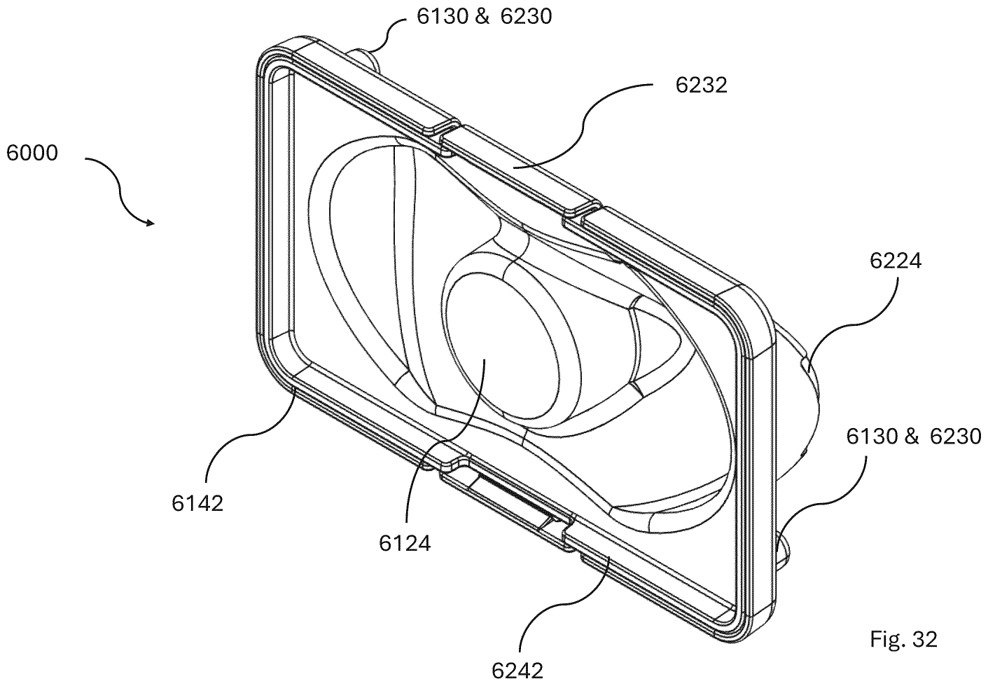

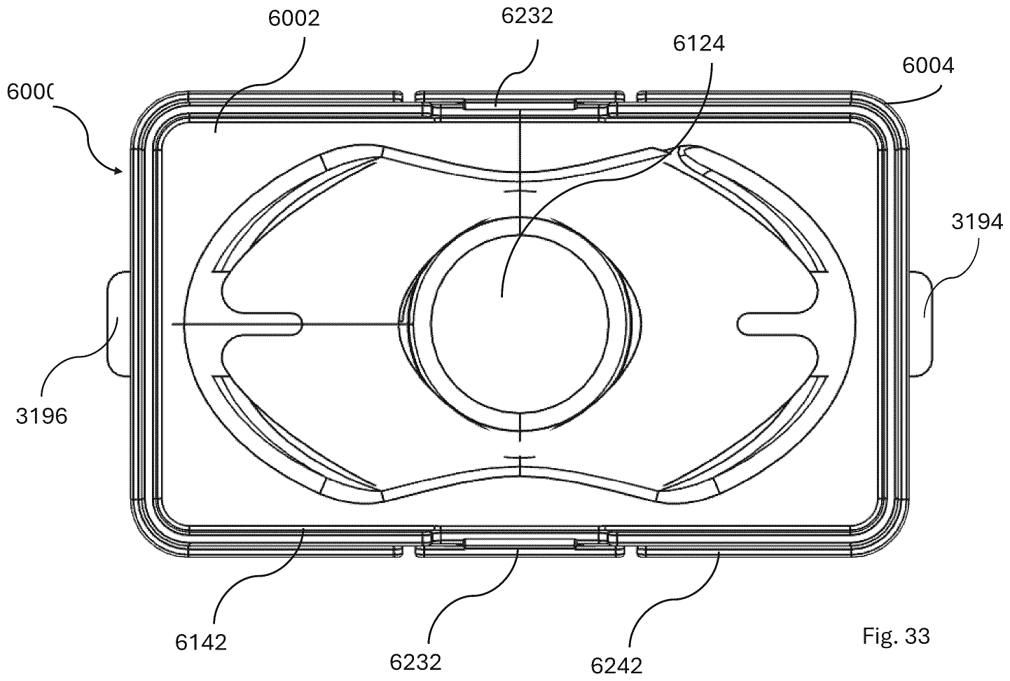

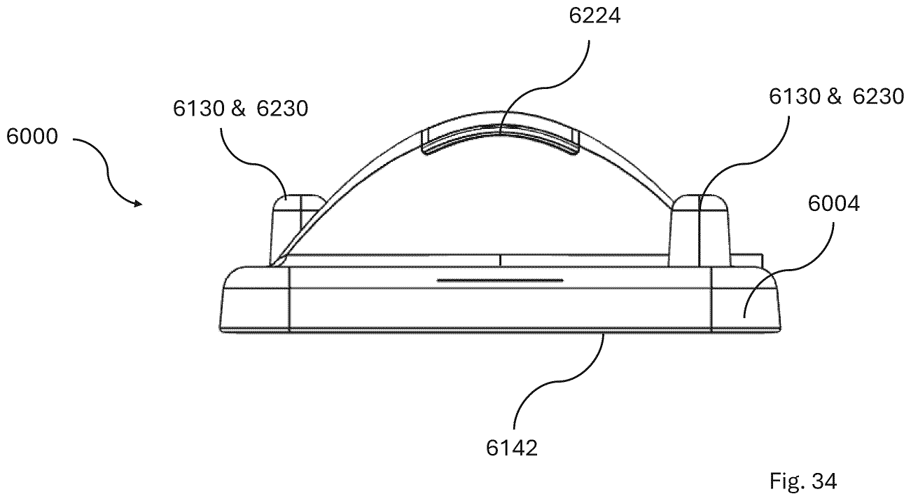

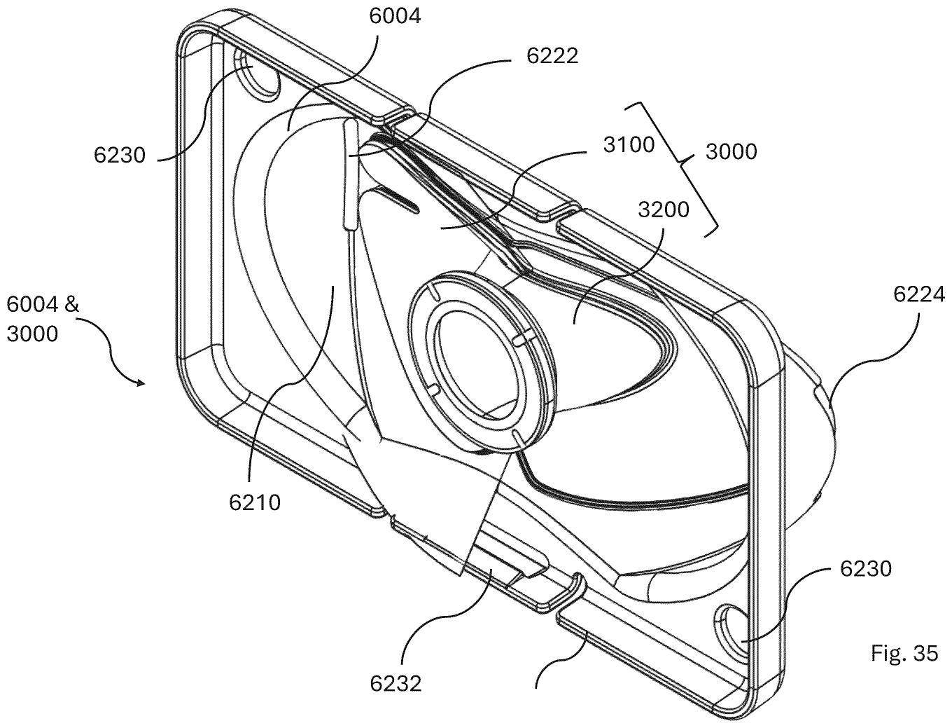

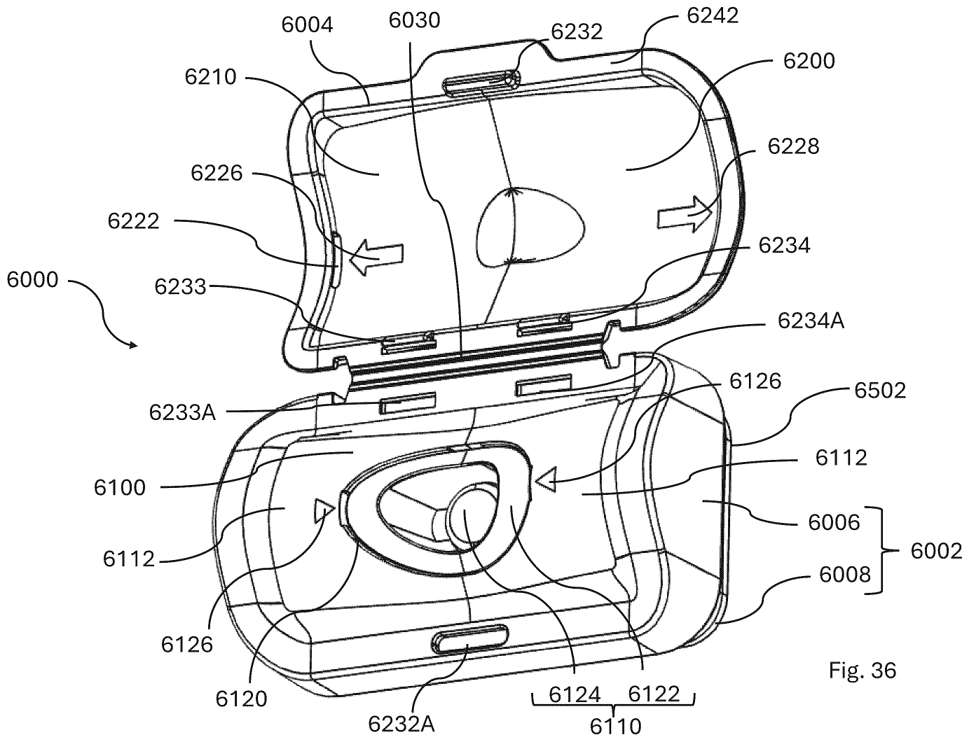

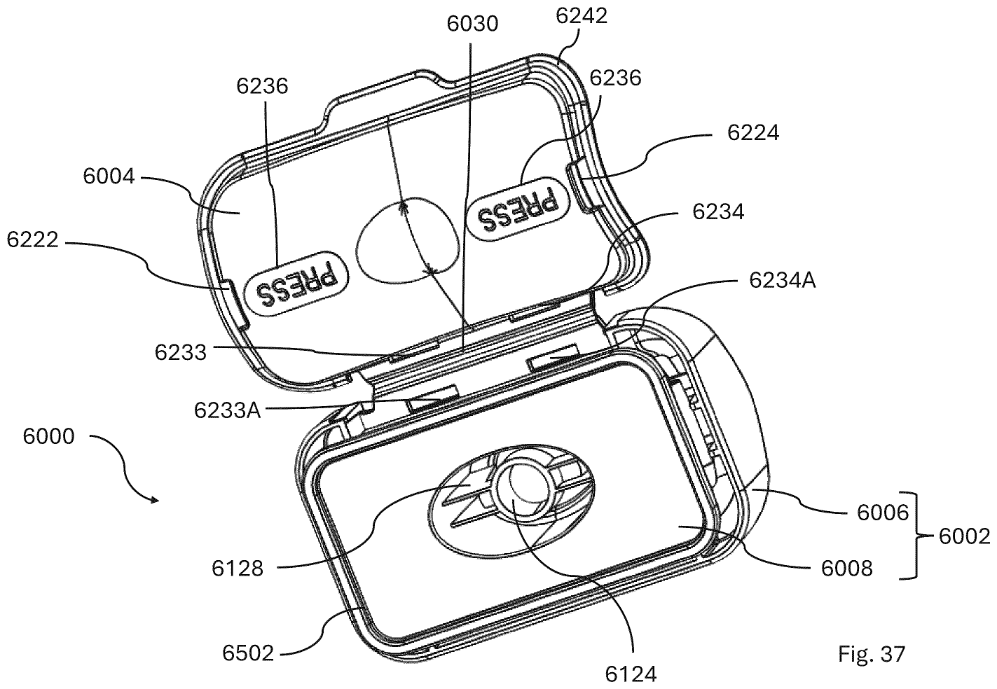

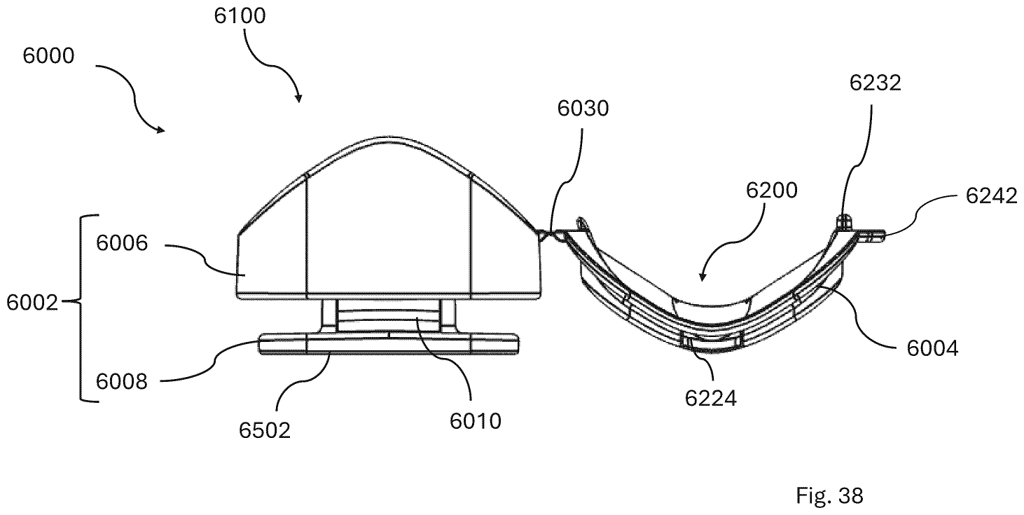

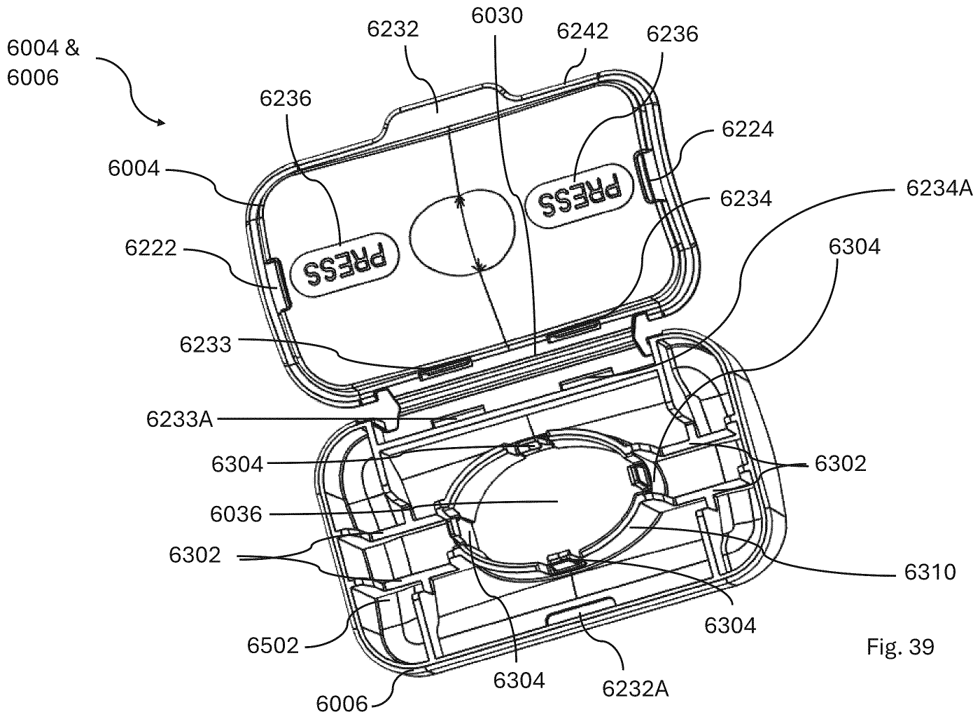

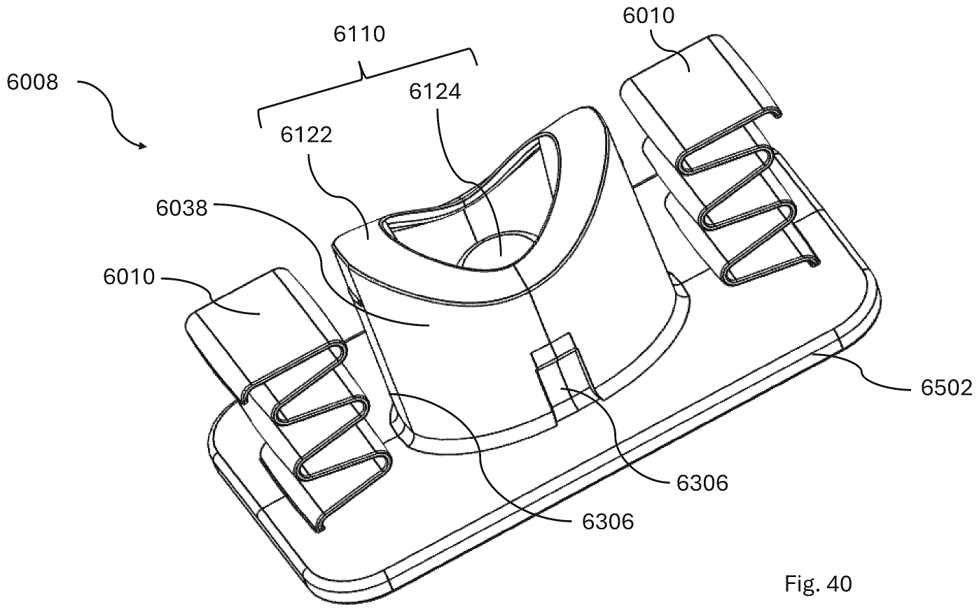

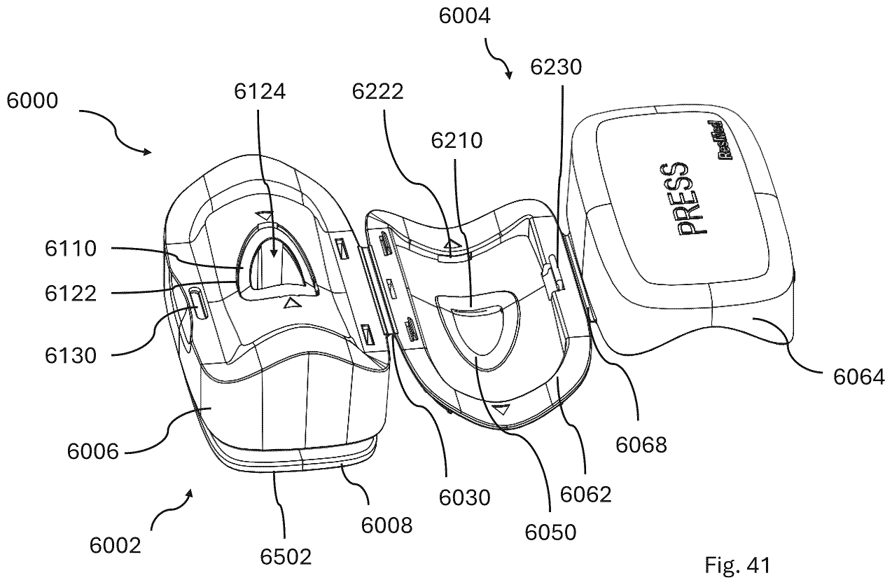

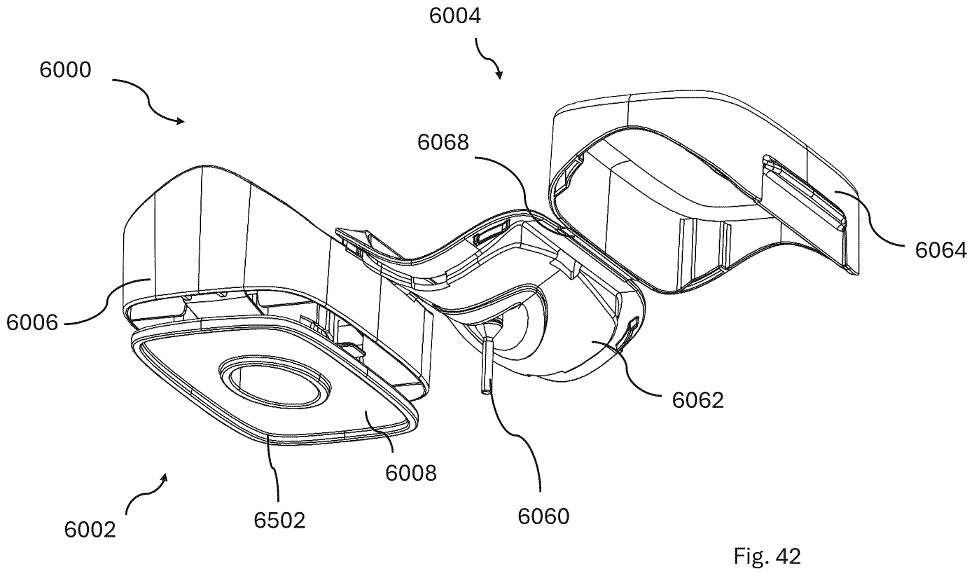

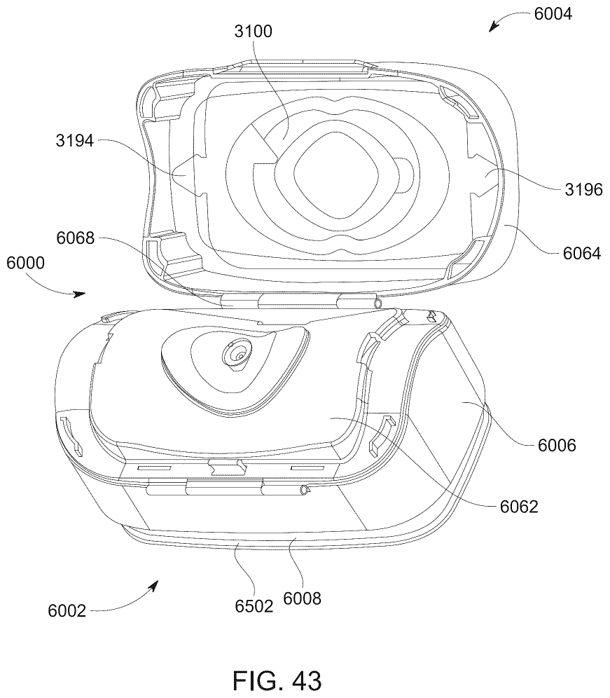

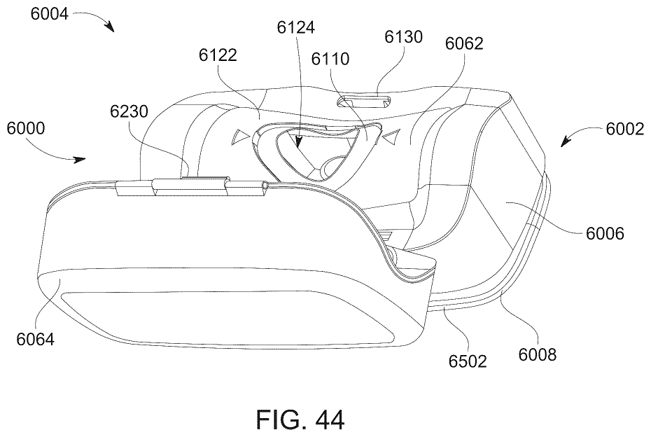

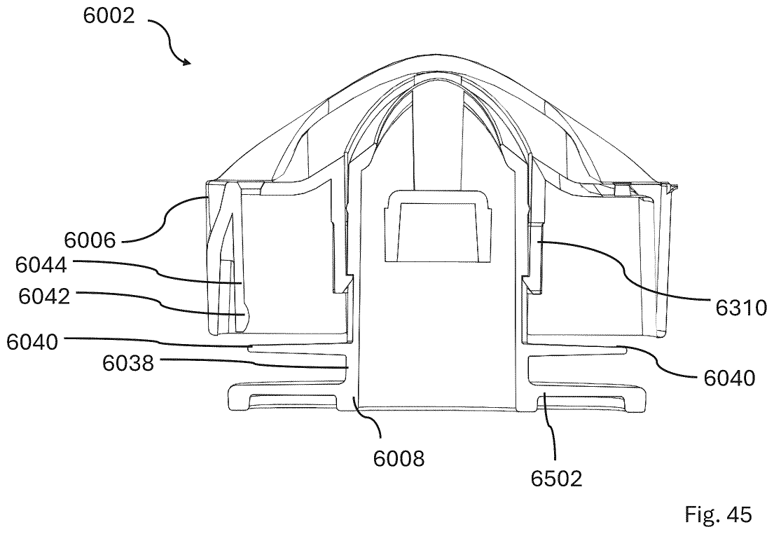

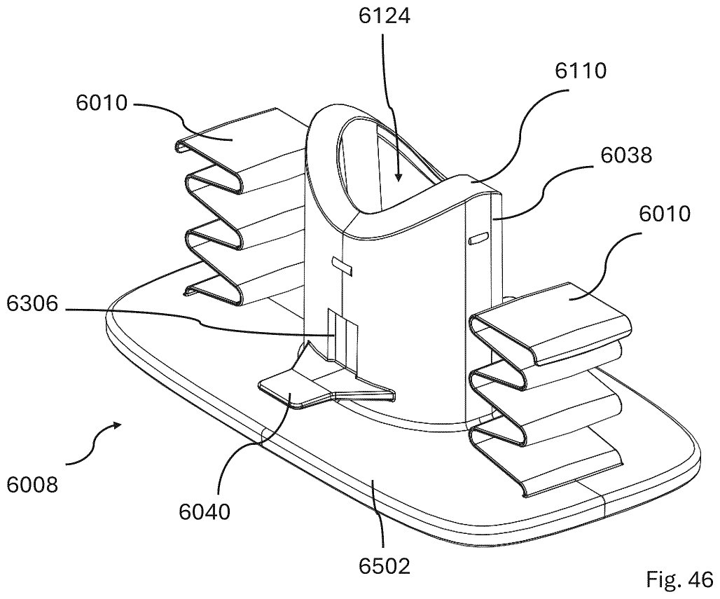

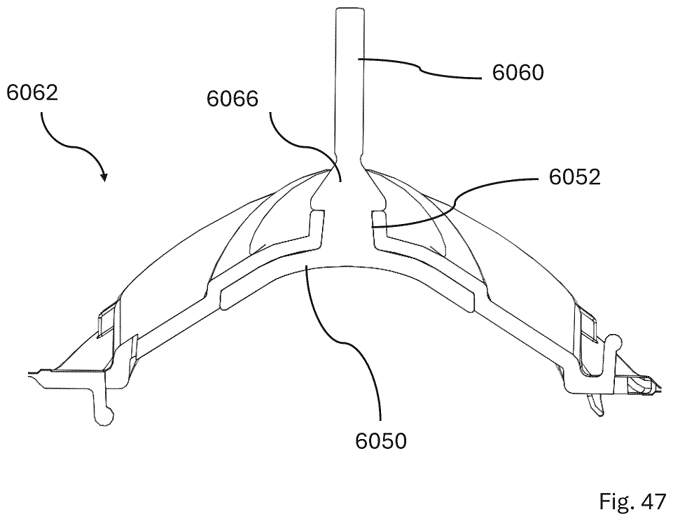

FIG. 8 is an exploded view illustration of the patient interface shown in FIG. 20.

FIG. 9 is an exploded view illustration of a patient interface prior to assembly according to another form of the technology.

FIG. 10 is an exploded view illustration of a patient interface according to another form of the technology.

FIG. 11 is a perspective view illustration of the patient interface shown in FIG. 23 when worn by a patient.

FIG. 12A shows part of the patient interface shown in FIG. 10 when assembled (top part of figure) and in an exploded view (bottom part of figure).

FIG. 12B shows a perspective view of an adhesive layer according to a form of the technology.

FIG. 13 shows a perspective view of a second assembly component of an assembly apparatus according to a form of the technology.

FIG. 14 shows a perspective view of an inner member of a first assembly component of the assembly apparatus according to a form of the technology.

FIG. 15 shows a perspective view of an outer member of the first assembly component of the assembly apparatus according to a form of the technology.

FIG. 16 shows a top perspective view of the assembly apparatus in an open configuration.

FIG. 17 shows a bottom perspective view of the assembly apparatus in the open configuration of FIG. 16.

FIG. 18 shows a side view of the assembly apparatus in the open configuration of FIG. 16.

FIG. 19 shows a top perspective view of the assembly apparatus in a

closed configuration.

FIG. 20 shows a bottom perspective view of the assembly apparatus in the closed configuration of FIG. 19.

FIG. 21 shows a side view of the assembly apparatus in the closed configuration of FIG. 19.

FIG. 22 shows a top perspective view of the assembly apparatus in an assembly configuration.

FIG. 23 shows a bottom perspective view of the assembly apparatus in the assembly configuration of FIG. 22.

FIG. 24 shows a side view of the assembly apparatus in the assembly configuration of FIG. 22.

FIG. 25 shows a top perspective view of a first assembly component of an assembly apparatus according to another form of the technology.

FIG. 26 shows a bottom perspective view of the first assembly component of FIG. 25.

FIG. 27 shows a top perspective view of the first assembly component of FIG. 25 assembled with a patient interface.

FIG. 28 shows a top perspective view of a second assembly component of the assembly apparatus according to another form of the technology.

FIG. 29 shows a bottom perspective view of the second assembly component of FIG. 28.

FIG. 30 shows a bottom perspective view of the second assembly component of FIG. 28 assembled with an adhesive layer.

FIG. 31 shows a top perspective view of the assembly apparatus in a meshed configuration.

FIG. 32 shows a bottom perspective view of the assembly apparatus in the meshed configuration of FIG. 31.

FIG. 33 shows a bottom view of the assembly apparatus in the meshed configuration of FIG. 31

FIG. 34 shows a side view of the assembly apparatus in the meshed configuration of FIG. 31.

FIG. 35 shows a bottom perspective of an assembled patient interface which is removably attached to the second assembly component of FIG. 28.

FIG. 36 shows a top perspective view of an assembly apparatus according to another form of the technology.

FIG. 37 shows a bottom perspective view of the assembly apparatus of FIG. 36.

FIG. 38 shows a side view of the assembly apparatus of FIG. 36.

FIG. 39 shows a bottom perspective view of a second assembly component and an outer member of the first assembly component of the assembly apparatus of FIG. 36.

FIG. 40 shows a top perspective view of an inner member of the first assembly component of the assembly apparatus of FIG. 36.

FIG. 41 shows a top perspective view of an assembly apparatus according to another form of the technology in an open configuration.

FIG. 42 shows a bottom perspective view of the assembly apparatus of FIG. 41.

FIG. 43 shows a top perspective view of an assembly apparatus according to another form of the technology in an open configuration.

FIG. 44 shows a top perspective view of the assembly apparatus of FIG. 43 in another open configuration.

FIG. 45 shows a cross-sectional view of part of an assembly apparatus according to another form of the technology.

FIG. 46 shows a top perspective view of part of the assembly apparatus shown in FIG. 45.

FIG. 47 shows a cross-sectional view of part of an assembly apparatus according to another form of the technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a

respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

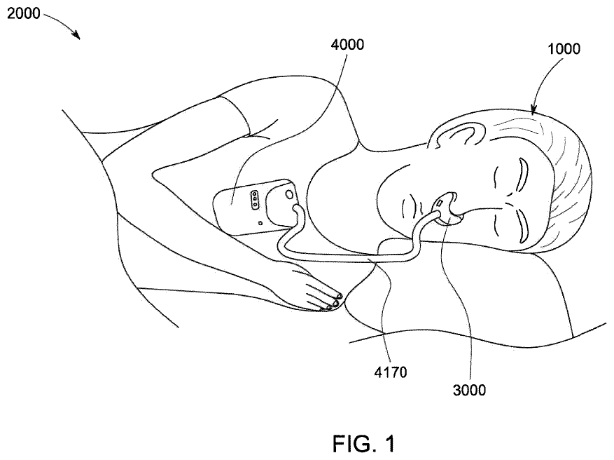

In certain forms, as shown in FIG. 1, the present technology comprises a respiratory therapy system 2000 for treating a respiratory disorder. The respiratory therapy system 2000 may comprise an RPT device 4000 for supplying a flow of air to the patient 1000 via an air circuit 4170 and a patient interface 3000.

In the form of the technology shown in FIG. 1, the RPT device 4000 is portable and can be carried by the patient 1000, for example attached to the patient's clothing. In alternative forms of the present technology (not shown in the Figs.), the RPT device is configured to rest on a nearby surface during use, for example a bedside table.

Further, the respiratory therapy system 2000 may include a humidifier to change the absolute humidity of air or gas for delivery to a patient relative to ambient air. Typically, the humidifier is used to increase the absolute humidity and increase the temperature of the flow of air (relative to ambient air) before delivery to the patient's airways.

### 4.3 Patient Interface

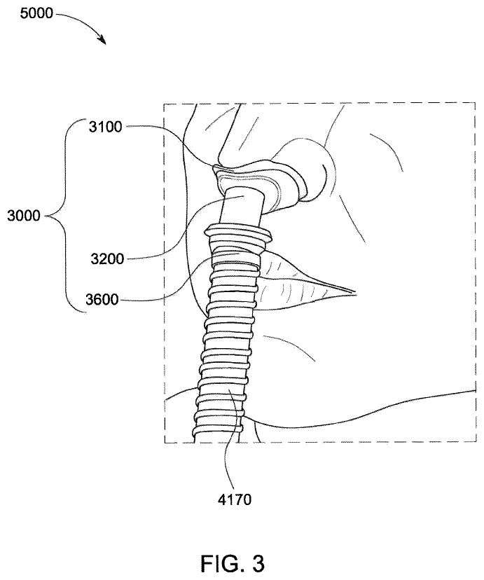

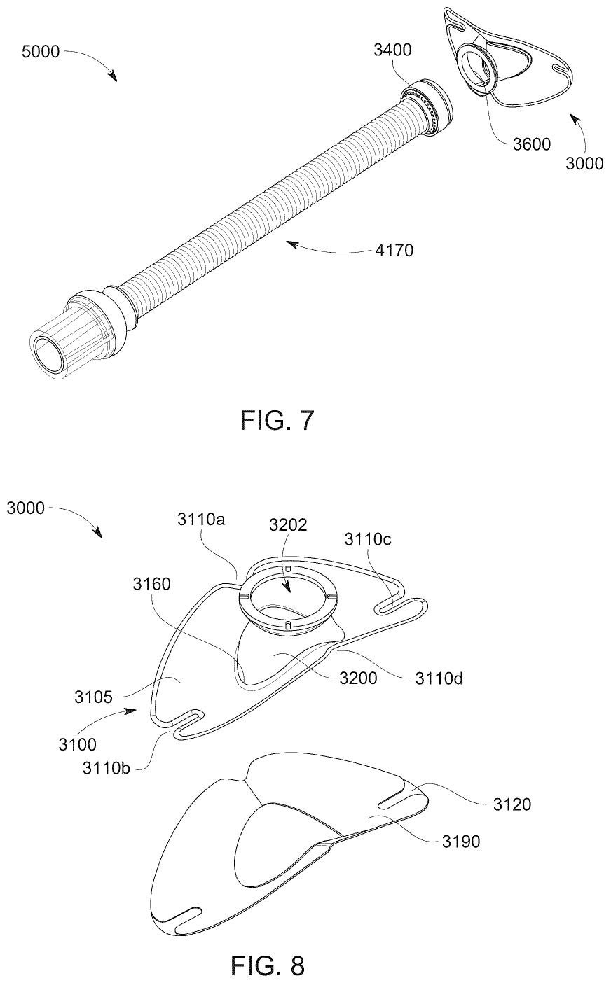

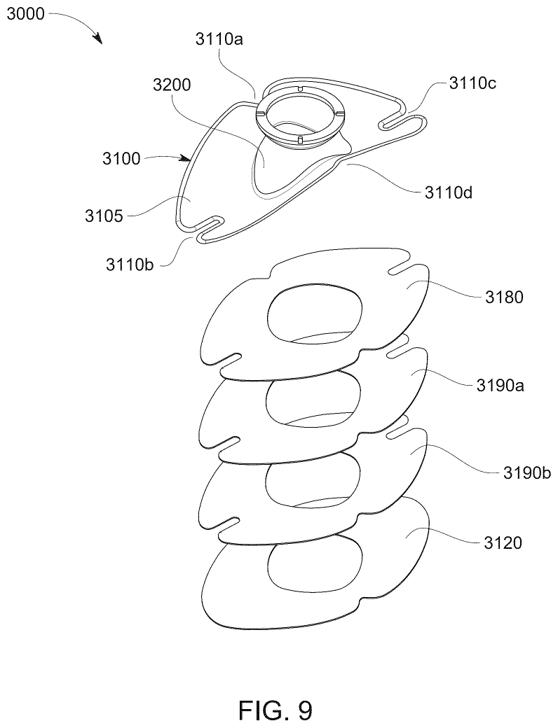

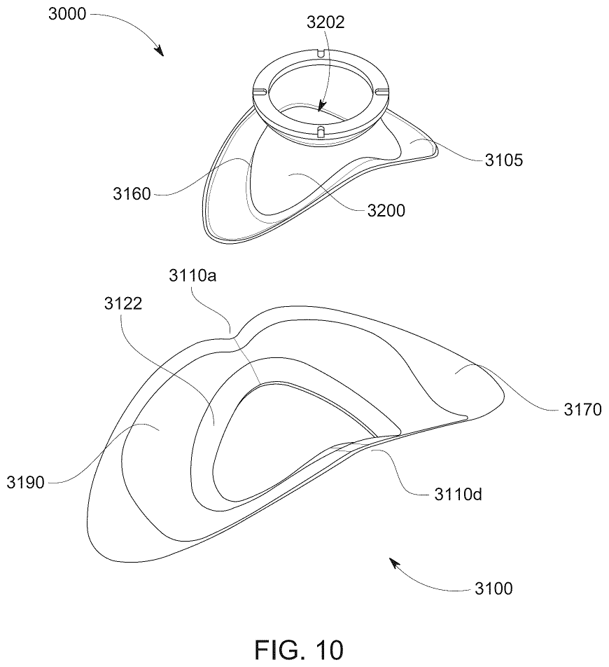

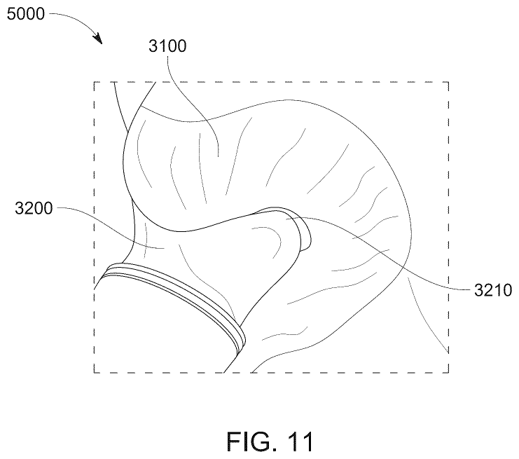

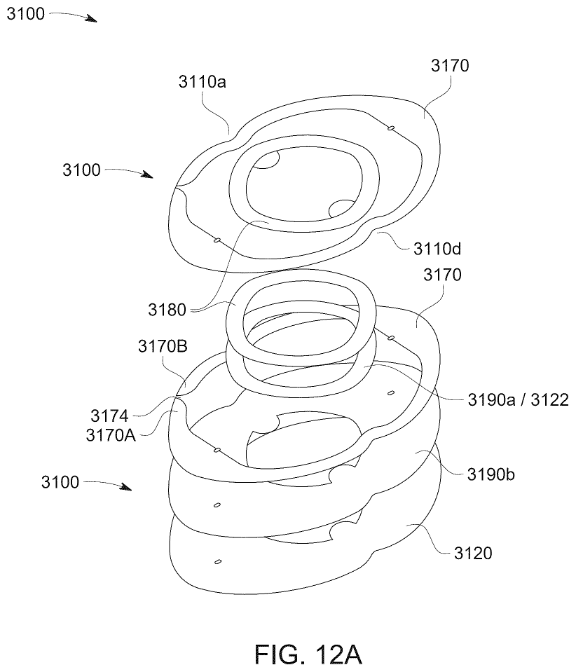

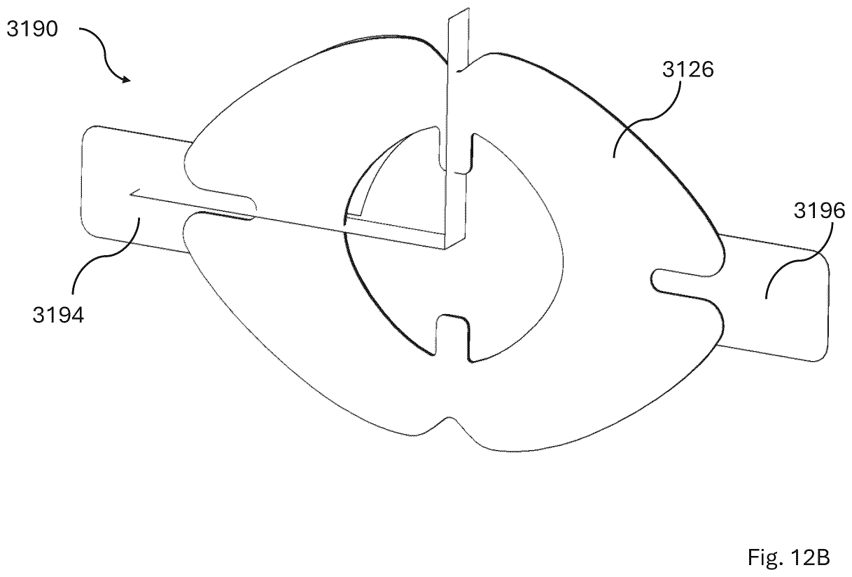

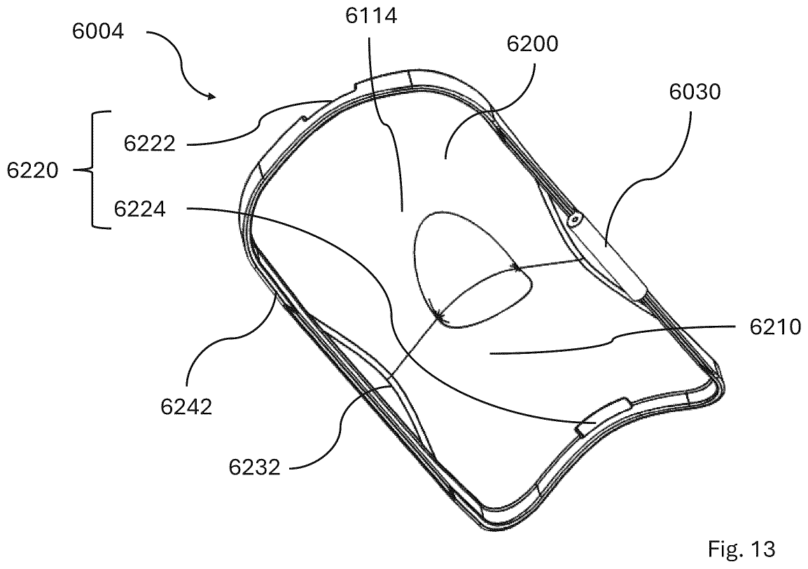

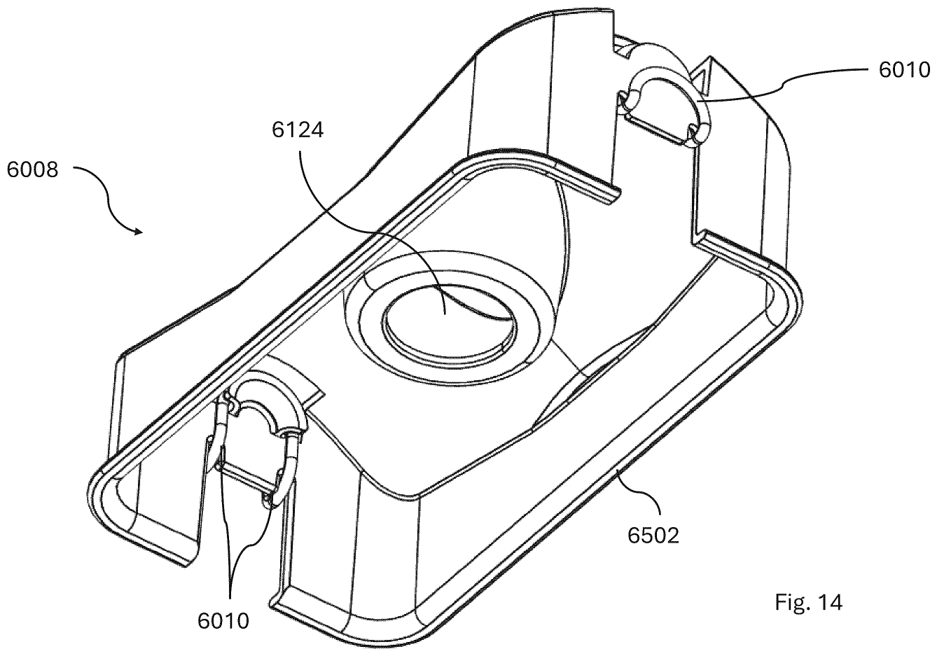

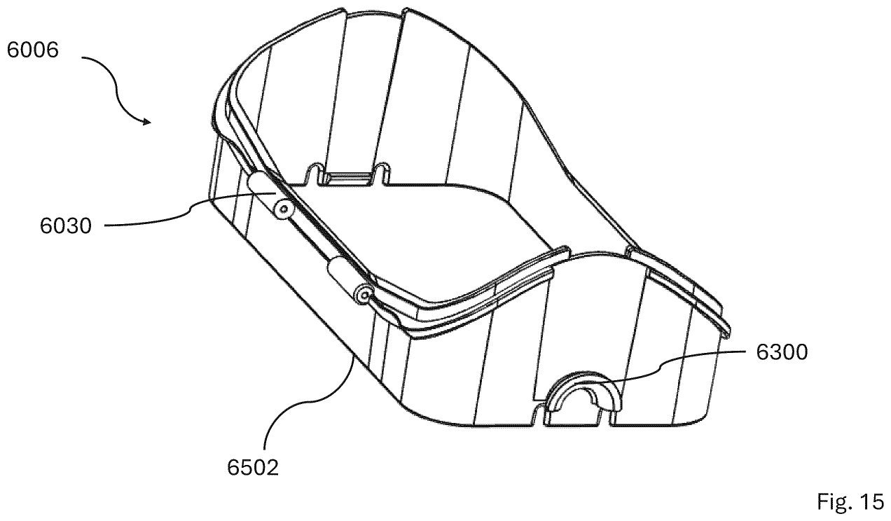

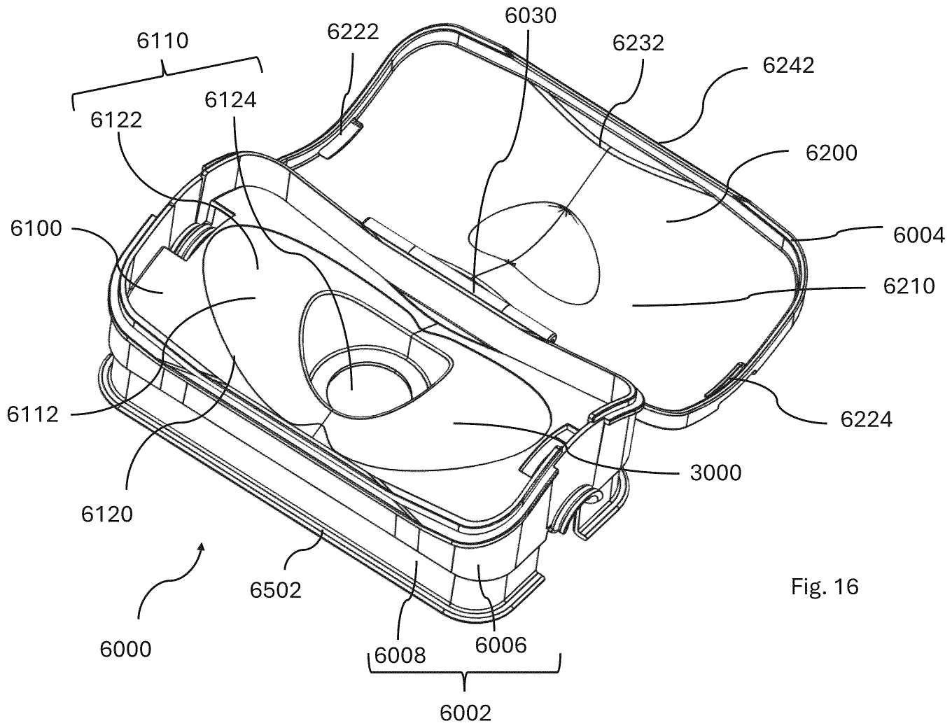

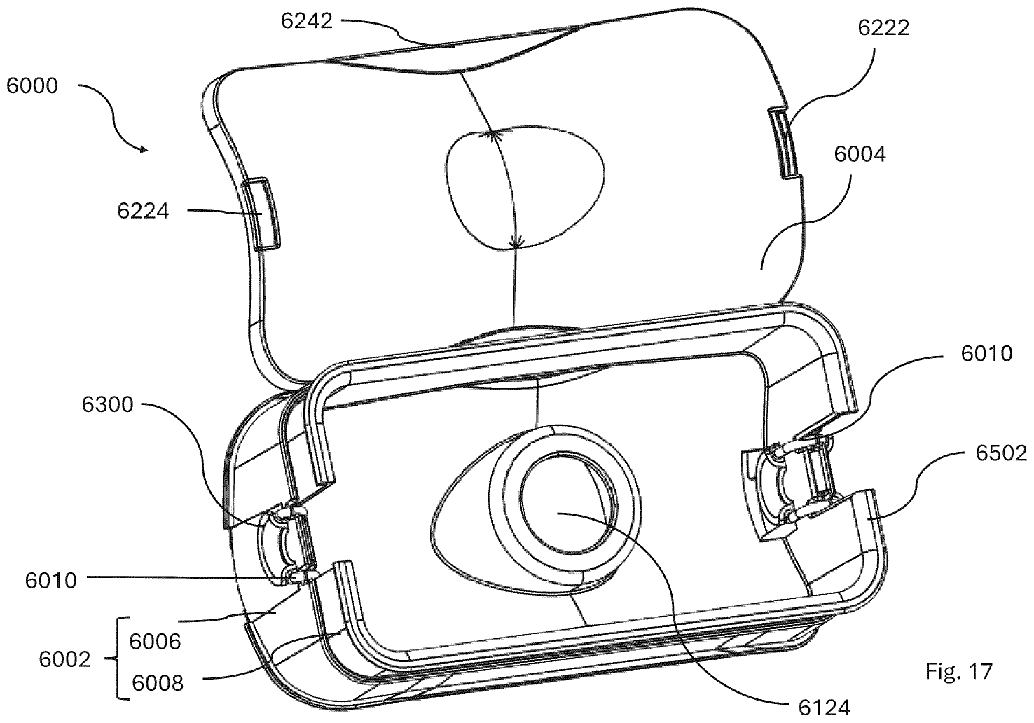

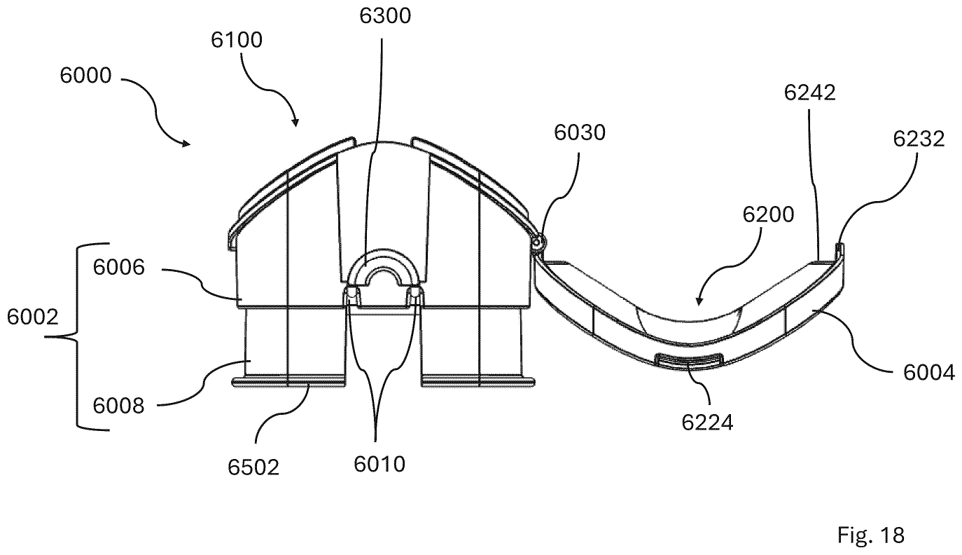

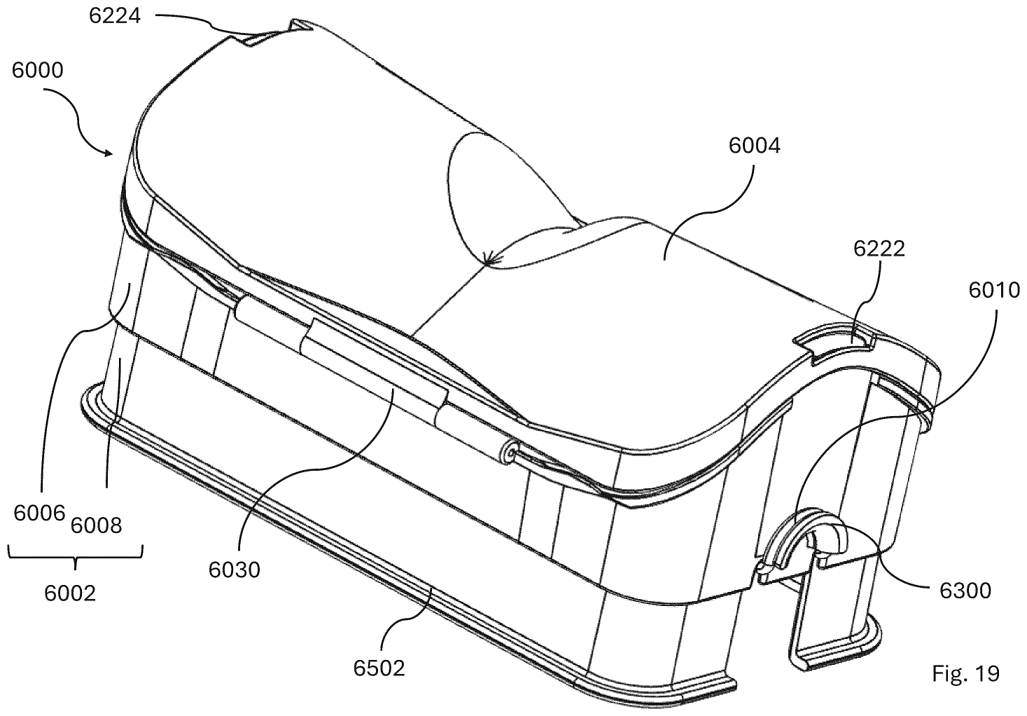

A patient interface 3000, such as shown in FIGS. 3 to 12B, in accordance with certain aspects of the present technology comprises at least some of the following functional aspects: a seal-forming structure 3100, a plenum chamber 3200, a positioning and stabilising structure 3300, and a vent 3400.

In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

The plenum chamber 3200 may be formed of one or more modular components (e.g., a cushion module 3150 together with the seal-forming structure 3100) in the sense that it or they can be replaced with different components, for example components of a different size and/or shape.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH₂O with respect to ambient.

### 4.3.1 Seal-Forming Structure

In one form of the present technology, the patient interface 3000 includes a seal-forming structure 3100 which is configured to form a seal with a region of the patient's face. The seal-forming structure 3100 is thereby configured to secure the plenum chamber 3200 in a sealing engagement with respect to the patient's face. The seal-forming structure 3100 may form an opening to allow a flow of breathable gas to be delivered to at least an entrance to the patient's nares.

In one form of the present technology, the seal-forming structure 3100 provides a target seal-forming region. The target seal-forming region is a region on the seal-forming structure 3100 where sealing may occur. The region where sealing actually occurs-the actual sealing surface-may change within a given treatment session, from day to day, and from patient to patient, depending on a range of factors including for example, where the patient interface was placed on the face, and the shape of a patient's face.

In certain forms of the present technology, the seal-forming structure 3100 is configured so that the shape of the target seal-forming region substantially matches or resembles the shape of the region of the patient's face to which the seal-forming structure 3100 is in use attached, and/or is constructed to be sufficiently flexible that it is able to deform to do so. This may promote a greater degree of sealing against the patient's face and, in the case of a seal-forming structure that adheres to the patient's face, avoids the adhesive surface 3102 pulling on the underlying skin when the patient interface 3000 is in use.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone or a thermoplastic elastomer (TPE).

In certain forms of the present technology, a system is provided comprising more than one a seal-forming structure 3100, each being configured to correspond to a different size and/or shape range. For example, the system may comprise one form of a seal-forming structure 3100 suitable for a large sized head, but not a small sized head and another suitable for a small sized head, but not a large sized head.

More details of seal-forming structure according to certain forms of the technology are described below. Other aspects of seal-forming structures according to forms of the technology are described in more detail in PCT Publication No. WO 2023/015340, the contents of which are herein incorporated by reference.

### 4.3.1.1 Sealing Mechanisms

### 4.3.1.1.1 Adhesive

The seal-forming structure 3100 of certain forms of the technology is configured to adhere, through an adhesive provided on an adhesive surface 3102 of the seal-forming structure 3100, to one or more regions of the patient's face so as to form a seal with a region of the patient's face surrounding an entrance to one or more of the patient's airways. For example, the seal-forming structures 3100 in FIGS. 3 to 12B are each configured to seal around the nasal airways of the patient 1000.

The adhesive-based attachment of the seal-forming structure 3100 to the patient's face allows formation of a highly airtight seal. A high-quality seal improves the effectiveness of positive pressure respiratory therapy since the desired pressure can be maintained in the patient interface. Furthermore, a high quality seal reduces the overall power required to be expended by an RPT device 4000 to maintain the pressure of the breathable gas in the patient interface 3000. When the seal-forming structure 3100 is adhered to the patient's face, another positioning and stabilising structure, e.g. headgear, may not be provided.

### 4.3.1.1.2 Other Sealing Mechanisms

In the forms, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure 3300.

In one form, the seal-forming structure comprises a tension portion. In use, the tension portion is held in tension, e.g. by adjacent regions of the sealing flange.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Sealing Region of Patient's Face

In certain forms of the technology, the seal-forming structure 3100 forms a seal in use with a region of the patient's face surrounding an entrance of the patient's nares. In certain forms, the seal-forming structure 3100 forms a seal around an entrance to the patient's nasal airways (i.e. one or both nares) but not around the patient's mouth.

In the exemplary forms of technology shown in FIGS. 3 to 12B, the seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. This type of patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In some forms, for example as shown in FIGS. 3 to 12B, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

A more detailed description of regions of the patient's face to which a seal-forming structure 3100 seals during use will now be described in the case of certain forms of the technology in which the seal-forming structure is configured to adhere to the patient's face during use.

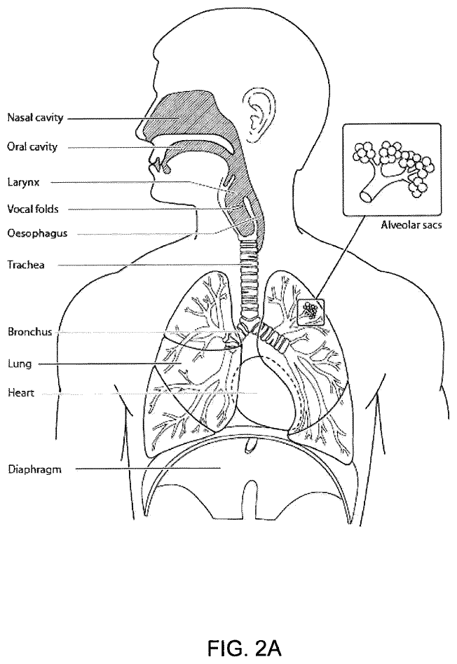

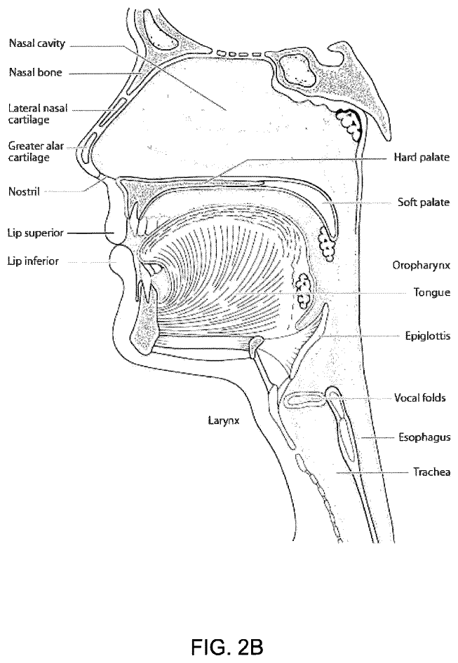

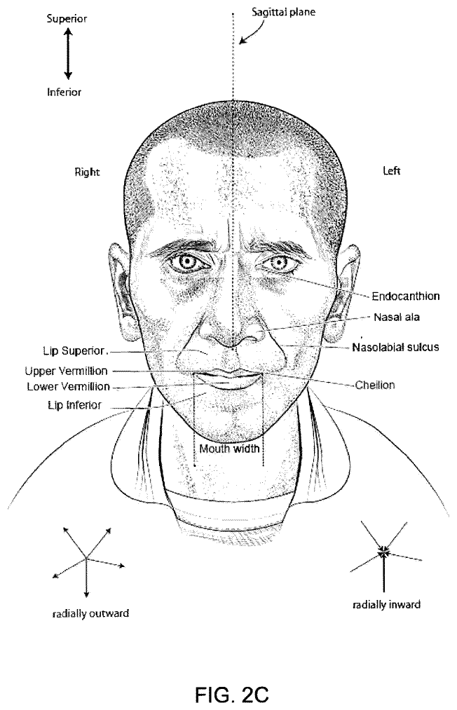

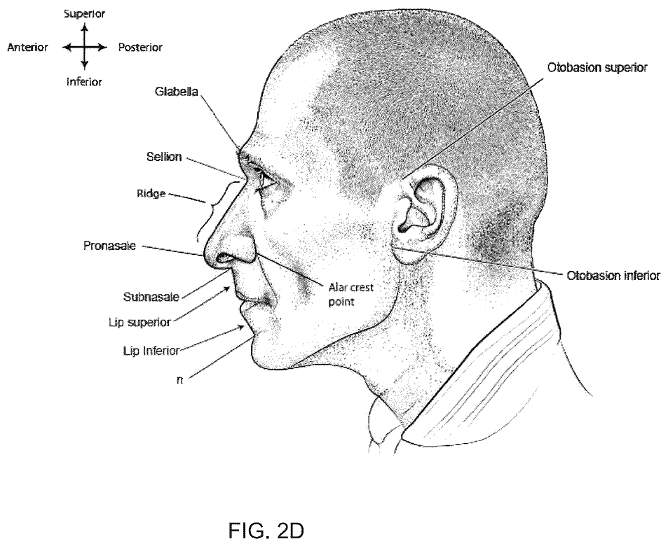

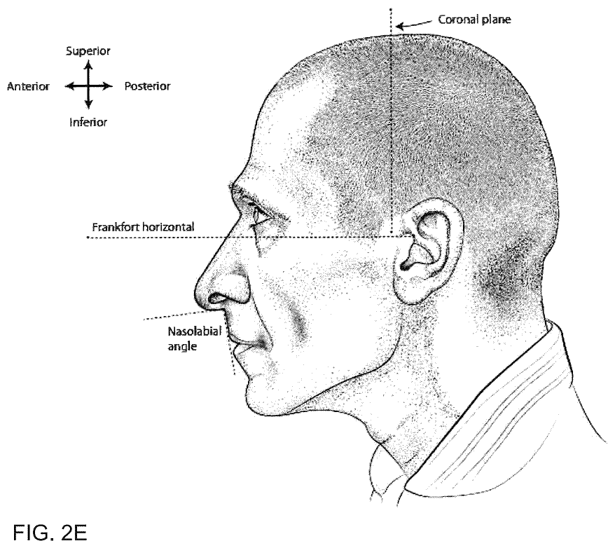

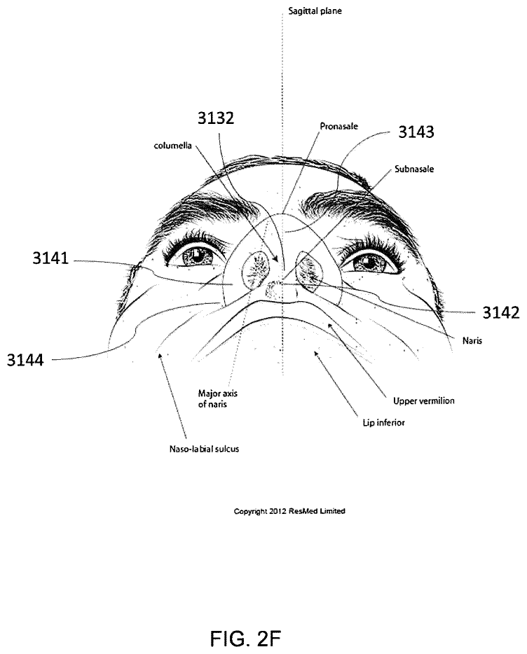

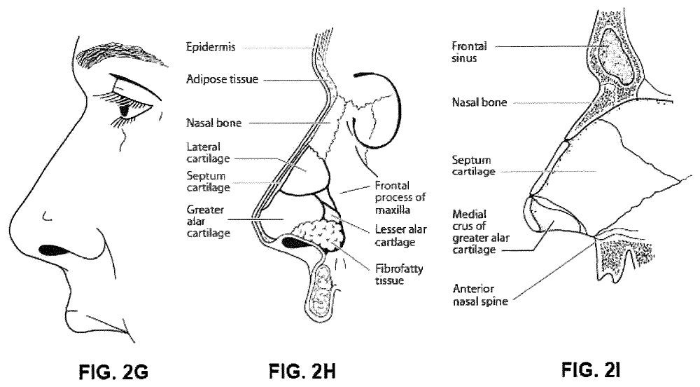

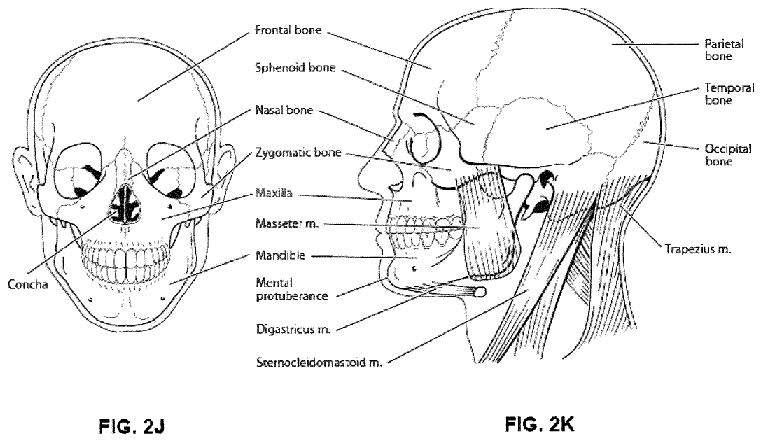

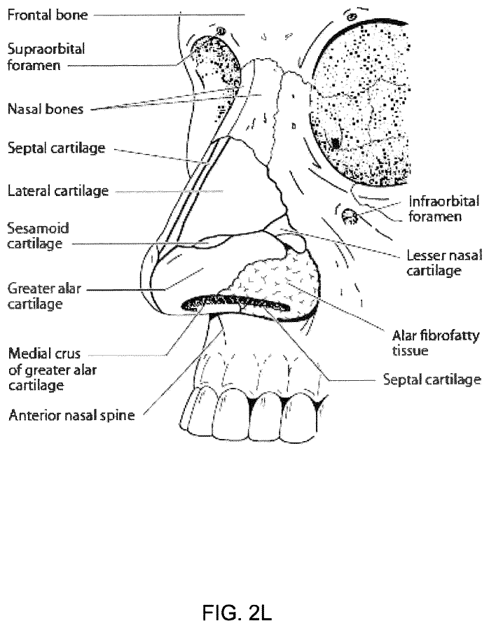

In the form of the technology shown in FIG. 3, the seal-forming structure 3100 is configured to adhere to regions of the patient's face immediately surrounding the nares. These regions may comprise (see FIG. 2F): the alar rim region 3141 (i.e. regions of the ala that are immediately adjacent the nares and may be generally inferiorly facing); the superior-most region of the lip superior 3142, which may comprise the subnasale and/or the region immediately inferior of the subnasale; and an anterior region of the nose that is inferior, e.g. immediately inferior, to the pronasale 3143. In the lateral direction, the seal-forming structure 3100 extends to a region 3144 slightly inferior to the alar crest point, for example a region immediately medial to the junction between the alar crest point and the nasolabial sulcus. In the form of the technology shown, the seal-forming structure 3100 does not adhere to a significant part of the side regions of the nasal alar, although in some forms, or for some faces, it may adhere to the inferior regions of the side regions of the nasal alar. Furthermore, the seal-forming structure 3100 of FIG. 3 does not adhere to the pronasale.

In the form of the technology shown in FIG. 3, the region of the patient's face to which the seal-forming structure 3100 adheres is a band entirely surrounding both the patient's nares. The band may be approximately constant in width around the perimeter of the band.

The region of the face covered by the seal-forming structure 3100 in the form of the technology shown in FIG. 3 has been found not to change shape substantially when a patient 1000 changes their position because this facial region predominantly comprises of cartilage and bone and has relatively little adipose tissue, as compared to the cheek or chin areas. This facial region is also typically free of facial hair or has very little facial hair (for example, some facial hair may be present on the superior-most region of the lip superior 3142). A seal-forming structure 3100 that adheres to this region may be particularly advantageous for a patient 1000 with upper lip hair.

The region of the face covered by the seal-forming structure 3100 in the form of the technology shown in FIG. 3 has also been found to have relatively little variation in shape across patients in representative population samples, including patients of a variety of races.

Further, the size of this region (which may be referred to as the alar rim region) is small in size since it immediately surrounds the nares.

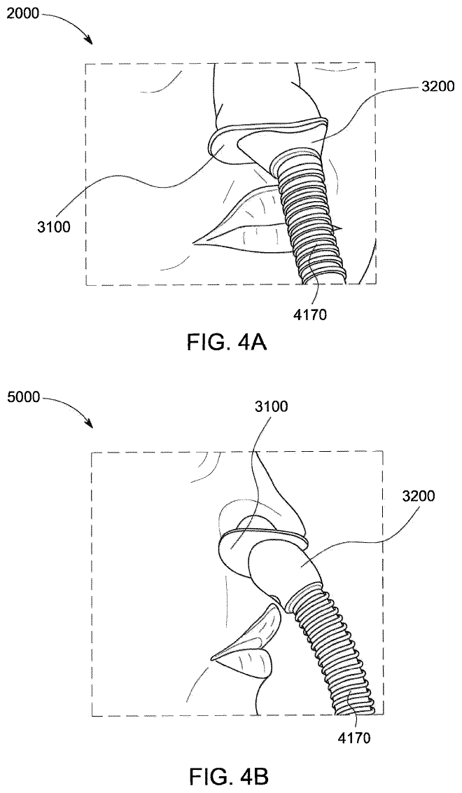

In another exemplary form, as shown in FIGS. 4A and 4B, the seal-forming structure 3100 is configured to adhere to a facial region that extends more superiorly compared to the region shown in FIG. 3 and includes the side regions of the nasal alar. In some forms, the seal-forming structure 3100 is configured to adhere to a side region of the nasal alar and may also extend radially outwardly sufficiently far to adhere in use to regions of the cheeks adjacent the nasal alar crest point, e.g. the regions between the nasal alar and the nasolabial sulcus. This seal-forming structure 3100 may also adhere to the regions of the face to which the seal-forming structure of FIG. 3 adheres. The larger area of adhesion of the seal-forming structure in FIGS. 4A and 4B may improve the amount of adhesion and result in fewer leaks compared to the seal-forming structure of FIG. 3, but may result in more discomfort for the patient.

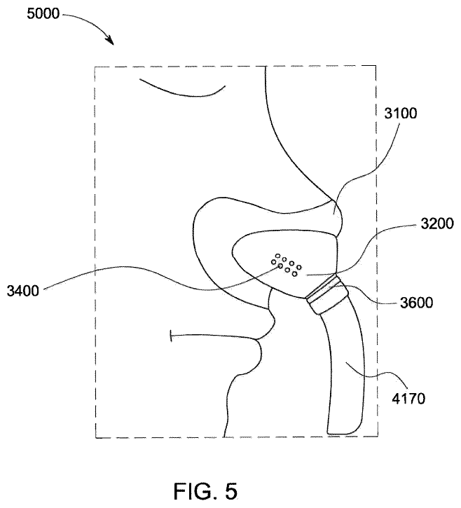

In another exemplary form, as shown in FIG. 5, the seal-forming structure 3100 is configured to adhere to a larger region of the patient's face than the region to which the seal-forming structures of FIGS. 3, 4A and 4B adhere. In this form the seal-forming structure adheres to a region that extends in a superior direction up to the alar crease region, i.e. when the seal-forming structure is adhered to the patient's face, a superior-most portion of the seal-forming structure adheres to the patient's alar crease. The seal-forming structure 3100 in this form may additionally, or alternatively, adhere to a substantial portion of the lip superior. Furthermore, the seal-forming structure 3100 in this form may additionally, or alternatively, adhere to the pronasale region, which may include a point which is immediately superior to the pronasale. In the lateral direction, the seal-forming structure 3100 in this form adheres to a region of the patient's cheeks adjacent to the nasal alar, including the regions between the nasal alar and the nasolabial sulcus, and the seal-forming structure 3100 may cover the nasal alar completely in use. This seal-forming structure 3100 may also adhere to the regions of the face to which the seal-forming structures of FIGS. 3, 4A and/or 4B adhere. Alternatively, the seal-forming structure of FIG. 5 may be configured to adhere to some regions positioned radially outwardly from the nares compared to some regions to which the seal-forming structures of FIGS. 3, 4A and/or 4B adhere.

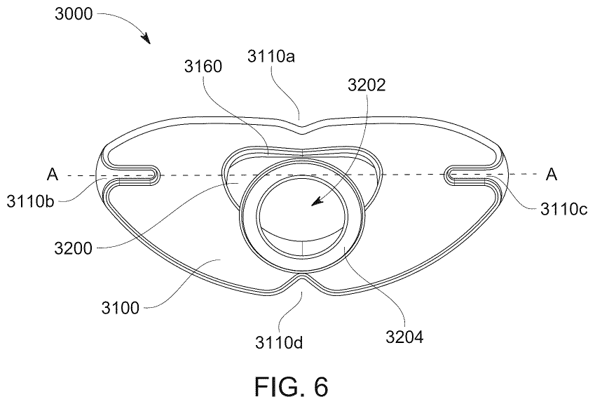

The forms of the technology shown in FIGS. 6 to 12A are also configured to seal to regions of the patient's face around and proximate the patient's nares.

### 4.3.1.3 Composition of the Seal-Forming Structure

In certain forms of the present technology, the seal-forming structure 3100 is constructed from a material having one or more of the following properties: biocompatibility; soft; flexible; stretchable and optionally resilient. In exemplary forms of the technology, the seal-forming structure 3100 is formed from silicone or a thermoplastic elastomer (TPE). In other forms, the seal-forming structure 3100 is formed from a textile, fabric and/or foam material.

In the case of forms of the technology in which an adhesive is used to adhere the seal-forming structure 3100 to the patient's face, any form of adhesive may be used, and adhesives may be applied to any suitable substrate, which may include materials such as mentioned above, for example silicone or TPE. For example, a rubber zinc oxide adhesive may be used. In other forms, other adhesives may be used, for example acrylic or acrylate adhesives, or silicone adhesives.

The adhesives may be provided in the form of an adhesive layer 3190, for example an adhesive tape, in which the adhesive is already provided on a substrate (i.e. the tape) to form the adhesive surface 3102, which may be advantageously used as the seal-forming structure 3100, or part thereof, or may be readily attached to the seal-forming structure 3100. Examples of suitable tapes are the 3M™ Nexcare™ tape and Leukoplast tape. In some forms, the material may comprise a rayon substrate to which an adhesive is applied. In one form, the seal-forming structure may be formed from, or may comprise, 3M™ Product No. 2484(which uses a silicone adhesive, “Hi-Tack 3M medical silicone adhesive”), 3M™ Medical tape 9833 (which uses an acrylic/acrylate adhesive), or a similar type of product or a product having a similar structure. Multiple layers of a tape or product may be used to form the seal-forming structure 3100.

In the forms of the technology shown in FIGS. 6 to 9, the seal-forming structure 3100 may comprise a flange 3105 connected to the patient-facing (or posterior) side of the plenum chamber 3200. The flange 3105 may extend radially outwards from the opening in the patient-facing side of the plenum chamber 3200 in all directions. The flange 3105 may be formed from silicone or TPE, for example, and in some forms may be formed from the same material as is used to form the plenum chamber 3200, for example the flange 3105 may be integrally formed with the plenum chamber 3200. In these forms, the adhesive may be applied to the patient-facing side of the flange 3105. In these forms, the entirety of, or a substantial part of, the adhesive surface 3102 of the seal-forming structure 3100 may be on the patient-facing side of the flange 3105, or in alignment with it. For example, in these forms, the adhesive surface 3102 may not extend radially outwardly from the flange 3105, as contrasted with other forms described later.

In some forms, for example as shown in FIGS. 8 to 12A, the adhesive may be carried by a patient-facing side of the flange 3105 and may be provided on one or more adhesive layers 3190. Each layer 3190 may cover a substantial proportion of the patient-facing side of the flange 3105, for example substantially all of the patient-facing side of the flange 3105.

In some forms, for example as shown in FIGS. 6, 8 and 9, the flange 3105 may be formed as a single component, which in some forms may be integrally formed with the plenum chamber 3200. In other forms, for example the forms of technology illustrated in FIGS. 10 to 12A, the flange 3105 may be formed from an assembly of two or more components, for example a first flange region integrally connected to the plenum chamber 3200 and a second flange region formed from a different material, which may be comprised as part of an adhesive layer 3190. In other forms, for example as shown in FIG. 11, the flange 3105 may be formed entirely from the adhesive layer 3190.

Each adhesive layer 3190 may be formed with a hole in it, for example in a central region. The hole may be sized and shaped to substantially match the opening in the seal-forming structure 3100 and the opening in the patient-facing side of the plenum chamber 3200 so as to align with these openings when the patient interface 3000 is assembled and to allow the flow of breathable gas through the openings and hole to the patient's airways. Each adhesive layer 3190 may further be formed with notches 3110 as described below.

In some forms, each adhesive layer 3190 is formed from a double-sided adhesive tape, i.e. a tape which has adhesive on both surfaces of a substrate. The double-sided nature of the tape may be achieved by the tape being provided in this form from a supplier, or through the application of additional adhesive to the non-adhesive side of an originally single-sided adhesive tape.

In the form shown in FIG. 8, the seal-forming structure 3100 comprises a single layer 3190 of double-sided adhesive tape. The non-patient-facing side of this layer 3190 is adhered to the patient-facing side of the flange 3105. The patient interface 3000 may comprise a patient-facing removable layer 3120 which is configured to cover the patient-facing side of the adhesive layer 3190 until the patient is ready to adhere the seal-forming structure 3100 to their face. The patient-facing removable layer 3120 may be removed from the adhesive layer 3190 prior to the seal-forming structure 3100 being adhered to the face. The patient may be provided with replacement portions of adhesive layer 3190 to replace on the flange 3105 each use of the patient interface 3000 or every few uses.

In the form shown in FIG. 10, the seal-forming structure 3100 comprises a single layer 3190 of single-sided adhesive tape. The seal-forming structure 3100 may comprise a ring of adhesive material 3122 for adhering the plenum chamber 3200 to the seal-forming structure 3100. The ring of adhesive material 3122 may be located on a non-patient-facing side of the layer 3190 and around the opening hole in the seal-forming structure 3100 through which the breathable gas is conveyed to the patient in use. Again, the patient may be provided with replacement portions of adhesive layer 3190 to replace each use of the patient interface 3000 or every few uses.

In other forms, for example as shown in FIG. 9, the seal-forming structure 3100 comprises multiple adhesive layers 3190, for example two layers formed from layers 3190a and 3190b. When the seal-forming structure 3100 is assembled, the non-patient-facing side of the first layer 3190a may be adhered to the patient-facing side of the flange 3105, the non-patient-facing side of the second layer 3190b may be adhered to the patient-facing side of the first layer 3190a and the patient-facing side of the second layer 3190b may be adhered to the patient's face when the patient interface 3000 is in use. Prior to assembly of the seal-forming structure 3100, the non-patient-facing side of the first layer 3190a may be covered by a non-patient-facing removable layer 3180 to protect the adhesive before assembly and the patient-facing side of the second layer 3190b may be covered by a patient-facing removable layer 3120 to protect the adhesive before assembly. This layered assembly of multiple layers (for example layers 3180, 3190a, 3190b and 3120) may be supplied separately to the plenum chamber 3200. In addition, multiple such layered assemblies may be provided and the patient may replace the layered assemblies for different uses of the patient interface 3000, for example the layered assembly may be replaced each night or every few nights.

In some forms, the second layer 3190b may be a tape particularly suited to adhering to a patient's skin, for example medical tape, while the first layer 3190a may be more suitable for adhering to the flange 3105. In some forms, the medical tape used as the second layer 3190b may not be double-sided and therefore a double-sided first layer 3190a may be used to adhere the medical tape to the plenum chamber 3200.

In the case of the form shown in FIG. 9, each adhesive layer 3190a and 3190b may be sized and shaped similarly to the size and shape of the patient-facing side of the flange 3105 so that each layer 3190 covers a substantial part of the patient-facing area of the flange 3105 but does not extend radially outwardly from the edges of the flange.

In some forms, a fluid adhesive may be applied to a surface of the seal-forming structure 3100, for example in the form of a spray.

### 4.3.1.4 Shape Retainer

In certain forms of the technology, the patient interface 3000 may comprise one or more shape retainers 3170. The shape retainer(s) may be configured to promote retention of the shape of the seal-forming structure 3100 before the seal-forming structure is made to adhere to the patient's face, for example to a sufficient extent to prevent the seal-forming structure 3100 from crumpling, folding or sagging in a way that makes it difficult for the patient 1000 to affix the seal-forming structure 3100 to their face.

The one or more shape retainers 3170 may be one or more components, an assembly or a structure which are formed with a shape and/or out of materials to provide a predetermined level of stiffness suitable to promote the desired level of retention of the shape of the seal-forming structure 3100. In certain forms, the one or more shape retainers 3170 are comprised as part of the seal-forming structure 3100. In other forms, the shape retainer(s) 3170 may be attached to the seal-forming structure 3100 to promote retention of the shape of the seal-forming structure 3100, for example by stiffening one or more regions of the seal-forming structure 3100.

In certain forms, the plenum chamber 3200, which may be formed so as to be more rigid than the seal-forming structure 3100, and to which the seal-forming structure 3100 is provided, may act to help retain the shape of the seal-forming structure 3100. The region of the seal-forming structure 3100 connected to the plenum chamber 3200 may be maintained in its shape by the relatively rigid plenum chamber 3200, and this may also help maintain the shape of other parts of the seal-forming structure 3100.

In other forms, for example the form shown in FIGS. 6 to 9, the removable layer 3120 may act to help retain the shape of the seal-forming structure 3100 prior to removing the removable layer 3120. To act in this way, the removable layer 3120 may be formed so as to be relatively rigid in comparison to the seal-forming structure 3100, for example the removable layer 3120 may be formed from a material and/or in a shape that is relatively rigid.

The exemplary patient interfaces 3000 illustrated in FIGS. 10 to 12A are other forms of the technology in which the patient interface 3000 comprises at least one shape retainer 3170 which is configured to promote retention of the shape of the seal-forming structure 3100 before the seal-forming structure 3100 is made to adhere to the patient's face. In these forms, the shape retainer 3170 comprises a loop which extends around a substantial part of the outer perimeter of the seal-forming structure 3100. The shape retainer 3170 acts to provide shape retention to the radially outer regions of the seal-forming structure 3100, and thereby acts to promote all of the seal-forming structure 3100 to retain its shape until the shape retainer 3170 is removed.

The shape retainers 3170 of FIGS. 10 to 12A may be formed from a material and/or having a shape so as to make the shape retainer 3170 more rigid than the seal-forming structure 3100. For example, the shape retainer 3170 may be formed from a material that is thicker than the material used to form the seal-forming structure 3100. Additionally, or alternatively, the shape retainer 3170 may be formed from a material that is harder than the material used to form the seal-forming structure 3100. In some exemplary forms, the shape retainer 3170 may be provided with an additional rigidising structure to provide additional rigidity, for example rigidising ribs. The shape retainer 3170 may not be so rigid that its shape cannot be altered by the patient so that the patient is still able to flex the shape retainer 3170 and seal-forming structure 3100 when adhering the patient interface 3000 to their face. In this regard, the shape retainer 3170 may be described as semi-rigid. In one example, the shape retainer 3170 may be formed from paper, for example kraft paper.

In the forms of the technology illustrated in FIGS. 10 to 12A, the shape retainer 3170 is configured to be positioned on a non-patient-facing side of the seal-forming structure 3100. For example, the shape retainer 3170 may be adhered to the non-patient-facing side of the seal-forming structure 3100. In some forms, an adhesive may be used to adhere the shape retainer 3170 to the seal-forming structure 3100. In such forms, the adhesive may be applied to the patient-facing side of the shape retainer 3170 or to the non-patient-facing side of the seal-forming structure 3100, or both. The strength of the adhesive may be relatively low to enable the shape retainer 3170 to be easily removed from the seal-forming structure 3100 by the patient. In other forms, the shape retainer 3170 may be weakly held in place relative to the seal-forming structure 3100 through a natural tackiness between the seal-forming structure 3100 and the shape retainer 3170. An advantage of the shape retainer 3170 being provided to a non-patient-facing side of the seal-forming structure 3100 is that the patient is able to position the seal-forming structure 3100 on their face while the shape retainer 3170 is still in position. Then, when the seal-forming structure 3100 is adhered to the face, the shape retainer 3170 may be removed, as has occurred in the example of FIG. 11.

In other forms, the shape retainer 3170 may be positioned on a patient-facing side of the seal-forming structure 3100. The mechanism of retention in these forms may be the same or similar to as explained above for forms in which the shape retainer 3170 is positioned on a non-patient-facing side of the seal-forming structure 3100. An advantage of such forms is that the same adhesive surface of the seal-forming structure 3100 used to adhere the seal-forming structure 3100 to the patient's face may be used to retain the shape retainer 3170 in position before its removal.

In the forms shown in FIGS. 10 to 12A, the shape retainer 3170 comprises a loop which extends around a substantial part of the outer perimeter of the seal-forming structure 3100. In some forms, the shape retainer 3170 is positioned around the radially outermost portions of the seal-forming structure 3100 while in other forms the shape retainer 3170 is positioned in a loop around radially outer portions of the seal-forming structure 3100, but may not necessarily be provided to the outermost portions all around the perimeter of the seal-forming structure 3100.

Being in the form of a loop, the shape retainer 3170 forms a hole radially inside the loop. In addition, when the shape retainer 3170 is mounted to the seal-forming structure 3100, there may be regions of the non-patient-facing side of the seal-forming structure 3100 that are not covered by the shape retainer 3170, i.e. there may be a gap between the patient-proximal periphery of the plenum chamber 3200 and the radially inner edge of the shape retainer 3170. It has been found that a shape retainer in this form may be more advantageous than a shape retainer 3170 that covers all or a significant proportion of the non-patient-facing side of the seal-forming structure 3100 because, in the latter case, the shape retainer 3170 may provide too much rigidity to the seal-forming structure 3100 so that it becomes difficult to apply to the face.

In the exemplary forms shown in FIGS. 10 and 12A, the patient interface 3000 may comprise a ring of adhesive material 3122 on a non-patient facing side for adhering the plenum chamber 3200 to the seal-forming structure 3100. The ring of adhesive material 3122 may be covered by a non-patient-facing removable layer 3180 to protect the adhesive before the plenum chamber 3200 is brought into contact with the ring of adhesive material 3122. In this form, there is an annular gap between the ring of adhesive material 3122 and the shape retainer 3170 and an annular region of the non-patient-facing side of the seal-forming structure 3100 may be uncovered by the shape retainer 3170 due to this gap.

The shape retainer 3170 may have a constant radial thickness around the loop or, as shown in FIGS. 10 and 12A, the radial thickness of the shape retainer 3170 may vary around the loop. The radial thickness of the shape retainer 3170 may be thicker in regions that would benefit from a greater amount of shape retention. For example, the lateral regions of the shape retainer 3170 that are provided to lateral regions of the seal-forming structure 3100 (from the perspective of when the seal-forming structure 3100 is in position on the patient's face) may be radially thicker than other regions, for example thicker than inferior and superior medial regions of the shape retainer 3170. The lateral regions of the seal-forming structure 3100 may benefit from more shape retention in the illustrated form because these regions have a larger area than the inferior and superior medial regions of the seal-forming structure 3100 and therefore may be more liable to flop or crumple when the patient is adhering the seal-forming structure 3100 to their face.

### 4.3.1.5 Non-Patient-Facing Removable Layer

It has already been explained that, in certain forms of the technology, the seal-forming structure 3100 may comprise a non-patient-facing removable layer 3180 to protect an adhesive on a non-patient-facing side of a layer of the seal-forming structure, for example layer 3190, before adhering the layer to the patient-facing side of the flange 3105 or to the plenum chamber 3200. Exemplary forms of the non-patient-facing removable layer 3180 are illustrated in FIGS. 9 and 12A. The non-patient-facing removable layer 3180 may alternatively be referred to as a “release liner”.

In the example of FIG. 12A, the non-patient-facing side of the layer 3190 may carry a ring of adhesive material 3122 for adhering the layer 3190 to the flange 3105 or to the plenum chamber 3200. The ring of adhesive material 3122 may be covered by the non-patient-facing removable layer 3180 to protect the adhesive before the plenum chamber 3200 is brought into contact with the ring of adhesive material 3122. In such examples, the non-patient-facing removable layer 3180 may be similarly ring-shaped so as to entirely cover the ring of adhesive material 3122 but it may not cover other regions of the non-patient-facing side of the tape 3190. That is, the non-patient-facing removable layer 3180 may be formed with a hole therein. When the non-patient-facing removable layer 3180 is positioned to cover the ring of adhesive material 3122, the hole may substantially align with the hole in the tape 3190, i.e. the opening in the seal-forming structure 3100 through which breathable gas is delivered to the patient when the patient interface 3000 is in use.

### 4.3.2 Plenum Chamber

The plenum chamber 3200 of certain forms of the technology is configured to receive the flow of breathable gas at the therapeutic pressure for breathing by the patient from the air circuit 4170. The plenum chamber may be formed to be pressurisable to a therapeutic pressure of at least 6 cmH₂O above ambient air pressure, and up to pressures of around 20 cmH₂O or 30 cmH₂O in certain forms.

In one form, the plenum chamber 3200 has a perimeter that is shaped to be complementary to the surface contour of the face of an average person in the region where a seal will form in use. The complementary shape of the perimeter of the plenum chamber 3200 may be configured to facilitate correct positioning of the patient interface 3000 against the patient's face in use.

Alternatively, in certain forms, the plenum chamber 3200 may be shaped in a customised way to an individual patient. Alternatively, the plenum chamber 3200 of a patient interface 3000 may be selected from one of a plurality of possible forms of plenum chamber 3200, with the appropriate plenum chamber for an individual patient being selected as being most suitable for them.

In use, a marginal edge of the plenum chamber 3200 is positioned in close proximity to an adjacent surface of the face. Actual contact with the face may be provided by the seal-forming structure 3100. The seal-forming structure 3100 may extend in use about the entire perimeter of the plenum chamber 3200.

The plenum chamber 3200 may include at least two openings. One opening, which may be formed in a patient-facing, or posterior, side of the plenum chamber 3200, allows pressurised gas to flow from the internal volume of the plenum chamber 3200 to the patient's airways through the seal-forming structure 3100. This opening may also allow exhaled gas from the patient to flow into the plenum chamber 3200. Another opening, which may be referred to as the plenum chamber inlet port 3202, is configured to allow the flow of breathable gas from the air circuit 4170 into the plenum chamber 3200. In certain forms, the plenum chamber inlet port 3202 may be disposed on a side of the plenum chamber 3200 facing away from the patient in use, i.e. an anterior side of the plenum chamber 3200. In other forms, the patient interface may comprise one or more plenum chamber inlet ports 3202 disposed on lateral sides (e.g. left and right sides) of the plenum chamber 3200.

In certain forms, for example in the forms shown in FIGS. 6 to 11, the plenum chamber 3200 may be configured such that the opening in the patient-facing side of the plenum chamber 3200 is sized and shaped to cover both the patient's nares when the patient interface 3000 is in use. In these forms, the size and shape of the opening may approximately match, and be positioned adjacent in use, an area of the underside of the patient's nose that is made up of the patient's nares and the patient's columella (see FIG. 2F). The opening may be formed by a rim 3210 on the posterior side of the plenum chamber 3200 and the plenum chamber 3200 may be configured so that, in use, the rim 3210 is positioned adjacent and anterior to lateral, posterior and anterior edges of the patient's nares.

The posterior side of the plenum chamber 3200 may be shaped to be complementary to the shape of the underside of the patient's nose, against which the plenum chamber 3200 may be positioned in use. In the forms shown in FIGS. 6 to 11, the posterior side of the plenum chamber 3200 may be formed from the rim 3210 around the opening on the patient-facing side of the plenum chamber 3200. In certain examples, the posterior side of the plenum chamber 3200 may lie on a saddle-shaped surface, where in this context “saddle-shaped” refers to a geometrical surface in which lines on the surface are convex in one direction and concave in another direction, orthogonal to the first direction. More particularly, the posterior side of the plenum chamber 3200 may be concave in the lateral direction relative to the patient's face so that the patient's columella extends into the trough of the concavity, as shown in FIG. 11. The posterior side of the plenum chamber 3200 may be convex in the anterior-posterior direction so that the peaks of the convexity extend towards lateral edges of the patient's nares, again as shown in FIG. 11.

When the seal-forming structure 3100 is assembled with the plenum chamber 3200, the seal-forming structure 3100, which may be formed from a flexible material, may be caused to deform to a shape that is similar to the shape of the plenum chamber 3200. As illustrated, for example in FIGS. 6 to 11, the seal-forming structure 3100 may, prior to being brought into sealing contact with the patient's face, have a saddle-shaped patient-facing surface.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

In some forms, the plenum chamber 3200 and the seal-forming structure 3100 are formed from a single homogeneous piece of material, for example silicone or TPE.

### 4.3.3 Connection of Plenum Chamber and Seal-Forming Structure

The plenum chamber 3200 may be connected to the seal-forming structure 3100. In certain forms, the plenum chamber 3200 is directly connected to the seal-forming structure 3100. For example, the plenum chamber 3200 may be connected to the seal-forming structure 3100 through a mechanical joint, with adhesive or the plenum chamber 3200 and the seal-forming structure 3100 may be integrally formed. In other forms, the plenum chamber 3200 may be indirectly connected to the seal-forming structure 3100, for example via another component.

### 4.3.3.1 Adhesive Connection

In some forms, two portions of the patient interface 3000 may be adhered together with adhesive. For example, in some forms, such as the forms illustrated in FIGS. 10 to 12A, the plenum chamber 3200 may be connected to the seal-forming structure 3100 with adhesive. It has already been described above that an adhesive on a non-patient-facing side of the seal-forming structure 3100 may adhere to a patient-facing side of the plenum chamber 3200, for example rim 3210. One or more adhesive layers 3190 may be positioned between the non-patient-facing side of the seal-forming structure 3100 and the plenum chamber 3200 to adhere the two components together. Alternatively, a first flange region may be adhered to a second flange region. In some forms, the one or more adhesive layers 3190 may be formed on the same component as the adhesive layers that adhere the seal-forming structure 3100 to the patient's face.

### 4.3.4 Vent

In certain forms of the technology, the patient interface 3000 comprises a vent 3400 constructed and arranged to allow for the washout of exhaled gases, e.g. carbon dioxide. The vent 3400 may be implemented through a vent structure, which may be formed or provided in any one or more components of the patient interface 3000.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 5 to about 80 holes, or about 10 to about 40 holes, or about 20 to about 25 holes.

In certain forms of the technology, for example as shown in FIG. 5, the vent 3400 may be located in the plenum chamber 3200.

Alternatively, the vent 3400 may be located in the air circuit 4170 that delivers the flow of breathable gas from the RPT device 4000 to the plenum chamber 3200, for example in a part of the air circuit 4170 located proximate to the plenum chamber 3200.

### 4.3.4.1 Ports

In certain forms of the present technology, a patient interface 3000 includes one or more ports that allow access to the volume within the plenum chamber 3200. In certain forms this allows a clinician to supply supplementary oxygen. In one form, this allows for the direct measurement of a property of gases within the plenum chamber 3200, such as the pressure.

### 4.3.4.2 Breath-To-Atmosphere Vent

In certain forms of the technology, the patient interface 3000 may include a vent 3400 configured to be able to adopt at least two configurations. In one configuration, which may be termed an open configuration, the vent 3400 allows the patient to inhale and exhale through the vent 3400 without significant impedance, or with a level of impedance that is largely unnoticeable by the patient. In another configuration, which may be termed a closed configuration, the vent 3400 is more occluded than in the open configuration. In some forms, in the closed configuration, the vent 3400 allows the washout of exhaled gases from an interior of the plenum chamber 3200 to ambient whilst substantially maintaining the pressure within the plenum chamber as positive with respect to ambient. In other forms, in the closed configuration, the vent may substantially block all washout of gases through the vent, and instead exhaled gases exhaust through a separate vent structure. Such a vent 3400 may be referred to as a “breathe-to-atmosphere” vent (BTA vent).

Whether the BTA vent adopts the open or closed configuration may be based on the pressure of the supply of breathable gas provided from the RPT device 4000 to the patient interface 3000. When no breathable gas is supplied, or when the flow of breathable gas is supplied at a pressure below a certain threshold, for example below a therapeutic pressure level such as 6 cmH₂O, the BTA vent may be configured to adopt the open configuration. When the flow of breathable gas is supplied at a pressure above a certain threshold, for example above a therapeutic pressure level such as 6 cmH₂O, the BTA vent may be configured to adopt the closed configuration.

A further explanation of a patient interface system comprising a vent that may be considered to act in the manner of a BTA vent, as described above, is provided in PCT Application No. PCT/US2012/055148, the contents of which are hereby incorporated by reference.

In one application, a BTA vent may be used in a patient interface system in which the BTA vent is configured to adopt the open configuration when the patient first dons the patient interface 3000 and while the patient is detected as being awake by the RPT device 4000. In this configuration, the RPT device may not supply a flow of breathable gas, or may be configured to provide a small flow of breathable gas to help flush out exhaled CO₂ from the plenum chamber 3200. Once the RPT device 4000 detects that the patient has gone to sleep, the flow of breathable gas may be supplied at a therapeutic pressure, which causes the BTA vent to adopt the closed configuration.

### 4.3.4.2.1 Anti-Asphyxia Valve

One form of BTA vent is an anti-asphyxia valve (AAV) which is conventionally used in patient interfaces which cover both the nose and mouth as a measure to mitigate the risk of asphyxiation. The AAV ensures ventilation to the airways of the patient 1000 in case of disruption of the supply of breathable gas to the plenum chamber 3200 and/or the airways of the patient 1000. In certain forms of the present technology, a patient interface 3000 may comprise a conventional design of AAV acting in use as a BTA vent, as described above.

### 4.4 ASSEMBLY APPARATUS

In certain forms of the technology, there is provided an assembly apparatus 6000 to assist in assembling the patient interface 3000. In particular, the assembly apparatus 6000 may be used to adhere two portions of the patient interface 3000 together, for example to apply an adhesive layer 3190 to the patient interface 3000 or to adhere a seal-forming structure 3100 to a plenum chamber 3200. As explained earlier, there may be a need to assemble the patient interface 3000, or to refresh the adhesive on the patient interface 3000 that adheres the patient interface 3000 to the patient's face, and this may be difficult to perform effectively. An assembly apparatus 6000 according to forms of the technology may help to achieve this.

Three exemplary forms of assembly apparatus 6000 are illustrated in FIGS. 13 to 47. These are illustrative examples only and other forms of the technology may also be provided, as per the description below.

### 4.4.1 Assembly Components

In certain forms of the technology, for example as illustrated in FIGS. 13 to 47, the assembly apparatus 6000 may comprise a first assembly component 6002 and a second assembly component 6004. The assembly components 6002 and 6004 are configured to receive separate portions of the patient interface 3000 and are then brought together in order to assemble the two portions together. Using the assembly apparatus 6000 to perform this task may make the assembly process easier, for example by helping to align the portions for correct assembly. The apparatus 6000 may also help the assembly process be consistently repeatable for correct assembly multiple times.

For example, the first assembly component 6002 may be configured to receive a first portion of the patient interface 3000 and the second assembly component 6004 may be configured to receive a second portion of the patient interface 3000. It should be understood that, unless the context clearly requires otherwise, one of the assembly components “receiving” one of the portions of the patient interface refers to at least some part of that portion being received by the respective assembly component. The part of the portion that is received may be a small part of the portion, or it may be a substantial part. A portion may be received by coming into engagement with, or otherwise being physically associated with, the respective assembly component.

The first and second portions of the patient interface 3000 that are adhered together through use of the assembly apparatus 6000 to assemble the patient interface 3000 may be any two components or sub-assemblies of components forming the patient interface 3000 that are adhered together. The second portion of the patient interface 3000 may comprise a first adhesive surface on a patient-facing side of the second portion and a second adhesive surface on a non-patient-facing side of the second portion. The first adhesive surface may be configured to adhere the patient interface to the patient's face and the second adhesive surface may be configured to adhere the second portion to the first portion. In certain forms, for example in the case of the assembly apparatus 6000 shown in FIG. 8, the first portion may be a sub-assembly comprised of the plenum chamber 3200 and seal-forming structure 3100 and the second portion may be an adhesive layer 3190 having a non-patient-facing adhesive surface and a patient-facing adhesive surface. In use, the adhesive layer 3190 may be applied to the seal-forming structure 3100 of the patient interface 3000 when the first assembly component 6002 engages with the second assembly component 6004, for example when the assembly components 6002, 6004 are brought together. In other forms, for example in the case of the form of FIG. 10, the first portion may be the plenum chamber 3200 and the second portion may be at least a portion of the seal-forming structure 3100. The plenum chamber 3200 may be connected to, for example integrally formed with, a first flange region. The seal-forming structure 3100 may comprise the ring of adhesive material 3122 and/or the adhesive layer 3190. In use, the ring of adhesive material 3122 may adhere to the plenum chamber 3200 (for instance, to the first flange region of flange 3105) when the first assembly component 6002 engages with the second assembly component 6004, for example when the assembly components 6002, 6004 are brought together. In other forms, the plenum chamber 3200 may comprise an adhesive surface on a patient-facing surface of the plenum chamber 3200, for example the plenum chamber may comprise one or more adhesive layers mounted to a patient-facing surface of the plenum chamber 3200a, in order to adhere the plenum chamber 3200 to the seal-forming structure 3100.

Exemplary forms of patient interface 3000 that may be assembled by use of the assembly apparatus 6000 may be any of the forms of technology described earlier. In particular, the assembly apparatus 6000 may be suited for use in the assembly of a patient interface 3000, such as shown by way of example in FIGS. 3 to 12B, that comprises at least one adhesive layer 3190 on a patient-facing side of the seal-forming structure 3100 for adhering the assembled patient interface 3000 to the patient's face in use and two portions that are adhered together to assemble the patient interface 3000. For example, the adhesive layer 3190 may be applied to the rest of the patient interface 3000 by adhering the adhesive layer 3190 to another part of the patient interface 3000, for example the seal-forming structure 3100 and/or the plenum chamber 3200. In examples, the adhesive layer 3190 may have an adhesive surface on both sides, i.e. the patient-facing side and the non-patient-facing side. (It should be appreciated that the patient-facing side and the non-patient-facing side will not always be facing and not facing the patient respectively, depending on where the adhesive layer is positioned at any given moment. However, these labels are used for convenience to identify the respective sides and to refer to their orientation when the patient interface 3000 is being worn by a patient). In some forms, prior to the assembly process, a patient-facing removable layer 3120 may cover the patient-facing side of the adhesive layer 3190 and a non-patient-facing removable layer 3180 may cover the non-patient-facing side of the adhesive layer 3190.

In certain forms, for example as illustrated, the first assembly component 6002 may comprise a first region 6100 and the second assembly component 6004 may comprise a second region 6200. The respective assembly components are configured so that, in use, the first region 6100 may be positioned to face the second region 6200 when the first assembly component 6002 engages with the second assembly component 6004.

### 4.4.2 Receiving Regions

The first region 6100 may further comprise a first receiving region 6110 configured to receive a first portion of the patient interface 3000 in use. That is, the first receiving region 6110 may have a structure that allows the first portion of the patient interface 3000, for example the plenum chamber 3200 or a sub-assembly comprising the plenum chamber 3200 and at least part of the seal-forming structure 3100, to be conveniently placed in close proximity to the first receiving region 6110 and/or retained by the region 6110. In certain forms, the first receiving region 6110 may comprise a substantially continuous surface against which a part, for example a substantial part, of a non-patient-facing surface of the seal-forming structure 3100 of the patient interface 3000 substantially abuts when the first portion is received by the first receiving region 6110. The part of the seal-forming structure 3100 abutting the first receiving region 6110 may, in some forms, be a first flange region integrally connected to the plenum chamber 3200. A substantially continuous surface may help to ensure application of force across a majority of the surface of the first portion when the second portion is caused to adhere to the first portion, as described later.

Additionally or alternatively, the first receiving region 6110 may have a shape that substantially corresponds to a natural shape of the non-patient-facing side of the seal-forming structure 3100. The “natural shape” may be considered to be the shape of the component in the absence of a distorting force. For example, to substantially correspond to the shape of certain examples of the seal-forming structure 3100 shown in FIGS. 6 to 10 in which the seal-forming structure 3100 has a non-patient-facing side that is substantially concave, the first receiving region 6110, as shown in FIGS. 16, 25, 36, 41 and 44, may be substantially convex when viewed in cross-section from a side direction. This side direction may correspond to a direction across the seal-forming structure 3100 in the medial-lateral direction relative to the patient when the patient interface 3000 is being worn. The shape of the convexity of the first receiving region 6110 may be configured to correspond to the shape of the concavity on the non-patient-facing side of the seal-forming structure 3100 (e.g. the same shape but inverted) so that the seal-forming structure 3100 can be positioned in close abutment with the first receiving region 6110. In certain forms, the first receiving region 6110 may also comprise one or more concave regions 6112, which may be positioned to correspond to convex regions on the non-patient-facing side of the seal-forming structure 3100. In certain forms, the concave regions 6112 may be positioned at opposing top and bottom regions of the first receiving region 6110, where “top” and “bottom” in this context correspond to regions that, when the seal-forming structure 3100 is received by the first receiving region 6110, are immediately adjacent regions of the seal-forming structure 3100 that, when placed on the patient's face, will be positioned in a superior medial position and an inferior medial position respectively on the patient's face. The first receiving region 6110 may further comprise a cavity 6124 to accommodate the plenum chamber 3200 in use, so that plenum chamber 3200 remains substantially undeformed when the first assembly component 6002 engages with the second assembly component 6004. The cavity 6124 may be located in a central region of the first receiving region 6110 and have a shape that is complementary to the shape of the plenum chamber 3200. In some forms, the cavity 6124 may be a through-hole in the first receiving region 6110 while in other forms the cavity 6124 may be a blind hole in the first receiving region 6110.

Turning now to the second assembly component 6004, the second region 6200 may further comprise a second receiving region 6210 configured to receive a second portion of the patient interface 3000 in use, for example an adhesive layer 3190 and/or part of the seal-forming structure 3100. That is, the second receiving region 6210 has a structure that allows the second portion to be conveniently placed in close proximity to the second receiving region 6210 and/or retained by the region.

In certain forms, the second receiving region 6210 may comprise a substantially continuous surface against which a substantial part of the second portion, for example a non-patient-facing surface of the seal-forming structure 3100, substantially abuts when the first and second portions are adhered together in use. A substantially continuous surface may help to ensure application of force across a majority of the surface of the second portion when the second portion is caused to adhere to the first portion, as described later.

Additionally or alternatively, the shape of the second receiving region 6210 may be substantially complementary to the shape of the first receiving region 6110, e.g. the two regions may have the same but inverted contours to each other. This may allow the first and second receiving regions to be placed in a meshed configuration with respect to each other, i.e. a configuration in which the respective surfaces are in close abutment.

In some forms, as a result of the complementary shapes, the second receiving region 6210 may have a shape that substantially corresponds to a natural shape of a patient-facing side of the seal-forming structure 3100. In some forms, the shape of the non-patient-facing side of the seal-forming structure 3100 is the same as, or closely similar to, the patient-facing side of the seal-forming structure, but inverted. It is noted that this shape may or may not be complementary to a natural shape of the adhesive layer 3190. In some forms, the adhesive layer 3190 may have a planar natural shape and it may be distorted into a shape to match the shape of the seal-forming structure 3100 when the adhesive layer 3190 is pushed into contact with the seal-forming structure 3100, as described below. In other forms, the adhesive layer 3190 may have a natural shape that matches the shape of the seal-forming structure 3100.

For example, to substantially correspond to the shape of certain examples of seal-forming structure 3100 shown in FIGS. 6 to 10 in which the seal-forming structure 3100 has a patient-facing side that is substantially convex, the second receiving region 6210, as shown in FIGS. 16, 29, 36 and 41, may be substantially concave when viewed in cross-section from the same side direction as described earlier. In certain forms, the second receiving region 6210 may also comprise one or more convex regions 6114, which may be positioned to correspond to concave regions on the patient-facing side of the seal-forming structure 3100. These convex regions may also be positioned to correspond to the concave regions 6112 of the first receiving region 6110, described above.

The first and second assembly components are configured so that, when the first and second receiving regions are engaged the desired alignment between the first and second portions of the patient interface 3000 is achieved. This helps to reliably and repeatedly adhere the first and second portions together in the intended position.

### 4.4.3 Retaining Structures

In some forms of the technology, the first assembly component 6002 may further comprise a first retaining structure 6120 to maintain the first portion of the patient interface 3000 in a substantially fixed position in use with respect to the first assembly component 6002 during assembly of the patient interface 3000. A variety of retaining mechanisms may be used in different forms and, in some forms, the first retaining structure 6120 may comprise a plurality of retaining mechanisms. In some forms, the first retaining structure 6120 may hinder or prevent sideways movement of the first portion of the patient interface 3000 when it is received by the first receiving region 6110 (i.e. movement substantially parallel to the surface of the first receiving region 6110). The first retaining structure 6120 may or may not prevent the first portion of the patient interface 3000 from moving into or out of the first receiving region 6110 (i.e. in a direction perpendicular to the surface of the first receiving region 6110). In the illustrated forms, the first retaining structure 6120 may comprise the cavity 6124 which is configured to receive at least a portion of the plenum chamber 3200. The size and shape of the cavity 6124 may be configured to prevent or restrict sideways movement of the plenum chamber 3200 when it is received in the cavity.

Additionally, or alternatively, for example in the case of the forms of FIGS. 13 to 35, the first retaining structure 6120 may comprise one or more wall sections positioned so as to be around the perimeter of the seal-forming structure 3100 when the seal-forming structure 3100 is received in the first receiving region 6110. In some forms, the first receiving region 6110 may comprise a recess 6122 within which the seal-forming structure 3100 is received and the walls around the recess 6122 may act to substantially prevent or restrict sideways movement of the seal-forming structure 3100.

In other forms, for example in the case of the forms of FIGS. 36 to 44, the first retaining structure 6120 may comprise one or more wall sections positioned so as to be around the perimeter of a first flange region of flange 3105 when the plenum chamber 3200 is received in the first receiving region 6110. In some forms, the first receiving region 6110 may comprise a recess 6122 within which the flange 3105 is received and the walls around the recess 6122 may act to substantially prevent or restrict sideways movement of the plenum chamber 3200 in use.

In still other forms of the technology, the first retaining structure 6120 may comprise one or more clips for engaging with the first portion of the patient interface 3000.

In some forms of the technology, the second assembly component 6004 may further comprise a second retaining structure 6220 to maintain the second portion of the patient interface 3000, for example the adhesive layer 3190 and/or the seal-forming structure 3100, in a substantially fixed position in use with respect to the second assembly component 6004 during assembly of the patient interface 3000. In certain forms, the second retaining structure 6220 may comprise one or more slots to receive respective parts of the second portion, for example adhesive layer 3190, a removable layer connected to the adhesive layer 3190, a seal-forming structure 3100 or a removable layer 3120 connected to the seal-forming structure 3100. For example, in the exemplary forms of FIGS. 25 to 35 and 36 to 41, the second assembly component 6004 comprises a first slot 6222 which is configured to receive a first tab 3194 of a patient-facing removable layer 3120 connected to the adhesive layer 3190 (as shown in FIG. 12B). The second retaining structure 6220 may further comprise a second slot 6224 which is configured to receive a second tab 3196 of a patient-facing removable layer 3120 connected to the adhesive layer 3190 (also shown in FIG. 12B). The first and second slots 6222 and 6224 may be positioned substantially opposite each other, i.e. so that the adhesive layer 3190 may be positioned between the slots. This arrangement may enable particular stability in retaining the adhesive layer 3190 in position during use.

In certain forms, one or both of the first assembly component 6002 and the second assembly component 6004 may comprise part of a fastening mechanism to hold them in a fixed position relative to each other. For example, the first assembly component 6002 may further comprise a first fastening member 6130 and the second assembly component 6004 may further comprise a second fastening member 6230. The first assembly component 6002 and the second assembly component 6004 may be fastened together when they engage with each other, for example when the first receiving region 6110 and the second receiving region 6210 are in a meshed configuration. Alternatively, they may be fastened together when the first receiving region 6110 and the second receiving region 6210 are engaged but in a configuration in which there is a gap separating the first receiving region 6110 and the second receiving region 6210, such as described further below.

### 4.4.4 Method of Assembly

There will now be described how an assembly apparatus 6000 may be used in the assembly of a patient interface 3000 in certain forms of the technology.

Initially, the first and second portions of the patient interface 3000 may be separate to each other. As shown by way of example in FIG. 12B, the adhesive layer 3190 may be provided on one or more backing layers. For example, a patient-facing removable layer 3120 and/or a non-patient-facing removable layer 3180 may cover the adhesive surfaces of the adhesive layer 3120.

The first portion of the patient interface 3000 may be received by the first receiving region 6110 of the first assembly component 6002. For example, as in the case of the forms of the technology shown in FIGS. 13 to 47, the first assembly component 6002 may be positioned with the first receiving region 6110 facing upwards and the first portion of the patient interface 3000 may be placed on the first receiving region 6110. The first portion of the patient interface 3000 may also be retained by the first retaining structure 6120.

Also, the second portion of the patient interface 3000 may be received by the second receiving region 6210 of the second assembly component 6004. For example, as in the case of the forms of the technology shown in FIGS. 13 to 47, the second assembly component 6004 may be positioned with the second receiving region 6210 facing substantially upwards and the second portion may be placed on the second receiving region 6210. The second portion may also be retained by the second retaining structure 6220. If there is a non-patient-facing removable layer 3180 on a non-patient-facing side of the second portion, that layer may be removed.

The first and second assembly components 6002 and 6004 may then be engaged. For example, the two components may be brought together. In some forms, the bringing of these two components together may comprise direct physical engagement between parts of the components, for example interlocking of connecting portions. In other forms, the bringing of these two components together may comprise bringing two portions of the components into close proximity, for example substantial abutment. In engaging the first and second assembly components 6002 and 6004, the first and second receiving regions 6110 and 6210 are positioned adjacent to each other. In some forms, when in the engaged configuration, the first and second receiving regions 6110 and 6210 may be in a meshed configuration. As will be described in more detail below, in some exemplary forms, the first and second receiving regions 6110 and 6210 may face each other with a gap separating them. This configuration may be a stable one, i.e. the assembly apparatus 6000 may be configured so that the first and second assembly components 6002 and 6004 may remain in the relative position in which this gap is present without a retaining force being applied to the assembly apparatus 6000.

The engagement of the first and second receiving regions 6110 and 6210 may result in the second portion being brought into contact with the first portion of the patient interface 3000. In some forms, this contact configuration may occur after urging the first and second assembly components 6002 and 6004 together. In the contact configuration, at least parts of the first and second portions, for example the seal-forming structure 3100 and the adhesive layer 3190, lie between the first receiving region 6110 and the second receiving region 6210. Contact between the adhesive surface(s) of the first and second portions, for example between the non-patient-facing surface of the adhesive layer 3190 and the patient-facing side of the seal-forming structure 3100, or between the adhesive layer 3190a or 3122 and the first flange region of the flange 3105, may cause the first and second portions to adhere together.

It may be beneficial or necessary for a user to apply additional force to urge the two assembly components together in order to achieve and/or enhance the adhesion.

After forming the assembled patient interface 3000 by adhering the first and second portions together, the first assembly component 6002 may be separated from the second assembly component 6004. The assembled patient interface 3000, as shown in FIG. 35, may remain attached to the second assembly component 6004 due to the second retaining structure 6220, for example the first tab 3194 and the second tab 3196, continuing to secure the seal-forming structure 3100, or the adhesive layer 3190 or the removable layer attached thereto, and consequently the rest of the assembled patient interface which is adhered to the seal-forming structure 3100 and/or the adhesive layer 3190, to the second assembly component 6004. The patient 1000 may then disengage the assembled patient interface 3000 from the second retaining structure 6220, for example by slipping the first tab 3194 and the second tab 3196 out of the first slot 6222 and the second slot 6224 respectively. In other forms, for example in the absence of a second retaining structure 6220 when the first assembly component 6002 is separated from the second assembly component 6004, the assembled patient interface 3000 may be left sitting on the first assembly component 6002.

The patient interface 3000 may subsequently be mounted to a patient's face by adhering the patient-facing side of the seal-forming structure 3100 to the patient's face. If a patient-facing removable layer 3120 covers the patient-facing side of the adhesive layer 3190 or 3190b, this is removed to expose an adhesive surface of the adhesive layer for adhesion to the patient's face.

### 4.4.5 Further Description of Exemplary Forms of Assembly Apparatus

FIGS. 13 to 24 illustrate a first exemplary form of the assembly apparatus 6000. FIGS. 25 to 35 illustrate a second exemplary form of the assembly apparatus 6000. FIGS. 36 to 40 illustrate a third exemplary form of the assembly apparatus 6000. FIGS. 41 to 47 illustrate further exemplary forms of the assembly apparatus 6000. Many of the features and function of these forms have already been described. Further details of the arrangement, configuration and working of the assembly apparatus 6000 in the first, second and third forms are given below.

### 4.4.5.1 First Exemplary Form of the Assembly Apparatus

In the first exemplary form, the assembly apparatus 6000 comprises a two-part first assembly component 6002 in which the first assembly component 6002 comprises an inner member 6008 and an outer member 6006. The inner and outer members may be configured to move relative to each other and, in doing so, may bring the first receiving region 6110 into a position adjacent to the second receiving region 6210 to apply the adhesive layer 3190 to the seal-forming structure 3100.

In exemplary forms, such as illustrated, the outer member 6006 may take the form of a sleeve positioned around the outer perimeter of the inner member 6008. The sleeve may be open at an upper end so that the upper surface of the inner member 6008 is not covered by the outer member 6006. The first region 6100 of the first assembly component 6002 may be located on the upper surface of the inner member 6008 as shown in the Figs. The first region 6100 includes the first receiving region 6110 and the inner member 6008 may therefore comprise the first receiving region 6110. The first retaining structure 6120, in this form of the assembly apparatus 6000, comprises a recess 6122 in addition to the cavity 6124. The seal-forming structure 3100 is configured to substantially lie within the recess 6122. Therefore, the first receiving region 6110 may be considered to be the surface of the inner member 6008 substantially within the recess 6122.

The inner member 6008 and the outer member 6006 may move with respect to each with a sliding motion, for example along the axis of the sleeve in the case of the outer member 6006 being in the form of a sleeve. In moving relative to each other, the upper surface of the inner member 6008 may remain uncovered by the outer member 6006. To achieve this, the outer member 6006 may be configured to fit snugly around the inner member 6008 to permit relative movement of the two components. In some forms, one of the inner or outer member may comprise one or more grooves and the other of the inner or outer member may comprise one or more projections, the projections and grooves being configured to allow the projections to engage with the grooves and to slide along them.

In a stable configuration of the first assembly component 6002, i.e. a configuration in which it is able to remain without the application of force, the upper surface of the inner member 6008 may be recessed from the upper end of the outer member 6006.

The first assembly component 6002 may comprise an elastic member 6010 configured to return the outer member 6006 and the inner member 6008 to an original configuration. In some forms, the original configuration may be the stable configuration described above. The elastic member 6010 may take the form of any one or more resilient members configured to act between the outer member 6006 and the inner member 6008. As shown in FIGS. 14, 17, 18 to 20, and 21 to 24, in the illustrated form, the elastic member 6010 is an elastic band with one portion attached to the inner member 6008 and another portion looped around a guiding member 6300 of the outer member 6006. The point at which the elastic member 6010 is attached to the inner member 6008 may be substantially at the same height from a bottom edge 6502 of the inner member 6008 as the height of the guiding member 6300 from the bottom edge 6502 of the inner member 6008. This may ensure that the elastic member 6010 has the least tension when the inner member 6008 and the outer member 6006 are in the original, stable configuration with the upper surface of the inner member recessed from the upper end of the outer member. When the outer member 6006 is pushed towards the bottom edge 6502 of the inner member 6008, the elastic member 6010 stretches because the guiding member 6300 moves away from the point where the elastic member 6010 is attached to on the inner member 6008. The stretched elastic member 6010 has a tension which causes the outer member 6006 to spring back to the initial position. In some forms the first assembly component 6002 may be configured so that the elastic member 6010 is under tension when the inner and outer members are in the original, stable configuration. This may ensure that, when no force is applied to move the inner and outer members relative to each other, they move fully back to their original configuration. The inner and outer members 6006 and 6008 may comprise one or more stops to prevent the movement of the inner member relative to the outer member beyond this original configuration.

In this exemplary form of the technology, the first assembly component 6002 is hingedly attached to the second assembly component 6004, i.e. the assembly apparatus 6000 may include a hinge 6030 which connects the second assembly component 6004 to the first assembly component 6002. More specifically, the second assembly component 6004 may be connected via the hinge to the outer member 6006. In a closed configuration, the second assembly component 6004 may abut against the upper end of the outer member 6006 and may substantially cover the upper end of the sleeve-form of the outer member 6006. Therefore, in this form, the second assembly component 6004, may be considered to be a lid to the first assembly component 6002.

The assembly apparatus 6000 may comprise a fastening mechanism to hold the second assembly component 6004 in the closed configuration. In the illustrated form, the second assembly component 6004 comprises the second fastening member 6230 which may be secured to the first fastening member 6130 provided to the outer member 6006 in the closed configuration.

FIGS. 16 to 18 illustrate a configuration of the assembly apparatus 6000 in which the second assembly component 6004 is in an open position with respect to the outer member 6006 (i.e. the lid is open), thereby providing access to the first receiving region 6110 and the second receiving region 6210. A first portion of the patient interface 3000 may be provided to the first receiving region 6110 of the inner member 6008. In one example, the first portion may be in the form of a sub-assembly comprising the plenum chamber 3200 and the seal-forming structure 3100, such as shown in FIGS. 6, 8 and 9, and the seal-forming structure 3100 may be located substantially within the recess 6122 and the plenum chamber 3200 may be substantially located within the cavity 6124. That is, the recess 6122 may be sufficiently large to receive all of the seal-forming structure 3100. In forms in which there is no recess 6122, the first receiving region 6110 may be sufficiently large to receive all of the seal-forming structure 3100. Also, the second portion may be in the form of adhesive layer 3190 may be provided to the second assembly component 6004, for example by inserting the first tab 3194 through the first slot 6222 and the second tab 3196 through the second slot 6224. In another example, the first portion may be in the form of a plenum chamber 3200 comprising a first flange region, such as shown in FIGS. 10 and 12A and the plenum chamber 3200 may be substantially located within the cavity 6124. In this example, the second portion may be in the form of a seal-forming structure 3100 that comprises a plurality of adhesive layers 3190a and 3190b.

FIGS. 19 to 21 illustrate a closed configuration of the assembly apparatus 6000. In this configuration, the second assembly component 6004 and the outer member 6006 are closed together by rotating the second assembly component 6004 around the hinge 6030 towards the outer member 6006 (i.e. the lid is closed). Further, the second assembly component 6004 is secured in the closed position with respect to the outer member 6006 by engaging the first fastening member 6130 and the second fastening member 6230 as mentioned previously.

In this closed configuration, and when the inner and outer members are in their original, stable configuration, the first and second receiving regions 6110 and 6210 are positioned facing each other with a gap separating them. This gap is due to the recess of the upper surface of the inner member 6008 relative to the upper end of the outer member 6006. This means that the patient-facing side (the second surface) of the seal-forming structure 3100 and the non-patient-facing side 3126 of the adhesive layer 3190 face each other in this configuration with a gap between the seal-forming structure 3100 and the adhesive layer 3190.

FIGS. 22 to 24 illustrate an assembly configuration of the assembly apparatus 6000. In this configuration, the outer member 6006, together with the second assembly component 6004 which sits on the upper end of the outer member 6006, is pushed downwards. This causes the second assembly component 6004 and the outer member 6006 to move downwardly relative to the inner member 6008 and causes the second receiving region 6210 to move into engagement with the first receiving region 6110. This eliminates the gap between the seal-forming structure 3100 and the non-patient-facing side 3126 of the adhesive layer 3190 and causes the adhesive layer to be affixed to the seal-forming structure 3100 of the patient interface 3000, thereby forming the assembled patient interface 3000, as described above. When the downward force is released from the top of the second assembly component 6000, the elastic member 6010 causes the assembly apparatus 6000 to return to its stable configuration. The lid can then be opened and the assembled patient interface 3000 can be retrieved, ready for use.

### 4.4.5.2 Second Exemplary Form of the Assembly Apparatus

In the second exemplary form of the assembly apparatus 6000, shown in FIGS. 25 to 35, the first assembly component 6002 and the second assembly component 6004 may be separate components. In use, they are able to be brought together to engage and thereby apply the adhesive layer 3190 to the seal-forming structure 3100 of the patient interface 3000.

In certain forms, the first region 6100 of the first assembly component 6002 comprises the first receiving region 6110. The shape of the first receiving region 6110 may be as described earlier and may include the cavity 6124 for receiving the first portion, for example at least a portion of the plenum chamber 3200 in use. In use, the second portion, for example the seal-forming structure 3100, is configured to be placed on the first receiving region 6110, as shown in FIG. 27.

The first assembly component 6002 may also comprise first fastening members 6130 configured to enable the first and second assembly components to be fastened together. The first fastening members 6130 may comprise one or more male or one or more female members, each configured to mate with a respective member on the second assembly component 6004. In the illustrated example, the first fastening members 6130 comprise two protrusions on diagonally distal ends of the first region 6100.

In certain forms, the second region 6200 of the second assembly component 6004 comprises the second receiving region 6210. As has already been described, the second receiving region 6210 of the second assembly component 6004 may have a shape that substantially complements the shape of the first receiving region 6110 such that the two regions are able to be brought together into engagement, for example in a meshed configuration with the seal-forming structure 3100 and the adhesive layer 3190 being sandwiched between the first receiving region 6110 and the second receiving region 6210. This allows maintenance of the shape of the seal-forming structure 3100 when a squeezing force is applied to the seal-forming structure 3100 in order to adhere the adhesive layer 3190 to it. As the plenum chamber 3200 is received in the cavity 6124, the shape of the plenum chamber 3200 may also remain unaltered when the first assembly component 6002 and the second assembly component 6004 engage together to form the assembled patient interface 3000.

The second assembly component 6004 may further comprise second fastening members 6230 configured to enable the first and second assembly components to be fastened together. The second fastening members 6230 may comprise one or more male or one or more female members, each configured to mate with a respective member on the first assembly component 6002. In the illustrated example, for example as seen in FIG. 28, the second fastening members 6230 comprise two holes on diagonally distal ends of the second assembly component 6004. The second fastening members 6230 are configured to receive the first fastening members 6130 in use to maintain engagement between the first assembly component 6002 and the second assembly component 6004. The fastening members 6130 and 6230 may also facilitate alignment between the first and second assembly components.

As illustrated in FIG. 30, in use, the second assembly component 6004 receives the second portion, for example the adhesive layer 3190, or the patient-facing removable layer attached to the adhesive layer 3190, may be retained by the second retaining structure 6220, for example the first tab 3194 and second tab 3196 may be respectively inserted into the first slot 6222 and the second slot 6224 of the second assembly component 6004. The non-patient-facing side 3126 of the adhesive layer 3190 is configured to face away from the second region 6200 such that, when the first receiving region 6110 and the second receiving region 6210 are arranged to face each other, the patient-facing side (second surface) of the seal-forming structure 3100 faces the side 3126.

When the first fastening members 6130 are received by the second fastening members 6230 and the first assembly component 6002 is pressed against the second assembly component 6004, the first assembly component 6002 engages with the second assembly component 6004. This causes the first and second portions to be adhered together, thereby forming the assembled patient interface 3000.

The second assembly component 6004 may comprise one or more snap-fit members 6232 which may be configured to be attached to a portion (such as a notch) of the first assembly component 6002 to maintain engagement between the first assembly component 6002 and the second assembly component 6004. The snap-fit members may be located on a side edge 6242 of the second assembly component 6004. The snap-fit members 6232 may be configured to attach to corresponding members on the first assembly component 6002, which may be provided in a corresponding position on a side edge 6142 thereof.

### 4.4.5.3 Third Exemplary Form of the Assembly Apparatus

In the third exemplary form, the assembly apparatus 6000 comprises a first assembly component 6002, a second assembly component 6004 and one or more elastic members 6010. The first assembly component 6002 may comprise an inner member 6008 and an outer member 6006. The first assembly component 6002 may be hingedly attached to the second assembly component 6004. For example, as in the illustrated form of the technology, the outer member 6006 of the first assembly component 6002 may be hingedly attached to the second assembly component 6004. For example, a hinge 6030 which attaches the outer member 6006 and the second assembly component 6004 may be a living hinge and consequently, the outer member 6006 and the second assembly component 6004 may be formed together as a single component, as shown in FIG. 39. In other forms, the outer member 6006 of the first assembly component 6002 and the second assembly component 6004 may be separate components that are joined by a hinge.

The second assembly component 6004 may be rotated around the hinge towards the first assembly component 6002 to change the assembly apparatus 6000 from an open configuration to a closed configuration. As in the first exemplary form, the second assembly component 6004 may be considered to be a lid to the first assembly component 6002.

The first assembly component 6002 may comprise one or more first alignment structures 6232A, 6233A, 6234A which may engage with one or more second alignment structures 6232, 6233, 6234 that are comprised as part of the second assembly component 6004 to align the second assembly component 6004 with the first assembly component 6002 in the closed configuration. In the illustrated form of FIGS. 36-40, the first alignment structures each comprise one or more protrusions and the second alignment structures comprise one or more openings, each configured to receive one of the protrusions of the first alignment structures. The alignment structures may engage with each other through snap-fit, friction fit or other appropriate means of engagement. In alternative forms of the technology, the second alignment structures may each comprise one or more protrusions and the first alignment structures may comprise one or more openings, each configured to receive one of the protrusions of the second alignment structures. The alignment structures may act, in use, to facilitate alignment between the first and second assembly components, which helps to ensure the first and second portions of the patient interface 3000 are adhered together in the desired manner. The alignment structures may also, or alternatively, act to fasten the first assembly component 6002 to the second assembly component 6004 (and may consequently alternatively be referred to as fastening structures).

The inner 6008 and outer 6006 members of the first assembly component 6002 may be configured to move relative to each other. In the form of the technology illustrated in FIGS. 36 to 40, the inner member 6008 comprises a column 6038, which may extend upwardly from a base 6502. The outer member 6006 may comprise an orifice 6036, and the orifice 6036 may be configured to receive at least a portion of the column 6038, i.e. the column 6038 may fit within the orifice 6036. The inner member 6008 and the outer member 6006 may move with respect to each, for example in a sliding motion along a longitudinal axis of the orifice 6036 in the case of the outer member 6006 comprising the orifice 6036 (which direction may also correspond to a longitudinal axis of the column 6038). In moving relative to each other, the upper surface of the inner member 6008 may remain uncovered by the outer member 6006. To achieve this, the orifice 6036 of the outer member 6006 may be configured to fit snugly around the column 6038 of the inner member 6008.

In some forms, one of the inner or outer member may comprise one or more grooves and the other of the inner or outer member may comprise one or more projections, the projections and grooves being configured to mutually engage and to permit sliding movement of the inner member 6008 relative to the outer member 6006. In the illustrated form of the technology, the outer member 6006 may comprise a wall 6310 which may extend substantially downwards from an edge of the orifice 6036 in a direction that is away from the upper surface of the outer member, and towards the inner member 6008 when the members are assembled. The wall 6310 may extend around a some or all of the circumference of the orifice 6036. The wall 6310 may comprise one or more first guiding members 6304 on an inner-facing surface. The column 6038 may comprise one or more second guiding members 6306 on a side outer-facing wall. Each of the one or more first guiding members 6304 may be configured to engage with each of the one or more second guiding members 6306 to facilitate sliding movement of the inner member 6008 relative to slide substantially the outer member 6006 in use. In the illustrated form of the technology, there are four first guiding members 6304 which may engage with four second guiding members 6306 in use. Alternatively, there may be other numbers of first guiding members 6304 engaging with a corresponding number of second guiding members 6306.

The first assembly component 6002 may comprise a first region 6100 which may further comprise a first receiving region 6110 and/or a first retaining structure 6120. The first receiving region 6110 may, at least in part, be substantially formed on an upper surface of the inner member 6008. For example, the first receiving region 6110 may comprise the upper surface of the column 6038. The first receiving region 6110 may comprise a recess 6122 and/or a cavity 6124. In the exemplary form of the technology illustrated by FIGS. 36 to 40, the upper surface of the column 6038 forms the cavity 6124.

In some forms, the first region 6100 may further comprise an upper surface of the outer member 6006. In a stable configuration of the inner and outer members, the upper surface of the column 6038 of the inner member 6008 may be recessed from the upper surface of the outer member 6006, forming a recess 6122 such has previously been described, with the edge of the recess 6122 being formed by an upper portion of the wall 6310.

The inner and outer members may be configured so that the recess 6122 may receive at least a portion of the flange 3105 in use. The cavity 6124 may be configured to receive at least a portion of the plenum chamber 3200 in use. For instance, when the plenum chamber 3200 comprising the flange 3105 is positioned in the first receiving region 6110, the flange 3105 may be substantially flush with the rest of the first region 6100 (which may be substantially provided by the outer member 6006). The advantage of this configuration is that the flange 3105 and/or the plenum chamber 3200 may avoid being subject to deformative forces when the first assembly component 6002 and the second assembly component 6004 engage together to form the assembled patient interface 3000.

Alternatively, the upper surface of the column 6038 of the inner member 6008 may be configured to substantially align with the upper surface of the outer member 6006, in use, in a stable (open or closed) configuration, to form the first region 6100 which comprises the first receiving region 6110 region and the first retaining region 6120.

The first region 6100 may further comprise indicators 6126 to indicate where the plenum chamber 3200 is to be located in use. In the illustrated example, the indicators 6126 are located on an upper surface of the outer member 6006 on either side of the first receiving region 6110.

The second assembly component 6004 may comprise a second region 6200 which may further comprise a second receiving region 6210 and/or a first slot 6222 and a second slot 6224. The seal-forming structure 3100 comprising the adhesive layers 3190b, 3190a (or 3122) may be received by the second receiving region 6210 in use. The removable layer 3120 (configured to cover the patient-facing side of the adhesive layer 3190a) of the seal-forming structure 3100 may comprise a first tab 3194 and a second tab 3196 which may be received by the first slot 6222 and the second slot 6224 respectively in use. Indicators 6226 and 6228 may indicate to a user where the tabs are to be inserted during use. For example, the indicators 6226 and 6228 may be located on a surface of the second region 6200.

The orientation of the seal-forming structure 3100 when received by the second receiving region 6210 may be such that the adhesive layer 3190a faces the first region 6100 and the patient-facing removable layer 3120 faces the second region 6200 in use. If the adhesive layer 3190a (or 3122) is protected by a non-patient facing removable layer 3180, this layer may be removed to expose the adhesive layer 3190a (or 3122) before or after the seal-forming structure 3100 is received by the second receiving region 6210.

The inner 6008 and outer 6006 members may be configured to move relative to each other from a closed stable configuration to an assembly configuration and, in doing so, may bring the first receiving region 6110 into a position adjacent to the second receiving region 6210 to adhere the seal-forming structure 3100 to the plenum chamber 3200.

The first assembly component 6002 may comprise one or more elastic members 6010 configured to return the outer member 6006 and the inner member 6008 to an original configuration. In some forms, the original configuration may be the stable configuration described above. The one or more elastic members 6010 may take the form of any one or more resilient members configured to act between the outer member 6006 and the inner member 6008. As shown in FIGS. 38 and 40, in this illustrated form, the one or more elastic members 6010 comprise two springs 6010. Each spring 6010, as shown in FIG. 40, may extend upwardly from base 6502 and may be located on either side of the column 6038. A lower portion of each spring 6010 may be attached to base 6502 of the inner member 6008 or each spring 6010 may be integrally formed with base 6502, for example through a moulding process. In other forms, the spring 6010 may act on the base 6502 without being directly connected to it. An upper portion of the spring 6010 may be configured to act against a respective bearing surface 6302 of the outer member 6006. Each of the bearing surfaces 6302, as shown in FIG. 39, may be located on an underside of the outer member 6006, and may be located on either side of the orifice 6036 and/or the wall 6310.

The elastic member(s) 6010 may be configured to be relatively non-compressed when the inner member 6008 and the outer member 6006 are in the original, stable configuration. When the outer member 6006 is pushed downardly, i.e. towards a bottom edge 6502 of the inner member 6008, the elastic member(s) 6010 may be configured to compress under the pushing force. In the illustrated form of the technology, the upper surface of the second assembly component 6004 comprises indicators 6236 to indicate to a user to press down or push down on the second assembly component 6004 such that, in a closed configuration, the force is transferred through the second assembly component 6004 and the outer member 6006 to the elastic member(s) 6010 which is/are then compressed.

The compressed elastic member(s) 6010 has/have stored force which causes the outer member 6006 to spring back to the initial position once the downwards force is removed. The inner and outer members 6006 and 6008 may comprise one or more stops to prevent the movement of the inner member relative to the outer member beyond this original configuration. For instance, each of the first guiding members 6304 may include a stop which may be configured to engage with a corresponding stop of each of the second guiding members 6306 to ensure that the sliding motion between the inner member 6008 and the outer member 6006 does not extend beyond their relative positions in the original or stable configuration.

In the open position, the second assembly component 6004 provides access to the first receiving region 6110 and the second receiving region 6210. The plenum chamber 3200 may be provided to the first receiving region 6110 and the seal-forming structure 3100 may be provided to the second receiving region 6210. In the closed configuration of the assembly apparatus 6000, the second assembly component 6004 and the outer member 6006 are closed together by rotating the second assembly component 6004 around the hinge 6030 towards the outer member 6006 (i.e. the lid is closed). Further, the second assembly component 6004 may be aligned and/or secured in the closed position with respect to the outer member 6006 by engaging each of the one or more first alignment members 6232, 6233, 6234 with each of the respective one or more second alignment members 6232A, 6233A, 6234A as mentioned previously.

In this closed configuration, and when the inner and outer members are in their original, stable configuration, the first and second receiving regions 6110 and 6210 may be positioned facing each other with a gap separating them. This means that the patient-facing side of the first flange region and the non-patient-facing side of the seal-forming structure 3100 comprising the adhesive layer 3190a (Or 3122) face each other in this configuration with a gap between the first flange region and the adhesive layer 3190a (or 3122) of the seal-forming structure 3100.

In the assembly configuration, the outer member 6006, together with the second assembly component 6004 which sits on the upper end of the outer member 6006, is pushed downwards. This causes the second assembly component 6004 and the outer member 6006 to move downwardly relative to the inner member 6008 and causes the second receiving region 6210 to move into engagement with the first receiving region 6110. This eliminates the gap between the first flange region and the adhesive layer 3190a (or 3122) of the seal-forming structure 3100 and causes the plenum chamber 3200 to be adhered to the seal-forming structure 3100, thereby forming the assembled patient interface 3000. When the downward force is released from the top of the second assembly component 6000, the elastic member(s) 6010 cause(s) the assembly apparatus 6000 to return to its stable configuration. The lid or the second assembly component 6004 can then be opened and the assembled patient interface 3000 can be retrieved, ready for use.

### 4.4.6 Two-Part Second Assembly Component

In certain forms of the technology, for example as shown in FIGS. 41-44, the second assembly component 6004 may be formed in two parts. That is, the second assembly component 6004 may comprise a first assembly part 6062 and a second assembly part 6064. The first assembly part 6062 may comprise the second receiving region 6210.

The first assembly part 6062 and the second assembly part 6064 may, in some forms, be able to be moved relative to each other to adopt certain configurations. In certain forms, in one of these configurations, the first assembly part 6062 and the second assembly part 6064 may be connected together and they may together form a cavity. The cavity may be suitable for storing certain components, for example one or more portions of the patient interface 3000. In certain forms, the assembly parts may be configured such that the cavity may be suitable for storing seal-forming structures 3100, such as shown in FIG. 43. Since the patient may use a new seal-forming structure 3100 regularly, keeping them in a storage cavity in the assembly apparatus 6000 may be convenient.

As shown in FIGS. 41-44, in examples, the first assembly part 6062 may be a curved panel in shape, with a central part of the first assembly part 6062 comprising the second receiving region 6210 on one side thereof. The shape of the second receiving region 6210 may be as has been previously described. The second assembly part 6064 may be shaped with a concave side in the manner of a lid able to be connected to the first assembly part 6062 around perimeter regions of the respective assembly parts and, when in this connected configuration, forming a cavity between the two parts.

In certain forms, for example as shown in FIGS. 41-44, the first assembly part 6062 and the second assembly part 6064 may be hingedly joined at a hinge 6068, for example along respective sides of the first and second assembly parts. Rotating the parts relative to each other around the hinge 6068 achieves the opening and closing of the cavity. In some forms, for example as illustrated, the hinge 6068 may be a living hinge formed as a thin part of an integral moulding between the two assembly parts. In other forms, the first and second assembly parts may be separately moulded components with hinge 6068 joining them. A hinge connection between the two assembly parts may ensure that the parts are maintained together in use and avoids a user losing one or both parts. In other forms, the first assembly part 6062 and the second assembly part 6064 may be completely separable and formed with portions configured to allow the parts to interconnect with an interlock or friction fit connection.

In certain forms, the first assembly part 6062 and the first assembly component 6002 may be hingedly joined at a hinge 6030, for example along respective sides of the respective part and component. In certain forms, such as illustrated in FIGS. 41-44, this hinged connection may be in addition to the hinged connection between the first assembly part 6062 and the second assembly part 6064. Alternatively, in some forms, the hinge 6030 may be provided instead of hinge 6068. In some forms, the first assembly part 6062 may be hingedly joined to the outer member 6006 of the first assembly component 6002, for example along a side thereof. Rotating the first assembly part 6062 relative to the first assembly component 6002 around hinge 6030 brings the first and second receiving regions together, as previously described. In some forms, for example as illustrated, the hinge 6030 may be a living hinge formed as a thin part of an integral moulding between the first assembly part 6062 and the first assembly component 6002. In other forms, the first assembly part 6062 and the first assembly component 6002 may be separately moulded components with hinge 6030 joining them. A hinge connection between the first assembly part 6062 and the first assembly component 6002 may ensure that the part 6062 and component 6002 are maintained together in use and avoids a user losing one or both. In other forms, the first assembly part 6062 and the first assembly component 6002 may be completely separable and formed with portions configured to allow them to interconnect with an interlock or friction fit connection.

In certain forms, for example the forms shown in FIGS. 41-44, the hinge 6068 may be positioned on one side of the assembly apparatus 6000 and the hinge 6030 may be positioned on the opposite side of the assembly apparatus 6000. That is, hinge 6068 may be provided along one side of the first assembly part 6062 and hinge 6030 may be provided along the opposite side of the first assembly part 6062. This may assist with assembly of the patient interface 3000 in the following manner.

When a user intends to assemble the patient interface 3000, they may begin with the assembly apparatus 6000 in the fully closed configuration, i.e. with the first assembly part 6062 and the second assembly part 6064 closed so that the cavity is formed therebetween, and with the first assembly part 6062 covering the upper surface of the first assembly component 6002. The user may first open the second assembly part 6064 by rotating it around hinge 6068. This opens the cavity (an exemplary configuration of this open configuration is shown in FIG. 43) and allows the user to obtain one of the seal-forming structures 3100 stored in the cavity. The user may then close the second assembly part 6064 by rotating it around hinge 6068 back into engagement with the first assembly part 6062.

The user may then open the first assembly part 6062 relative to the first assembly component 6002 by rotating the first assembly part 6062 relative to the first assembly component 6002 around hinge 6030. In some forms, such as illustrated in FIGS. 41-44, the first and second assembly parts 6062 and 6064 are rotated together relative to the first assembly component 6002. In the illustrated form, this may be conveniently achieved as a continuation of the movement of closing the second assembly part 6064 onto the first assembly part 6062, described above. Once opened, the assembly of the patient interface 3000 may proceed as has been described above, for example in relation to FIGS. 36-40.

In order to adhere the seal-forming structure 3100 to the plenum chamber 3200, a downwards force is applied to the top of the assembly apparatus 6000 when in the closed configuration. With the form of the technology shown in FIGS. 41-44, there is a cavity between the two assembly parts forming the second assembly component 6002. To enable the user to apply a downwards force on central regions of the top surface of the second assembly part 6064 without causing the top surface of the second assembly part 6064 to bow or buckle, a brace 6060 may be provided to span between the inner side of the top surface of the second assembly part 6064 and the top surface of the first assembly part 6062. In some forms, for example as shown in FIGS. 41-44, the brace 6060 is connected to, and projects perpendicularly outwardly from, the top surface of the first assembly part 6062 but in other forms, the brace 6060 may be connected to, and may project perpendicularly outwardly from, the inner side of the top surface of the second assembly part 6064. The brace 6060 may be in the form of an elongate member, for example a post. The brace 6060 may be integrally formed with the respective assembly part or it may be connected thereto. The brace 6060 may be sized with a width smaller than the opening in the central region of the seal-forming structure 3100 so that one or more seal-forming structures 3100 may be stored with the brace 6060 passing through the openings. This may assist with maintaining the seal-forming structure 3100 in the desired position during storage.

### 4.4.7 Feedback on Engagement of Assembly Components

In some forms of the technology, the assembly apparatus 6000 may be configured to provide some form of feedback to the user that the first assembly component 6002 and the second assembly component 6004 have been correctly engaged during the use of the assembly apparatus in order to assemble the patient interface 3000.

In certain forms of the technology, for example as shown in FIGS. 45 and 46, the feedback may be provided in tactile and/or audible form. The assembly apparatus 6000 shown in FIGS. 45 and 46 is similar to the forms shown in FIGS. 36 to 40 and FIGS. 41 to 44. As in those forms, the first assembly component 6002 may comprise an outer member 6006 and an inner member 6008 that are configured to move relative to each other. In moving relative to each other in use of the assembly apparatus 6000, the first receiving region 6110 may be brought into a position adjacent to the second receiving region 6210 to adhere the seal-forming structure 3100 to the plenum chamber 3200, i.e. when the inner and outer members are in the assembly configuration. The assembly apparatus 6000 may comprise two or more components that come into contact through the movement of the outer member 6006 relative to the inner member 6008 between the original configuration and the assembly configuration (these configurations being as described earlier), where the contact is of a nature that generates tactile and/or audible feedback that is able to be detected by the user.

In the example of FIGS. 45 and 46, each of the outer member 6006 and the inner member 6008 comprise a protrusion, the protrusions being positioned and arranged to come into contact with each other through the movement of the outer and inner member relative to each other into the assembly configuration. In other forms, only one of the outer member 6006 and the inner member 6008 may comprise such a protrusion. The protrusion in such forms may be positioned and arranged to contact a part of the other member in the same manner. Contact between the protrusions may generate an audible sound to a user and/or may cause tactile feedback to a user.

As shown in FIGS. 45 and 46, a protrusion 6040 may extend outwardly from a part of the inner member 6008. In the example of FIGS. 45 and 46, the protrusion 6040 extends laterally outwardly from column 6038, although it may extend from other parts of the inner member 6008 in other forms. In the form shown, the protrusion 6040 is a relatively flat tab having a width (i.e. dimension perpendicular to the height of the column 6038) significantly greater than its height (i.e. dimension parallel to the height of the column 6038). During use, when contacted by a part of the inner member 6008 (e.g. its protrusion), the protrusion 6040 may be able to deflect downwardly and/or upwardly. This may generate a sound and/or tactile feedback able to be detected by the user.

As shown in FIG. 45, a protrusion 6042 may extend inwardly from a part of the outer member 6006. In the example of FIG. 45, the protrusion 6042 extends laterally inwardly from an inner surface of the outer wall of the outer member 6006. In the form shown, the protrusion 6042 is in the form of a rounded bulge protruding from the inner surface of the outer wall of the outer member 6006. The roundedness of the bulge may assist in protrusion 6040 moving past the protrusion 6042 in use. The protrusion 6042, or a component it is mounted on, may be able to resiliently deform to prevent abutment of the two protrusions as they move past each other. For example, protrusion 6042 may be mounted on an arm 6044 mounted to the inner surface of the outer wall of the outer member 6006. The arm 6044 may be mounted to the outer wall at one end, i.e. cantilevered, to permit the arm to move in and out. The protrusion 6042 may be mounted at or proximate a distal end of the arm from where it is mounted to the outer wall.

The protrusions 6040 and 6042 may be positioned relative to each other so that they come into contact when the outer member 6006 is moved relative to the inner member 6008 between the original and assembly configurations. For example, the protrusions 6040 and 6042 may be positioned on the same side of the assembly apparatus 6000. In some forms, similar protrusions 6040 and 6042 may be provided on multiple sides of the assembly apparatus 6000 to enable contact between the protrusions irrespective of the orientation in which the outer member 6006 is provided to the inner member 6008. In some forms, multiple protrusions 6040 may be provided, e.g. two protrusions extending outwardly from opposite sides of the column 6038, such as shown in FIG. 45, while only a single protrusion 6042 may be provided. Alternatively, multiple protrusions 6042 may be provided while only a single protrusion 6040 may be provided.

In some forms, the protrusions 6040 and 6042 may be positioned relative to each other so that contact between them, and the consequential audible/tactile feedback, occurs close to the limit of motion of the outer member 6006 relative to the inner member 6008, i.e. at or near the position of the outer and inner members corresponding to the assembly configuration. For example, as is the case for the exemplary form shown in FIGS. 45 and 46, the protrusion 6040 may be located proximate the bottom of the column 6038, near the base 6502 of the inner member 6008, and the protrusion 6042 may be positioned level or close to level with the bottom edge of the outer wall of the outer member 6006. This positioning of the protrusions helps the user know when the first receiving region 6110 is adjacent to the second receiving region 6210 and consequently when the seal-forming structure 3100 and plenum chamber 3200 are brought into contact to adhere them together.

The contact between the two protrusions 6040 and 6042 may result in an increased level of force being necessary to move the protrusions past each other during motion of the outer member 6006 relative to the inner member 6008. Depending on the shape of the protrusions, this may be true for relative movement of the inner and outer members in both directions (i.e. towards the assembly configuration and away from the assembly configuration). An increased level of force being necessary for the inner and outer members to move away from the assembly configuration of the inner and outer members back to the original configuration may help maintain pressure between the first receiving region 6110 and the second receiving region 6210 to help adhere the seal-forming structure 3100 and plenum chamber 3200, even if a user slightly relaxes the force urging the first and second assembly components together.

Exemplary mechanisms for providing audible and/or tactile feedback to the user have been described for a form of the technology similar to the third exemplary form described earlier, but it should be appreciated that similar mechanisms could also be used in other forms, including forms similar to the first and second exemplary forms.

In other forms of the technology, another form of feedback may be provided to the user to illustrate correct engagement of the first and second assembly components, for example visual feedback. In some exemplary forms, the first assembly component 6002 and the second assembly component 6004 may comprise visual indicators that are positioned where they are visible to a user of the device and positioned such that, when the outer member 6006 and the inner member 6008 are brought into the assembly configuration, the visual indicators provide a visual indication of this to the user, for example the visual indicators align with each other. Alternatively, the assembly apparatus 6000 may comprise one or more lights that are configured to illuminate to indicate correct engagement of the first assembly component 6002 and the second assembly component 6004. For example, a sensor may be provided to detect when the first assembly component 6002 and the second assembly component 6004 are moved sufficiently close together, for example when the outer member 6006 and the inner member 6008 are in the assembly configuration, and to cause one or more lights to illuminate as a result.

### 4.4.8 Resiliently Deformable Receiving Region(s)

In certain forms of the technology, the first receiving region 6110 and/or the second receiving region 6210 is/are formed, at least in part, from a resiliently deformable material. The assembly apparatus 6000 may be configured such that, when the first and second assembly components 6002 and 6004 are engaged and urged together, the deformable material is squashed. The effect of this squashing is to spread the deformable material out slightly so that the force exerted on the seal-forming structure 3100 and the adhesive layer 3190 in order to bring them into adhesive contact is more evenly spread across their surface area than it otherwise might be.

In some examples, such as shown in FIG. 41, the second receiving region 6210 may be formed, at least in part, from a resiliently deformable material. For example, the second assembly component 6004 may comprise a pad 6050 that has a surface forming part or all of the second receiving region 6210. The pad 6050 may be formed from a resiliently deformable material, for example a type of rubber such as latex, natural rubber or a synthetic rubber such as silicone rubber.

The pad 6050 may be provided to the second assembly component 6004, for example by being inserted into a hole 6052 in the second assembly component 6004 as shown in FIG. 47. In alternative forms, the pad 6050 may be provided into a depression in the surface of the second assembly component 6004. The pad 6050 may be mounted to the second assembly component 6004 by a friction fit or interlocking arrangement, which may advantageously use the deformability of the pad 6050 to enable the connection of the pad to the second assembly component. In alternative forms, the pad 6050 may be adhered to the second assembly component 6004 using an adhesive.

The pad 6050 may be shaped such that, when provided to the second assembly component 6004, a surface of the pad 6050 is contiguous, or substantially contiguous, with surrounding a surface region of the second assembly component 6004 to form the second region 6200. In some forms, such as shown in FIG. 47, the surface of the pad 6050 facing towards the first assembly component 6002 may lie slightly proud of the surrounding regions of the second assembly component 6004. This may ensure that the pad 6050 comes into contact with the seal-forming structure 3100 first during the assembly process, and may also provide space for the pad 6050 to slightly deform when the first and second assembly components are urged together.

The size of the pad 6050 may be such that it has a breadth in some, and in some forms in all, lateral directions that is similar to, or slightly larger than, the breadth of the seal-forming structure 3100 in the corresponding directions. Consequently, when the second receiving region 6210 is brought into contact with the seal-forming structure 3100 during use, the pad 6050 may be the only part, or substantially the only part, of the second receiving region 6210 to contact the seal-forming structure 3100. This may assist in providing an evenly distributed force across the surface of the seal-forming structure 3100.

As shown in FIG. 47, in some forms the pad 6050 may be connected to, for example may be formed in one-piece with, brace 6060. In the illustrated form, the first assembly part 6062 of the second assembly component 6004 may comprise an insert 6066 where the insert 6066 is a component that comprises the pad 6050 at one end and the brace 6060 at the other end. An intermediate portion of the insert 6066 between the pad 6050 and the brace 6060 is configured to be positioned in the hole 6052 in the second assembly component 6004. The insert 6066 may be an integrally formed one-piece component in some forms, while in other forms it may be an assembly of sub-parts. In use, when a user exerts a force downwardly on the top of the second assembly part 6064, and particularly a middle section of the second assembly part 6064 close to where an end of the brace 6060 abuts against an inner surface of the second assembly part 6064, the force is transmitted downwardly through the brace 6060 to the pad 6050. This enables the user to cause deformation of pad 6050 while pushing down on the seal-forming structure 3100, despite not being able to contact the pad directly during use in the case of certain forms of the technology, such as those illustrated.

There have been described forms of the technology in which the second receiving region 6210 may be formed, at least in part, from a resiliently deformable material. In other forms, the first receiving region 6110 may be formed, at least in part, from a resiliently deformable material instead of, or in addition to, the second receiving region being so formed. For example, a region of the first assembly component 6002 proximate or surrounding the cavity 6124 to accommodate the plenum chamber 3200 may be formed from a resiliently deformable material such as those described earlier. Similarly to as described above, this region may be formed as a separate component that is mounted on the rest of the first assembly component 6002 through a friction fit, interlocking or adhesive connection.

### 4.4.9 Portability

In certain forms of the technology, for example in the case of all the forms of assembly apparatus 6000 shown in the figures, the assembly apparatus 6000 is portable. This may be understood to mean that the assembly apparatus 6000 may have a size and a weight such that it is able to be readily moved and carried by a typical human without mechanical aids. That is, the portability of the assembly apparatuses 6000 distinguishes these forms of the technology from other types of machines that are relatively large and that may be used to assemble patient interfaces in an industrial setting, e.g. factories, which would not be described as portable under this definition.

In certain forms, the mass of the assembly apparatus 6000 may total in the range 50-400 g. For example, in certain forms the mass of the assembly apparatus 6000 may total less than approximately 400 g. In other forms, the mass of the assembly apparatus 6000 may total less than approximately 200 g. the mass of the assembly apparatus 6000 may total less than approximately 100 g.

In certain forms, the dimensions of the assembly apparatus 6000 (e.g. length, depth, height) may be in the range of approximately 40 mm-300 mm. In certain forms, no dimension of the assembly apparatus 6000 may be more than approximately 300 mm. In other forms, no dimension of the assembly apparatus 6000 may be more than approximately 200 mm. In other forms, no dimension of the assembly apparatus 6000 may be more than approximately 150 mm.

In some forms, the assembly apparatus 6000 may be formed from one or more materials that are suitable to achieve a portable apparatus, e.g. an apparatus having dimensions falling in the above-stated ranges and with a mass falling in the above-stated ranges. For example, in certain forms, the first and second assembly components 6002 and 6004 may be formed at least in part from one or more plastics, for example polycarbonate or polypropylene, or from other polymers such as silicone.

In other forms of the technology, the assembly apparatus 6000 may be configured as a non-portable apparatus.

### 4.5 Air Circuit

In one form, the patient interface 3000 may be comprised as part of a patient interface system 5000 which also includes an air circuit 4170. The air circuit 4170 is configured to convey breathable gas to the patient interface 3000 for delivery to the airways of the patient 1000. For example, the air circuit 4170 of the form of the technology shown in FIG. 1 conveys the breathable gas from the RPT device 4000 to the plenum chamber 3200.

A first end of the air circuit 4170 may be connected to the plenum chamber inlet port 3202. A second end of the air circuit 4170, which may be opposite to the first end, may be connected to an RPT device 4000.

In exemplary forms of the technology, the air circuit 4170 is flexible.

In certain forms, the geometric dimensions of the air circuit 4170 may depend on the flow parameters of the breathable gas supplied to the patient from the RPT device 4000. For instance, the diameter of the air circuit 4170 may be relatively small in the case of an RPT device 4000 configured to provide a supply of breathable gas at relatively low pressures (e.g. 2 to 6 cmH₂O), i.e. low pressure therapy. The diameter of the air circuit 4170 may be relatively larger for use with RPT devices 4000 configured to supply breathable gas at higher pressures (e.g. 6 to 20 cmH₂O).

### 4.6 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In certain forms, the RPT device 4000 may be configured to deliver a flow of air to the patient interface 3000 at a positive pressure with respect to ambient. The RPT device 4000 may be configured to deliver air at a therapeutic pressure, for example at least 6 cmH₂O with respect to ambient. Conventional RPT devices 4000 may be used for this purpose.

In other forms, the RPT device 4000 may be configured to deliver a flow or air to the patient interface 3000 at a lower pressure (but still at a positive pressure relative to ambient), for example 2 to 6 cmH₂O with respect to ambient. Respiratory therapy systems incorporating RPT devices 4000 delivering a flow of air at such pressures may be useful for providing low level therapy. For example, such systems may be useful for treating, or ameliorating snoring, or other mild respiratory conditions. Compared to an RPT device that is able to deliver air at higher pressures, for example RPT devices that may be suitable for treating obstructive sleep apnea, such systems may be cheaper to manufacture, use less power and be more compact in size.

FIG. 1 illustrates a respiratory therapy system 2000 incorporating a RPT device 4000 of the type just described. In the form shown in FIG. 1, breathable gas is conveyed to the patient interface 3000 RPT device 4000 which is compact in size and may therefore be portable, i.e. able to be carried by the patient during use, e.g. mounted on the patient's person or clothing. For example, the RPT device 4000 may be strapped around the patient's neck or arm, or carried in a pocket, in use.

Breathable gas from the RPT device 4000 may be conveyed to the patient interface 3000 through an air circuit 4170. The inlet port 3202 of the plenum chamber 3200 may be connected to an end of the air circuit 4170 with the other end of the air circuit 4170 being connected to the RPT device. Since the flow rate and/or pressure of the supply of air may be lower than with a conventional RPT device 4000 (e.g. a CPAP device), the air circuit 4170 may have a reduced diameter compared to conventional air circuits. For example, in certain forms, the air circuit 4170 may have a diameter in the range 5-15 mm, for example 10 mm. A smaller diameter tube provides more impedance to the flow of air than a larger diameter tube but this is acceptable if the flow rate and/or pressure to be delivered is also low. A smaller diameter tube may be desirable as being less bulky and obtrusive, easier to store or package, and cheaper to manufacture.

### 4.7 GLOSSARY

For the purposes of the present technology disclosure, in certain forms of the present technology, one or more of the following definitions may apply. In other forms of the present technology, alternative definitions may apply.

### 4.7.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB (Sleep Disordered Breathing) events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm2 and hectopascal. 1 cmH₂O is equal to 1 g-f/cm2 and is approximately 0.98 hectopascal (1 hectopascal=100 Pa=100 N/m2=1 millibar ˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.7.1.1 Materials

Silicone or Silicone Elastomer: A synthetic rubber. In this specification, a reference to silicone is a reference to liquid silicone rubber (LSR) or a compression molded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.7.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.7.2 Anatomy

### 4.7.2.1 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius):

Lip, upper (labrale superius):

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.7.3 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.8 OTHER REMARKS

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A portable assembly apparatus for assembling a patient interface for use in delivering breathable gas to a patient, wherein the portable assembly apparatus comprises:
a first assembly component comprising a first receiving region configured in use to receive a first portion of the patient interface; and
a second assembly component comprising a second receiving region configured in use to receive a second portion of the patient interface,
wherein the first assembly component is configured to engage with the second assembly component to position the first receiving region adjacent to the second receiving region in a position suitable to adhere the first and second portions together to form an assembled patient interface.

2. The portable assembly apparatus according to claim 1, wherein the first receiving region comprises a substantially continuous surface against which a substantial part of the first portion of the patient interface substantially abuts when the first portion is received by the first receiving region.

3. The portable assembly apparatus according to claim 1, wherein the first receiving region has a shape that substantially corresponds to a natural shape of a non-patient-facing surface of a seal-forming structure of the patient interface to substantially maintain the shape of the seal-forming structure when forming the assembled patient interface.

4. The portable assembly apparatus according to claim 1, wherein the second receiving region comprises a substantially continuous surface against which a substantial part of a patient-facing surface of a seal-forming structure of the patient interface substantially abuts when the first and second portions are adhered together.

5. The portable assembly apparatus according to claim 1, wherein the second receiving region has a shape that is substantially complementary to a shape of the first receiving region such that the first and second receiving regions are capable of being placed in a meshed configuration.

6. The portable assembly apparatus according to claim 1, wherein the second receiving region has a shape that substantially corresponds to a natural shape of a patient-facing surface of a seal-forming structure of the patient interface to substantially maintain the shape of the seal-forming structure when forming the assembled patient interface.

7. The portable assembly apparatus according to claim 1, wherein the first receiving region and/or the second receiving region is/are formed, at least in part, from a resiliently deformable material.

8. The portable assembly apparatus according to claim 1, wherein the first assembly component comprises a first retaining structure which is configured to maintain the first portion in a substantially fixed position with respect to the first assembly component.

9. The portable assembly apparatus according to claim 1, wherein the second assembly component comprises a second retaining structure which is configured to maintain the second portion in a substantially fixed position with respect to the second assembly component.

10. The portable assembly apparatus according to claim 9, wherein the second retaining structure comprises a first slot and a second slot, the first slot being configured to receive a first tab of the second portion or a removable layer connected thereto, and the second slot being configured to receive a second tab of the second portion or a removable layer connected thereto in use.

11. The portable assembly apparatus according to claim 1, wherein the first assembly component and the second assembly component are movable relative to each other such that the portable assembly apparatus has a stable configuration in which the first portion, when received by the first receiving region, and the second portion, when received by the second receiving region, face each other with a gap separating them.

12. The portable assembly apparatus according to claim 11, wherein the first assembly component comprises an outer member and an inner member which is configured to move relative to the outer member to bring the first receiving region into a position adjacent to the second receiving region to adhere the first and second portions together, wherein the inner member comprises the first receiving region.

13. The portable assembly apparatus according to claim 12, further comprising an elastic member which is configured to return the outer member and the inner member to an original configuration when a force causing the inner member to move relative to the outer member is removed.

14. The portable assembly apparatus according to claim 1, wherein the first assembly component is hingedly attached to the second assembly component.

15. The portable assembly apparatus according to claim 1, wherein the first assembly component is detached from the second assembly component.

16. The portable assembly apparatus according to claim 1, wherein the second assembly component comprises a first assembly part comprising the second receiving region and a second assembly part, wherein in a configuration in which the first assembly part and the second assembly part are connected together, the first assembly part and the second assembly part together form a cavity suitable for storing one or more patient interface portions.

17. A method for assembling a patient interface for use in delivering breathable gas to a patient, the method comprising the following steps:
providing a first portion of the patient interface to a first receiving region of a first assembly component of a portable assembly apparatus;
providing a second portion of the patient interface to a second receiving region of a second assembly component of the portable assembly apparatus;
engaging the first assembly component with the second assembly component to position the first receiving region adjacent to the second receiving region in a position suitable to adhere the first and second portions together to form an assembled patient interface.

18. The method according to claim 17, wherein the method comprises providing the first portion to a first retaining structure of the first assembly component to maintain the first portion in a substantially fixed position with respect to the first assembly component.

19. The method according to claim 17, wherein the method comprises providing the second portion to a second retaining structure of the second assembly component to maintain the second portion in a substantially fixed position with respect to the second assembly component.

20. The method according to claim 17, wherein, prior to adhering the first and second portions together, the method comprises moving the first assembly component and the second assembly component into a stable configuration in which the first portion, when received by the first receiving region, and the second portion, when received by the second receiving region, face each other with a gap separating them.
